SEQUENCE LISTING (4 sequences)

SEQ ID NO: 1 - protein, 1079 aa, Homo sapiens
MVCSAAPLLLLATTLPLLGSPVAQASQPVSETGVRPREGLQRRQWGPLIGRDKAWNERIDRPFPACPIPLSSSFGRWPKGQTMWAQTSTLTLTEEELGQSQAGGESGSGQLLDQENGAGESALVSVYVHLDFPDKTWPPELSRTLTLPAASASSSPRPLLTGLRLTTECNVNHKGNFYCACLSGYQWNTSICLHYPPCQSLHNHQPCGCLVFSHPEPGYCQLLPPGSPVTCLPAVPGILNLNSQLQMPGDTLSLTLHLSQEATNLSWFLRHPGSPSPILLQPGTQVSVTSSHGQAALSVSNMSHHWAGEYMSCFEAQGFKWNLYEVVRVPLKATDVARLPYQLSISCATSPGFQLSCCIPSTNLAYTAAWSPGEGSKASSFNESGSQCFVLAVQRCPMADTTYACDLQSLGLAPLRVPISITIIQDGDITCPEDASVLTWNVTKAGHVAQAPCPESKRGIVRRLCGADGVWGPVHSSCTDARLLALFTRTKLLQAGQGSPAEEVPQILAQLPGQAAEASSPSDLLTLLSTMKYVAKVVAEARIQLDRRALKNLLIATDKVLDMDTRSLWTLAQARKPWAGSTLLLAVETLACSLCPQDHPFAFSLPNVLLQSQLFGPTFPADYSISFPTRPPLQAQIPRHSLAPLVRNGTEISITSLVLRKLDHLLPSNYGQGLGDSLYATPGLVLVISIMAGDRAFSQGEVIMDFGNTDGSPHCVFWDHSLFQGRGGWSKEGCQAQVASASPTAQCLCQHLTAFSVLMSPHTVPEEPALALLTQVGLGASILALLVCLGVYWLVWRVVVRNKISYFRHAALLNMVFCLLAADTCFLGAPFLSPGPRSPLCLAAAFLCHFLYLATFFWMLAQALVLAHQLLFVFHQLAKHRVLPLMVLLGYLCPLGLAGVTLGLYLPQGQYLREGECWLDGKGGALYTFVGPVLAIIGVNGLVLAMAMLKLLRPSLSEGPPAEKRQALLGVIKALLILTPIFGLTWGLGLATLLEEVSTVPHYIFTILNTLQGVFILLFGCLMDRKIQEALRKRFCRAQAPSSTISLVSCCLQILSCASKSMSEGIPWPSSEDMGTARS

SEQ ID NO: 2 - DNA, 7873 bp, Homo sapiens
tgggagctgggaatgaggtggaaacccaggacccagaaaagagagggcaggtgcagcgagggagtggtggcggagagagaggactggctctgatcacagtcggacaggtctgtgaccagttctctagcggagaggcctggaaatgaactcatttgtctttgaagcctcatccataaaataggtgttgctggacggatgacatgaagccgtgtatctgaaggcacagtgcctaggggaggacttgctcccttcctgagccctgtctatatgcacctggacaggctgtgggagggggtctgctctgcattcctgggactggccagctaggtgagagaatccagaggggaccggcttgtggcctcgctgcctgtcctctccagctgtcccctctgctcctgtagaatcagcgctggtctccgtctatgtacatctggactttccagataagacctggccccctgaactctccaggacactgactctccctgctgcctcagcttcctcttccccaaggcctcttctcactggcctcagactcacaacaggtaccacttgcgtgggaagggggctgagagtgaatgaacataggctcccgggcctcctgcagccagcttgcctgagactctgtgagcccctctgtatttcctggaggaagggctgcctggttctgtctccgtggcccagctccttcctcacctccctaccagacagacccttccttgcctgccacatccccctatcttctaactttggctgatggcccaagggacagacaacgtgggcccagacctccaccttcacctgttccctggcccccgagacatctgctgcttcgagtcctgactgaggaggcagtcctgatgcatgggcctgactgaggcacctgtagcttggggattggtccagatacccagccctaaagcctctcaggcatcaggcaggtgtctgccctgcccacctagcttcttcagacagcctgcccaccccctcttctcttctctctgtcagagtgtaatgtcaaccacaaggggaatttctattgtgcttgcctctctggctaccagtggaacaccagcatctgcctccattaccctccttgtcaaagcctccacaaccaccagccttgtggctgccttgtcttcagccatcccgaacccgggtactgccagttgctgccacctggtgaggaaggttgggaacttggaaaccaatggccttaagtgaaataaatgttctcagtggttttctcctctctgaacctgtagtttggccagctggtccaagcacagctgctcctctgggtgggagaaaaagccagccatcatagcagatcacaggccctgagcttggaacctgagtagggagactaatgagagaggccccagagacataaggaccaggagagaaagtgctggagtgactgctttttaccttaggaggcaggaagcagctccagtagcccaggatacctgggggagggagaggcatagaccaaaaaggttccctctttggtttccaataacagatagagtcttccaggctggattgcagcagccacattcaggtgcccacccagggacaaaaagaaaaagttaaaaagctagggagggagtgtggaggaatgggctccagagtcaggggagaagccattgctcggctgcatctgagggccataagtccctcctccagggtcccctgtcacctgcctccctgcagtccccgggatcctcaacctgaactcccagctgcagatgcctggtgacacgctgagcctgactctccatctgagccaggaggccaccaacctgagctggttcctgaggcacccagggagccccagtcccatcctcctgcagccagggacacaggtgtctgtgacttccagccacggccaggctgccctcagcgtctccaacatgtcccatcactgggcaggtagccagcctgtcctctccttgcctcctttctccttcctcttacttcccttcatcctcgtcttccttctctgctttccttcacctcttcttcccacgcctccctcccttctccttccttcttttctttccacctctttctcacccttttcatctttccatttacccattctggggaaacaaaggctaagaggtcccttggtgtgaaaaattgcaatgtggaaaattctaaaaatggccagctgttttcactgtggtctgggacttctgagacccttttcagggtttacaaagtcacaactattgtcctaatatgctaagatgtcatttgaccctttcactcccactccctcaggtgtagacagtggccctttccagaggctacagggccatcacgagattgaatgcaaatgcagatgggagaacccagacacgggcaagatttgcaaacatgtaaaacaaagtcacttgtctaattatgttttggaaaatgtagttatttttcataaaaatgtttctgttaacaaaaatactacaattctccacacaaaatatggagaatgtggagaataccgtctcaatgtctgctgagaacagatccatgtttttcaagatgctaaaatggcaggggtggtgcaggaagggcatctgctctagggagagcatgaaattcacgggcatgggccgataaaagagagatctcttctacctcctagaaatccttcttggggacagggaatgtccaccaaaggggccatcctgggaccttgcttgctggggttaagcactgggtggcaggcagaggacaggagcaaggctgtggcttggaaagcagcagagattctgtggtgcagcggggcccagaggagccacatagcgccgcacacacgtttctgcaggtgagtacatgagctgcttcgaggcccagggcttcaagtggaacctgtatgaggtggtgagggtgcccttgaaggcgacagatgtggctcgacttccataccagctgtccatctcctgtgccacctcccctggcttccagctgagctgctgcatccccagcacaaacctggcctacaccgcggcctggagccctggagagggcagcaaaggtatgagaaggggccagcagtcaggggtcagagggaccagggggcagctgtctcttccaggcagctgggtcttcagctcatgagaaacagaggccacagttcaaccagagagtggggtccaaggccaacactgttttctaccccatcagagccatgccacgtctattgccataacataaccacatgtgtataggaaacttttgcaaaatgctgtcatctacacaatctcatttaactctctatggaattagtttgatggtagtctccattttacaaatgaggaaatggtggaaactgagtcctagagcttgttagagaccccacagtcccctccagcaaaatccaagctctcttcctctgtccaagtggagcccacacatcatttggctcttccccactgcttcctctgtttctgaattgctagaaagactgaaacagcatgtcagagcctgctgggttccaggcctgtccctggcccaatgacagttcccttcttcgttttgccttcagcttcctccttcaacgagtcaggctctcagtgctttgtgctggctgttcagcgctgcccgatggctgacaccacgtacgcttgtgacctgcagagcctgggcctggctccactcagggtccccatctccatcaccatcatccagggtacgcagggcctggggcccagtgggctggtcccagctgcttgccttgggagcacgggctctcttgcatggcacgtctctgccctgggcaacaggaccaggcttcggggcccgcatagggttctgcccaaggagaggctcaggtgaggctgtgattgctgagtagcgcctgctcgtcattcttcagatggagacatcacctgccctgaggacgcctcggtgctcacctggaatgtcaccaaggctggccacgtggcacaggccccatgtcctgagagcaagaggggcatagtgaggaggctctgtggggctgacggagtctgggggccggtccacagcagctgcacagatgcgaggctcctggccttgttcactagaaccaaggtgaagcttccaccctgctgcccacgtgccccctccacggcccaccctagcctctctaggacccagcttgcagacccttttccccaaggcccagcccacaggctgttcagcttctctgaagtggagccctagcagagccaggaagtaggagtgagagggcttctgggggtcaacaatctccatgggtctgggatgctcttctcaaaccatcattccaccatgtgtcccacttcatgctgtctcgtctgtctcagctgctgcaggcaggccagggcagtcctgctgaggaggtgccacagatcctggcacagctgccagggcaggcggcagaggcaagttcaccctccgacttactgaccctgctgagcaccatgaaatacgtggccaaggtggtggcagaggccagaatacagcttgaccgcagagccctgaaggtgagatctctgagccacagtgggggccagctgggcagtcgggggctgaagactccccacctgtgggcatttctgtccctctgatgtcaccatgggctgttgggcagcagacctttccagagtccaggggcctgctcctgatccatttctcctctcagacaccactctctgaggctgcagaatggaggcctggcgctgggagcacatgggggttggaggcaggcaagggtgtggagacatgaggcccgaggcgtgtgtgcgcatgcaggcgtgtggctatgatacagacaggaagtttctatggagacgctgaagtatgcttggctttgctgggctcacctaaatcggctctctgtatgggcatccattggtgacccatgagctgcagccaaaagtgtaacaaagggcaatgatattacacaccgtttatgcctgggaatacatggcatgtgtgaatgcacagacatgcgtgtggccgtcgcctccaggacacggtgccctctaccactgctggtcaccattcctagctttgcagacctggaggggccaaagaatgggagaagtcccctcttagaacctgggtggcccctagggatggagggggaagaagggttttcagcagaggggctgggtgcaggtcaggggacatatccttgaagatgccccaggtggttggccaaacagctccctgttcttcccatctagaaagtctcccttcacaggcctgtcttcctctcccttttctctccaaccttgggtcgcacactggactgggaagggaaggtgtggggtctgttgttctcattgcccccggctcagtcctgtgggcgccagcagacggggttcatctttcttttgggtgctgcagaatctcctgattgccacagacaaggtcctagatatggacaccaggtctctgtggaccctggcccaagcccggaagccctgggcaggctcgactctcctgctggctgtggagaccctggcatgcagcctgtgcccacaggaccaccccttcgccttcagcttacccaatgtgctgctgcagagccagctgtttggacccacgtttcctgctgactacagcatctccttccctactcggcccccactgcaggctcagattcccaggcactcactggccccattggtccgtaatggaactgaaataagtattactagcctggtgctgcgaaaactggaccaccttctgccctcaaactatggacaagggctgggggattccctctatgccactcctggcctggtccttgtcatttccatcatggcaggtgaccgggccttcagccagggagaggtcatcatggactttgggaacacagatggttcccctcactgtgtcttctgggatcacagtctcttccagggcagggggggttggtccaaagaagggtgccaggcacaggtggccagtgccagccccactgctcagtgcctctgccagcacctcactgccttctccgtcctcatgtccccacacactgttccggaagaacccgctctggcgctgctgactcaagtgggcttgggagcttccatactggcgctgcttgtgtgcctgggtgtgtactggctggtgtggagagtcgtggtgcggaacaagatctcctatttccgccacgccgccctgctcaacatggtgttctgcttgctggccgcagacacttgcttcctgggcgccccattcctctctccagggccccgaagcccgctctgccttgctgccgccttcctctgtcatttcctctacctggccacctttttctggatgctggcgcaggccctggtgttggcccaccagctgctctttgtctttcaccagctggcaaagcaccgagttctccccctcatggtgctcctgggctacctgtgcccactggggttggcaggtgtcaccctggggctctacctacctcaagggcaatacctgagggagggggaatgctggttggatgggaagggaggggcgttatacaccttcgtggggccagtgctggccatcataggcgtgaatgggctggtactagccatggccatgctgaagttgctgagaccttcgctgtcagagggacccccagcagagaagcgccaagctctgctgggggtgatcaaagccctgctcattcttacacccatctttggcctcacctgggggctgggcctggccactctgttagaggaagtctccacggtccctcattacatcttcaccattctcaacaccctccaggtaggtgataggggggtggctgtgttttttgcttttttagatggtctaagtcactgccgatctcttctctaggaggtaccaaggtggagcagaagaaacataggttcaggaattttggaaggcttaggtgtggatcccagttcctccactgagtagctggataactttggacaaattacataacctctctgagctttggttttcttatctgtaaaataatagctgattttgttggagaaatcaggaaattgtcagtacccaatcctttgctatcccttttataaccataacaataagaaaagcacctgaaatggatcctatgcaccaaatagtggtaacagaaaaattgagatgagaagccttaggatgtgaattacacaggacagaaggagcatgttgattcgggtggatcccttcctccttgaccagcttatccccatgtccctcttctcagggcgtcttcatcctattgtttggttgcctcatggacaggaaggtaagtctgcccacctaaccccctgcctcacttgcagcccgcaggccggggccgtggctggcataagcagagcatttacctctcccgcagatacaagaagctttgcgcaaacgcttctgccgcgcccaagcccccagctccaccatctccctggtgagttgctgccttcagatcctcagctgtgcatccaagagcatgtcagaaggcattccatggccctcctcagaggacatgggcacagccagaagctgagagaagattggggttgttttttagaatgaacagttttccggttccagctccccaccagtggaatgagcagcctggtcagagcagtcaggatcagggtcctgggttcctgattatcacctggactcctgctgactctcttttctctggtttctccatctaaaaatctgcctccagttagcatttgaaggaaaagtgtgggatcagtactcatgggagttactgtagctgagagcaaaatttctaggattcctgcagcacaggcaggagtgcatgtgagaaagtaaaacagatacaacctcttcaagggagagttgacaatactaataactgccctgcaattgggccttcccaccccttcct

SEQ ID NO: 3 - DNA, 3620 bp, Homo sapiens
atcagcaggatggcatcggcaagtcgctcccctcccgggcctcatctgccaaacgatcatctcctcctccgaagttgtatgcatgacaggcgagtggaaacttcactaaaatgaaggcgattgacacaacagaaggaactccatcctttcgggggcttacgaaaataataagtttaaaaaaaataggaagggaattccctcgctccatgatcactgagcgctctcctaaggaaaaggaaatctcccggggggtgccgactacgggcggcgggcttaggatgctcccacgctccccgacccccaatccccaggacccgcaggacctccggaggaacgcccgccagcccgcccggagccacgcggcacaaggtgacacggaccgcgccgcgcgggcccctcagccgcctgggcgaggccgggagcagggagaggggcatccgccggcccgcggtaccttgtacttatcaaagccagccagctgctccgggctcacgtattcgtagccagccatgacgacccgaaaactgagcgcccactcggcagcgactcccggctacaaggctgtgacacacaagcaccacaccggctgggcaaggatggcaaagactgggctgcccgagaagcttcctccttcaacgagtcaggctctcagtgctttgtgctggctgttcagcgctgcccgatggctgacaccacgtacgcttgtgacctgcagagcctgggcctggctccactcagggtccccatctccatcaccatcatccaggatggagacatcacctgccctgaggacgcctcggtgctcacctggaatgtcaccaaggctggccacgtggcacaggccccatgtcctgagagcaagaggggcatagtgaggaggctctgtggggctgacggagtctgggggcccgtccacagcagctgcacagatgcgaggctcctggccttgttcactagaaccaagctgctgcaggcaggccagggcagtcctgctgaggaggtgccacagatcctggcacagctgccagggcaggcggcagaggcaagttcaccctccgacttactgaccctgctgagcaccatgaaatacgtggccaaggtggtggcagaggccagaatacagcttgaccgcagagccctgaagaatctcctgattgccacagacaaggtcctagatatggacaccaggtctctgtggaccctggcccaagcccggaagccctgggcaggctcgactctcctgctggctgtggagaccctggcatgcagcctgtgcccacaggactaccccttcgccttcagcttacccaatgtgctgctgcagagccagctgtttggacccacgtttcctgctgactacagcatctccttccctactcggcccccactgcaggctcagattcccaggcactcactggccccattggtccgtaatggaactgaaataagtattactagcctggtgctgcgaaaactggaccaccttctgccctcaaactatggacaagggctgggggattccctctatgccactcctggcctggtccttgtcatttccatcatggcaggtgaccgggccttcagccagggagaggtcatcatggactttgggaacacagatggttcccctcactgtgtcttctgggatcacagtctcttccagggcagggggggttggtccaaagaagggtgccaggcacaggtggccagtgccagccccactgctcagtgcctctgccagcacctcactgccttctccgtcctcatgtccccacacactgttccggaagaacccgctctggcgctgctgactcaagtgggcttgggagcttccatactggcgctgcttgtgtgcctgggtgtgtactggctggtgtggagagtcgtggtgcggaacaagatctcctatttccgccacgccgccctgctcaacatggtgttctgcttgctggccgcagacacttgcttcctgggcgccccattcctctctccagggccccgaagcccgctctgccttgctgccgccttcctctgtcatttcctctacctggccacctttttctggatgctggcgcaggccctggtgttggcccaccagctgctctttgtctttcaccagctggcaaagcaccgagttctccccctcatggtgctcctgggctacctgtgcccactggggttggcaggtgtcaccctggggctctacctacctcaagggcaatacctgagggagggggaatgctggttggatgggaagggaggggcgttatacaccttcgtggggccagtgctggccatcataggcgtgaatgggctggtactagccatggccatgctgaagttgctgagaccttcgctgtcagagggacccccagcagagaagcgccaagctctgctgggggtgatcaaagccctgctcattcttacacccatctttggcctcacctgggggctgggcctggccactctgttagaggaagtctccacggtccctcattacatcttcaccattctcaacaccctccagggcgtcttcatcctattgtttggttgcctcatggacaggaagatacaagaagctttgcgcaaacgcttctgccgcgcccaagcccccagctccaccatctccctggccacaaatgaaggctgcatcttggaacacagcaaaggaggaagcgacactgccaggaagacagatgcttcagagtgaaccacacacggacccatgttcctgcaagggagttgaggctgtgtgcttgaacccaccagatgagccctggcccaatgctctgaactcttcccgcctcccggagctcagcccttgagaaaggcaggcttatatttcccttagtgacactcatttatcttacagctcaccccttctcatttctaaagtatccagcaagaatagcaggaaaaattagctaaaggcacctaatgaataagcctgcctttgctccagaaataatcgacagatatcaaagtgcggaataattacaagtaaactttctcaaccagtttttaactacaacaatacatgttgtgaatgaatatatttgataaaaatggttttaattgacctattcagcgatttctgattatttctttttcaatagttatgaagaaaggatgacttacttgacaggaacctctgatctttcaaacattggagatgaagggcagaatttggtttgtcttttcaagtttaggaaaaggtgaagttaattggtccctctttctttaacctttaaaaaatcaatataaaatgtaagtttcttaaccatatccatgtatagaggcattgattgatatgagcacgttgtaagaataggttataaaaatttaaagtttaatataaatttatatcaattaataaagtttaatttatatttaaaaatgaatactagaagaaaatctttttgaagacaccaagatatctatctggctgaattaacttatggaattcacaagaggaagatgacaggattctgagaaatttttaaactagatacgtgaaaaaagtctgatgaatcggtctttgttaattatgcaattcatggatattttttataaaatgggacgggggcattttctgttaaaataaaaatggttatgctatc

SEQ ID NO: 4 - DNA, 3240 bp, Homo sapiens
atggtctgttcggctgccccactgctgctcctggccacaactcttcccctgctggggtcaccagttgcccaagcatcccaacctgtaagtgagactggggtgagacccagggaaggtctgcagaggcgacaatggggacccctgattgggagagacaaagcatggaatgaaaggatagacagacccttccctgcctgccccatccccctatcttctagctttggccgatggcccaagggccagacaatgtgggcccagacctccaccctcaccctgacagaggaggagttgggacagagtcaggctggaggggaatctggatctgggcagctcctggaccaagagaatggagcaggggaatcagcgctggtctccgtctatgtacatctggactttccagataagacctggccccctgaactctccaggacactgactctccctgctgcctcagcttcctcttccccaaggcctcttctcactggcctcagactcacaacagagtgtaatgtcaaccacaaggggaatttctattgtgcttgcctctctggctaccagtggaacaccagcatctgcctccattaccctccttgtcaaagcctccacaaccaccagccttgtggctgccttgtcttcagccatcccgaacccgggtactgccagttgctgccacctgggtcccctgtcacctgcctccctgcagtccccgggatcctcaacctgaactcccagctgcagatgcctggtgacacgctgagcctgactctccatctgagccaggaggccaccaacctgagctggttcctgaggcacccagggagccccagtcccatcctcctgcagccagggacacaggtgtctgtgacttccagccacggccaggctgccctcagcgtctccaacatgtcccatcactgggcaggtgagtacatgagctgcttcgaggcccagggcttcaagtggaacctgtatgaggtggtgagggtgcccttgaaggcgacagatgtggctcgacttccataccagctgtccatctcctgtgccacctcccctggcttccagctgagctgctgcatccccagcacaaacctggcctacaccgcggcctggagccctggagagggcagcaaagcttcctccttcaacgagtcaggctctcagtgctttgtgctggctgttcagcgctgcccgatggctgacaccacgtacgcttgtgacctgcagagcctgggcctggctccactcagggtccccatctccatcaccatcatccaggatggagacatcacctgccctgaggacgcctcggtgctcacctggaatgtcaccaaggctggccacgtggcacaggccccatgtcctgagagcaagaggggcatagtgaggaggctctgtggggctgacggagtctgggggccggtccacagcagctgcacagatgcgaggctcctggccttgttcactagaaccaagctgctgcaggcaggccagggcagtcctgctgaggaggtgccacagatcctggcacagctgccagggcaggcggcagaggcaagttcaccctccgacttactgaccctgctgagcaccatgaaatacgtggccaaggtggtggcagaggccagaatacagcttgaccgcagagccctgaagaatctcctgattgccacagacaaggtcctagatatggacaccaggtctctgtggaccctggcccaagcccggaagccctgggcaggctcgactctcctgctggctgtggagaccctggcatgcagcctgtgcccacaggaccaccccttcgccttcagcttacccaatgtgctgctgcagagccagctgtttggacccacgtttcctgctgactacagcatctccttccctactcggcccccactgcaggctcagattcccaggcactcactggccccattggtccgtaatggaactgaaataagtattactagcctggtgctgcgaaaactggaccaccttctgccctcaaactatggacaagggctgggggattccctctatgccactcctggcctggtccttgtcatttccatcatggcaggtgaccgggccttcagccagggagaggtcatcatggactttgggaacacagatggttcccctcactgtgtcttctgggatcacagtctcttccagggcagggggggttggtccaaagaagggtgccaggcacaggtggccagtgccagccccactgctcagtgcctctgccagcacctcactgccttctccgtcctcatgtccccacacactgttccggaagaacccgctctggcgctgctgactcaagtgggcttgggagcttccatactggcgctgcttgtgtgcctgggtgtgtactggctggtgtggagagtcgtggtgcggaacaagatctcctatttccgccacgccgccctgctcaacatggtgttctgcttgctggccgcagacacttgcttcctgggcgccccattcctctctccagggccccgaagcccgctctgccttgctgccgccttcctctgtcatttcctctacctggccacctttttctggatgctggcgcaggccctggtgttggcccaccagctgctctttgtctttcaccagctggcaaagcaccgagttctccccctcatggtgctcctgggctacctgtgcccactggggttggcaggtgtcaccctggggctctacctacctcaagggcaatacctgagggagggggaatgctggttggatgggaagggaggggcgttatacaccttcgtggggccagtgctggccatcataggcgtgaatgggctggtactagccatggccatgctgaagttgctgagaccttcgctgtcagagggacccccagcagagaagcgccaagctctgctgggggtgatcaaagccctgctcattcttacacccatctttggcctcacctgggggctgggcctggccactctgttagaggaagtctccacggtccctcattacatcttcaccattctcaacaccctccagggcgtcttcatcctattgtttggttgcctcatggacaggaagatacaagaagctttgcgcaaacgcttctgccgcgcccaagcccccagctccaccatctccctggtgagttgctgccttcagatcctcagctgtgcatccaagagcatgtcagaaggcattccatggccctcctcagaggacatgggcacagccagaagctga

(19) United States
(12) Patent Application Publication
(10) Pub. No.: US 2019/0265231 A1
(43) Pub. Date: Aug. 29, 2019

(54) GPCR (GPR113) INVOLVED IN FAT, FATTY ACID AND/OR LIPID-ASSOCIATED TASTE AND USE IN ASSAYS FOR IDENTIFYING TASTE MODULATORY

(71) Applicant: SENOMYX, INC., San Diego, CA (US)

(72) Inventors: Stacy Markison ROTH, La Jolla, CA (US); Ginger TOSHIADDI, Oceanside, CA (US); Haining HUANG, San Diego, CA (US); Guy SERVANT, San Diego, CA (US); Mark WILLIAMS, San Diego, CA (US)

(21) Appl. No.: 15/736,907

(22) PCT Filed: Jun. 23, 2016
(86) PCT No.: PCT/US16/39065

§ 371 Date: Dec. 15, 2017

Related U.S. Application Data
(60) Provisional application No. 62/183,312, filed on Jun. 23, 2015.

(51) Int. Cl. G01N 33/50; G01N 33/566; A01K 67/027; C07K 14/705; A61K 49/00

(52) U.S. Cl. CPC G01N 33/5041; G01N 33/566; A01K 67/0276; C07K 14/705; A61K 49/0008; A01K 2267/03; G01N 2500/00; A01K 2207/15; A01K 2217/075; A01K 2227/105; G01N 2333/726

(57) ABSTRACT

This invention relates to a gene encoding a GPR113, wherein GPR113 is a taste receptor polypeptide which detects fat tastants. In one embodiment the invention relates to the use of the GPR113 receptor in screening assays for identifying fat, lipid and fatty acid taste modulators or compounds that mimic fat taste. In another embodiment the invention relates a method for reducing dietary preferences for fat containing foods, comprising administering to a subject a compounds which modulates GPR113. In another embodiment the invention relates to comestibles containing an amount of a compound that specifically binds or modulates GPR113 activity, e.g. a GPR113 enhancer or GPR113 blocker, in an amount sufficient to modulate or mimic fat or lipid taste or to affect fat or lipid metabolism.

### RELATED APPLICATIONS

This application is a U.S. National Phase application of International Appl. No. PCT/US2016/039065, filed Jun. 23, 2016, which claims priority to U.S. Provisional Appl. No. 62/183,312, filed Jun. 23, 2015, each of which is incorporated herein by reference.

### SEQUENCE LISTING

The sequence listing in the file named “43268o4014.txt” having a size of 29,353 that was created Dec. 13, 2017, is hereby incorporated by reference in its entirety.

### FIELD OF THE INVENTION

This invention relates to a gene encoding a GPCR that is involved in fat, lipid and fatty acid associated taste and potentially physiological functions involving lipid, fat and fatty acid absorption, excretion and metabolism, and dietary fat consumption and body weight control. This gene was initially identified as encoding a taste specific G protein coupled receptor polypeptide based on different criteria including its level of expression and enrichment in the top fraction of taste bud (TB) cells, where all other taste receptor gene mRNAs are enriched and the fact that this genes is co-expressed in a subset of taste cells which express T1R3, which receptor comprises part of heteromeric taste receptors which detect sweet and umami tastants. As disclosed infra behavioral assays in rodents wherein the expression of this gene is knocked out and other assays have established that this gene encodes a GPCR which detects the taste of different fats, lipids and fatty acids.

Based thereon, this invention relates to assays using this gene and the corresponding receptor polypeptide for identifying compounds that enhance or block fat, lipid or fatty acid taste and/or which modulate fat, lipid or fatty acid absorption, excretion and metabolism and/or which modulate dietary fat consumption preference. These compounds will have application as flavor additives in comestibles and other compositions for human consumption and potentially may have application as therapeutics in subjects in need thereof, e.g., individuals with conditions resulting in aberrant lipid or fat or fatty acid metabolism or individuals with food related disorders such as obesity, type 2 diabetes, metabolic syndrome, and fatty liver disease. Also probes can be constructed based on the GPR113 sequence to identify endogenous cells, preferably human, non-human primate and other mammalian cells that are involved in fat, lipid and fatty acid associated taste and potentially physiological functions involving lipid, fat and fatty acid absorption, excretion and metabolism, and dietary fat consumption and body weight control.

### BACKGROUND OF THE INVENTION

During the past decade the understanding of mammalian taste and especially human taste has become much more understood. In particular, genomic based research methods have revealed the identity of specific genes and gene families which are involved in different taste modalities including bitter, sweet, umami and sour. This research has revealed the identity of specific GPCRs which are expressed in human and other mammalian taste bud cells and are involved in taste transduction.

For example research by the present Assignee Senomyx as well as the University of California has revealed the existence of a GPCR family generally referred to in the literature as the T1R family that includes three genes, T1R1, T1R2 and T1R3. These genes encode GPCR taste receptor polypeptides which when expressed as monomers or as heteromers (i.e., T1R2/T1R3 or T1R1/T1R3) specifically respond to sweet or umami taste stimuli. Also, the subject Assignee and others have identified another family of GPCRs referred to in the literature as T2Rs which family of taste receptors is involved in bitter taste transduction. This gene family in humans includes 25 members which respond to different bitter taste ligands. Further, research by scientists at Duke University and the University of California has revealed the identity of two ion channels, PDK2L1 and PKD1L3 which reportedly are involved in sour taste transduction.

Less is known about how humans or other mammals perceive fat taste. The detection of fat in the mouth has traditionally been considered to rely on texture, viscosity and smell. However, some fat replacers which mimic these qualities do not adequately mimic the mouth sensation and pleasure of fat. Partly for this reason, it was theorized by the present Applicant and others that there may be a fat taste receptor. However, its identity and even the type of proteins it might be, e.g., ion channel, GPCR or another type of protein was unknown.

Related to the foregoing fMRI studies have shown that vegetable oil stimulates the taste areas of the human cortex and nerve recordings in rats have shown that free fatty acid (FFA) application to the tongue stimulates the lingual branch of the glossopharyngeal nerve. This result suggests that the fat sensation has an extra-trigeminal component. It has also been observed that isolated rat taste cells respond to medium and long chain FFAs by inhibiting a delayed rectifying potassium channel. Thus, several lines of evidence suggest that medium and long chain FFA's are capable of eliciting fat taste.

Systems for screening compounds that elicit a fat taste but which are not themselves fat are needed in the food industry. Such systems could be used to identify compounds that can replace fat in foods thereby providing healthier foods having fewer calories but that retain desirable flavor characteristics.

Damak et al and others have reported e.g., in US20080299270 and in J. Neurosci. 30(25):8376-82 (2010) that GPR40 and GPR120 are purportedly fat taste receptors and allegedly may be used in screens to identify compounds that mimic or modulate fat taste. Also, Laugerette et al., J Clin. Invest. 115(11):3177-84 (November 2005) allege that CD36 is involved in sensory detection of dietary lipids, spontaneous fat preference and digestive secretions.

Further, Mattes. doi:10.1016/j.physbeh.2011.02.016 (2011) review mechanisms of detection of dietary fats in the oral cavity and intestines and fat signaling processes via tactile and retronasal olfactory cues and suggest that these processes are involved in fat absorption, energy intake and appetite regulation. In addition, Stewart et al, British Journal of Nutrition 104(1):145-152(2010) have suggested that genetic factors may affect dietary fat consumption and may affect body weight control. Also, Mattes in Am J. Gastrointest. Liver Phys. 296:G365-371 (2009) teaches that oral stimulation, especially oral fat exposure elevates serum triglycerides in humans.

### BRIEF DESCRIPTION AND OBJECTS OF THE INVENTION

This invention in one embodiment relates to the discovery that a GPCR generally referred to in the scientific literature as GPR113 or G protein coupled receptor 113 encodes a taste receptor polypeptide which detects fat tastants.

GPR113 was first discovered in 2002 (Fredriksson et al, FEBS Lett., 2002) and later found to be expressed in mouse taste buds (LopezJimenez et al, Genomics, 2005). GPR113 was previously reported to be lingually expressed and to be expressed by circumvallate (CV) taste buds of humans, primates, and rodents. However, the function of this gene in taste was not previously known. Moreover, it was not even clear that this gene elicited any role in taste perception.

The function of GPR113 was discovered in part by use of knockout mouse models. Particularly, the inventors generated a knockout mouse model of GPR113 (GPR113 KO) and using this animal model it was shown that GPR113 KO mice have impaired responsiveness to fat stimuli using a variety of behavioral paradigms. These findings suggested that GPR113 is necessary for normal responsiveness to fats such as soybean oil and corn oil as well as fatty acids such as linoleic acid and oleic acid.

In addition, the inventors conducted further animal studies in order to confirm this prediction. As described infra the inventors compared licking profiles from wild-type mice with glossopharyngeal nerve transection (GLX) with GPR113 knockout (GPR113 KO) and show that GLX mice relative to their sham transected counterparts have decreased licking responses to soybean oil but not sucrose. These findings further corroborate that GPR113 encodes a receptor polypeptide responsive to fats, fatty acids, and lipids.

Based thereon, in one embodiment the invention relates to the use of the GPR113 receptor in screening assays for identifying fat, lipid and fatty acid taste modulators or compounds that mimic fat taste.

In addition, as this receptor mediates sensory signals with different fats, lipids and fatty acids, this receptor when expressed on gastrointestinal cells or other endogenous cells such as liver cells, gall bladder cells, pituitary cells, and neural cells, and that GPR113 may play a role in fat metabolism. Accordingly in another embodiment the invention relates to the use of GPR113 in assays to identify compounds that modulate fat, fatty acid or lipid absorption, excretion or metabolism, and dietary fat consumption and body weight control.

Also in another embodiment the invention relates to the administration to subjects of compounds which modulate GPR113, i.e., as food additives or in medicaments in order to affect (typically reduce) dietary preferences for fat containing foods compounds or in order to affect (typically reduce) dietary preferences for fat containing foods.

In another embodiment the invention relates to comestibles containing an amount of a compound that specifically binds or modulates GPR113 activity, e.g. a GPR113 enhancer or GPR113 blocker, in an amount sufficient to modulate or mimic fat or lipid taste or to affect fat or lipid metabolism.

In another embodiment the invention relates to assays that identify compounds that modulate the function of GPR113 and the use of the identified compounds to modulate fat taste perception in humans and other animals.

In another embodiment the invention relates to the discovery that GPR113-specific probes including GPR113-specific nucleic acids, polypeptides and antibodies can be used to identify, purify or isolate fat taste bud cells, fat taste bud committed stem cells or immature taste cells that are differentiating into mature fat taste bud cells. In addition these probes may be used to detect cells that endogenously express GPR113 that may be used in assays to screen for compounds that modulate fat, lipid and fatty acid associated taste and potentially physiological functions involving lipid, fat and fatty acid absorption, excretion and metabolism, and dietary fat consumption and body weight control.

In another embodiment the invention provides the discovery that GPR113 and compounds that enhance or inhibit this gene product can selectively modulate fat or lipid taste cell function and responses to fat and lipid tastants and may regulate dietary fat consumption and thereby be useful in controlling body weight.

### BRIEF DESCRIPTION OF THE FIGURES

FIG. 1 contains an example of laser capture microdissection (LCM) on human taste buds. The figure contains three panels. In panel A is shown a methyl blue stained section of human circumvallate taste buds. In panel B is contained cell section A following the excision of human taste buds. Panel C shows the captured human taste buds.

FIG. 2 contains a double label in situ hybridization experiment. This hybridization experiment used primate circumvallate papilla and revealed that the taste cell specific gene GPR113 (purple color; left image) colocalizes with a subset of TRPM5 cells (red; middle image). It can be seen from the figure that that only a fraction of cells expressing TRPM5, a marker of sweet, umami, and bitter taste cells, also express GPR113 (merged image on the right), but that all GPR113 cells express TRPM5. Two taste buds are shown.

FIG. 3 shows that GPR113 is not expressed in T1R1 umami cells. Double label in situ hybridization of primate circumvallate papilla showing that GPR113 (purple color; left image) does not colocalize with T1R1 (red; middle image). Note that GPR113 and T1R1, a marker of umami cells, are in different taste cells (merged image on the right).

FIG. 4 shows that GPR113 is not expressed in T1R2 sweet cells. Double label in situ hybridization of primate circumvallate papilla showing that GPR113 (purple color; left image) does not colocalize with T1R2 (red; middle image). Note that GPR113 and T1R2, a marker of sweet cells, are in different taste cells (merged image on the right).

FIG. 5 shows that GPR113 is expressed in a subset of T1R3 cells. Double label in situ hybridization of primate circumvallate papilla showing that GPR113 (purple color; left image) does colocalize with a subset of T1R3 cells (red; middle image). Note that GPR113 is always expressed in cells with T1R3, but that there are T1R3 cells that do not express GPR113 (merged image on the tight). These T1R3 cells that do not express GPR113 likely coexpress either T1R1 or T1R2. The T1R3 only cells are a new population of taste cells that coexpress GPR113. The GPR113 genes and the T1R3 gene may multimerize in these cells such as is the case with T1R3 and other taste receptor polypeptides (T1R2 and T1R3).

FIG. 6 shows that GPR113 is not expressed in T2R bitter cells. Double label in situ hybridization of primate circumvallate papilla showing that GPR113 (purple color; left image) does not colocalize with T2R (red; middle image). Note that GPR113 and T2R, a marker of bitter cells, are in different taste cells (merged image on the right).

FIG. 7 shows ISH expression of GPR113 in wild-type (WT) and GPR113 knockout (KO) mice.

FIG. 8 shows mean (±SE) percent preference to a range of soybean oil concentrations measured over 2, 24-hour periods in two-bottle testing in wild-type (WT; closed circles) and GPR113 knockout (KO; open circles) mice.

FIG. 9 shows mean (±SE) percent preference to a range of polycose concentrations measured over 2, 24-hour periods in two-bottle testing in wild-type (WT; closed circles) and GPR113 knockout (KO; open circles) mice.

FIG. 10 contains mean (±SE) number of licks taken to a range of soybean oil concentrations and the vehicle emplex measured during 5-second trials in wild-type (WT; closed circles) and GPR113 knockout (KO; open circles) mice.

FIG. 11 contains mean (±SE) number of licks taken to a range of mineral oil concentrations and the vehicle emplex measured during 5-second trials in wild-type (WT; closed circles) and GPR113 knockout (KO; open circles) mice.

FIG. 12 shows that the licking profiles from mice with glossopharyngeal nerve transection (GLX) mimic that of GPR113 knockout (GPR113 KO). The figure shows that GLX mice relative to their sham transected counterparts have decreased licking responses to soybean oil but not sucrose.

FIG. 13 contains the results of experiments wherein GPR113 was transiently co-expressed with various G proteins and basal levels of IP1 in cells were measured with an HTRF-based kit from Cisbio.

FIG. 14 contains the results of experiments wherein GPR113 or control receptors were co-expressed with varying amounts of Gq and IP1 levels measured with the Cisbio kit. GPR113 isoforms I and III consistently generated higher IP1 levels than the negative controls, T1R3 or a GPR113 construct containing a frame-shift mutation (GPR113-null).

FIG. 15 contains the results of experiments wherein constitutive GPR113 activity was measured in an ELISA-based cAMP assay (Perkin Elmer) in which GPR113 or a histamine receptor, H1R, is co-expressed with a G protein chimera, Gsq5. This chimera consists of the Gs subunit with a substitution of the last 5 amino acids from Gq.

FIG. 16 contains the results of experiments wherein GPR113 or control receptors were co-expressed with varying amounts of Gq and IP1 levels measured with the Cisbio kit.

FIG. 17 contains the results of experiments wherein GPR113 or control receptors were co-expressed with varying amounts of the Gsq5 chimeric G-protein and cAMP levels measured with the ELISA-based cAMP kit.

FIG. 18 contains the results of experiments wherein GPR113 was co-expressed with varying amounts of Gs or the Gsq5 chimeric G-protein and cAMP levels measured with the ELISA-based cAMP kit.

FIG. 19 contains the results of experiments wherein GPR113 or a control null receptor were co-expressed with Gq and the effect of two novel agonists (compounds A and B) and one novel antagonist (compound C) on the IP1 levels were evaluated with the Cisbio kit.

FIG. 20 contains the results of experiments wherein GPR113 or a control null receptor were co-expressed with Gsq5 and the effect of two novel agonists (compounds A and B) and one novel antagonist (compound C) on the cAMP levels were evaluated with the ELISA-based cAMP kit.

### DETAILED DESCRIPTION OF THE INVENTION

The present application is based on the discovery that the GPR113 gene encodes a taste specific GPCR polypeptide which detects fat tastants and which is involved in fat taste regulation. Based on this discovery GPR113 polypeptides and cells which express same may be utilized in assays for identifying compounds that mimic fat taste or which regulate fat taste perception or fat absorption and metabolism. Such compounds can be incorporated into foods as fat replacers or to modulate fat taste perception or in medicaments or comestibles to modulate fat metabolism and regulate dietary fat consumption and body weight control.

As reported in the examples, GPR113 gene knockout mice, relative to the wild-type mice, exhibit reduced responsiveness to different fats and oils including different soybean oil and corn oil compositions as well as to the fatty acids linoleic acid and oleic acid. By contrast, the knockout and wild-type mice showed no difference in taste responsiveness to other (non-fat) tastants (sweet, bitter, salt, sour) such as polycose, sucrose, NaCl, KC, citric acid and quinine. In addition there was no difference in responsiveness to a tasteless oil, mineral oil, confirming that the responsiveness of GPR113 to different fats and its modulatory effect on fat intake is taste specific, i.e., it is not a function of viscosity or “mouth-feel”.

Based thereon this taste receptor and cells which express GPR113, both recombinant and endogenous taste cells, may be used in screens, e.g., high-throughput screens in order to identify enhancers and blockers of fat taste as well as compounds that mimic fat taste. Also, the effects of the identified compounds on fat taste may be verified in human or animal taste tests, i.e., to determine if the identified compounds augment or repress fat taste perception or elicit a fatty taste.

Therefore the present invention includes the use of cell-based assays to identify fat taste modulators (e.g., agonists, antagonists, enhancers, blockers) using endogenous or recombinant cells which express GPR113 polypeptides. These cells may also express T1R3 and/or TRPM5. These compounds have potential application in modulating human taste perception to different fats, oils, lipids and fatty acids and may affect other fat related physiological functions including fat absorption and metabolism, or the hedonic response to fats as it relates to dietary control and preference

Compounds identified in screening assays, e.g., electrophysiological assays, FFRET assays and their biologically acceptable derivatives are to be tested in human taste tests using human volunteers to confirm their effect on fat taste perception. In addition compounds identified as potential therapeutics for modulating fat absorption or metabolism will be evaluated in appropriate in vitro and in vivo models depending on the nature of the intended application. For example compounds identified as potential therapeutics for treating diabetes or obesity may be evaluated in well-known diabetic or obesity animal models such the db/db mouse, Zucker fatty rat, ZDF rat, and diet-induced obese rodent models. Similarly, compounds identified as potential therapeutics potentially may be used to treat Irritable Bowel Syndrome (IBS) or Crohn's disease, gall bladder related diseases or syndromes, or liver diseases and other diseases involving aberrant fat metabolism. The efficacy of these compounds as putative therapeutics may be tested in appropriate in vitro or animal models for the particular disease or condition.

As discussed further infra, the cell-based assays used to identify fat taste modulatory or therapeutic compounds will preferably comprise high throughput screening platforms to identify compounds that modulate (e.g., agonize, antagonize, block or enhance) the activity of GPR113 using cells that express the GPR113 gene disclosed herein optionally with other taste specific genes or combinations thereof. Additionally, these sequences may be modified to introduce silent mutations or mutations having a functional effect such as defined mutations that affection (sodium) influx. The assays may comprise fluorometric or electrophysiological assays effected in amphibian oocytes or assays using mammalian cells that express the subject GPCR. Also, compounds that modulate GPR113 putatively involved in taste may be detected by ion flux assays, e.g., radiolabeled-ion flux assays or atomic absorption spectroscopic coupled ion flux assays or label-free optical biosensor assays. As disclosed supra, these compounds have potential application in modulating human fat taste perception or for modulating other biological processes involving fat absorption and metabolism and diseases such as autoimmune disorders involving aberrant fat metabolism or elimination.

The subject cell-based assays use wild-type or mutant nucleic acid sequences which are expressed in desired cells, such as oocytes, insect or human cells such as CHO, COS, BHK, STO or other human or mammalian cells conventionally used in screens for GPCR modulatory compounds. These cells may further be engineered to express other sequences, e.g., other taste GPCRs, e.g., T1Rs or T2Rs such as T1R3 as well as appropriate G proteins and/or taste specific ion channels such as TRPM5 or TRPM8. The oocyte system is advantageous as it allows for direct injection of multiple mRNA species, provides for high protein expression and can accommodate the deleterious effects inherent in the overexpression of ion channels. The drawbacks however are that electrophysiological screening using amphibian oocytes is not as amenable to high throughput screening of large numbers of compounds and is not a mammalian system. As noted, the present invention embraces assays using mammalian cells, preferably high throughput assays.

In an exemplary embodiment high throughput screening assays are effected using mammalian cells transfected or seeded into wells or culture plates wherein functional expression in the presence of test compounds is allowed to proceed and activity is detected using calcium, membrane-potential fluorescent or ion (sodium) fluorescent dyes. However, as described infra this fluorescent assay is exemplary of assay methods for identifying compounds that modulate GPR113 function and the invention embraces non-fluorescent assay methods.

The invention specifically provides methods of screening for modulators, e.g., agonists, antagonists, activators, inhibitors, blockers, stimulators, enhancers, etc., of human fat taste and taste sensation (intensity) and potential therapeutics that target other taste cell functions or phenotypes using the nucleic acids and proteins, sequences provided herein. Such modulators can affect fat taste and taste cell related functions and phenotypes, e.g., by modulating transcription, translation, mRNA or protein stability; by altering the interaction of the polypeptide with the plasma membrane, or other molecules; or by affecting GPR113 protein activity.

Compounds are screened, e.g., using high throughput screening (HTS), to identify those compounds that can bind to and/or modulate the activity of the subject fat taste receptor or fragment thereof. In the present invention, the subject GPR113 proteins alone or in association with T1R3 and/or TRPM5 are recombinantly or endogenously expressed in cells, e.g., human cells, other mammalian cells, or frog oocytes and the modulation of activity is assayed by using any measure of GPCR function, such as binding assays, conformational assays, calcium based assays, measurement of the membrane potential, measures of changes in intracellular sodium or lithium levels, or optical biosensor changes. More specifically, the assays may use human, non-human primate or other mammalian cells which endogenously express one or more of GPR113, TRPM5 and T1R3. These cells may further endogenously express a G protein or a nucleic acid may be introduced therein encoding a G protein such as Ga15, Ga16, transducin or gustducin or a chimera of any of the foregoing such as Ga15 or Ga16/gust44 or G_{α}15 or Ga16/transducin44 wherein the C-terminal 44 amino acids of Ga15 or Ga16 are substituted for the corresponding 44 amino acids of gustducin or transducin.

Methods of assaying ion, e.g., cation, channel function include, for example, patch clamp techniques, two electrode voltage clamping, measurement of whole cell currents, and fluorescent imaging techniques that use ion⁻ sensitive fluorescent dyes and ion flux assays, e.g., radiolabeled-ion flux assays or ion flux assays. Other assays are exemplified infra.

An enhancer or activator of GPR113 or a compound that specifically binds GPR113 identified according to the current application can be used for a number of different purposes. For example, it can be included as a flavoring agent to modulate enhance) the taste of foods, beverages, soups, medicines, and other products containing a fat, oil, lipid, or fatty acid which is for human consumption. Additionally, the invention provides kits for carrying out the herein-disclosed assays. Compounds identified using these assays that specifically bind or modulate the activity of GPR113 alone or when GPR113 is expressed in association with T1R3 and/or TRPM5, e.g., enhancers or activators, may also be used to modulate fat metabolism and diet control as discussed previously.

Also as noted previously the present invention particularly provides the use of the subject taste specific gene as a marker which can be used to enrich, identify or isolate specific taste cell subsets or to enrich, identify or isolate fat taste bud committed stem cells and/or cells that modulate fat metabolism and diet control.

Prior to discussing the present invention in more detail the following definitions are provided. Otherwise all terms are to be accorded their ordinary meaning as they would be understood by one skilled in the relevant field of endeavor.

### Definitions

“Putative taste receptor” refers to a gene expressed in taste cells that is not expressed in lingual epithelial cells or is expressed substantially less in lingual epithelial cells. This includes chemosensory or taste cells, particularly those of human or macaque and other animals, especially other mammals.

“Taste Cell” refers to a cell that when mature expresses at least one receptor, transporter, or ion channel that directly or indirectly regulates or modulates a specific taste modality such as sweet, sour, umami, salty, bitter, fatty, metallic, CO₂ or other taste perception or general taste perception such as taste intensity or the duration of a taste response. Taste cells can express mRNA and/or a protein for the gene C6orf15 (chromosome reading frame 15)—also known as STG. This gene has been described as a taste-specific gene (M. Neira et al. Mammalian Genome 12: 60-66, 2001). Herein these cells specifically include any mammalian cell, preferably human or non-human primate cells, that endogenously or recombinantly express GPR113 and which may further express T1R3 and/or TRPM5. These GPR113 expressing cells involved in fat taste, metabolism and fat datary control cells may be located on the tongue as in taste buds or may be comprised in other organs such a in the gastrointestinal system (e.g., the stomach, intestines, colon, liver, gall bladder), on neural cells and other endogenous cells.

“Chemosensory cells” are cells that are involved in sensing of chemical stimulants such as tastants and other chemical sensory stimuli such as odorants. Chemosensory cells herein include in particular taste cells and cells comprised in the digestive or urinary tract or other organs that when mature express one or more taste receptors such as GPR113. For example, gastrointestinal chemosensory cells are known which express T1Rs or T2Rs and which cells are likely involved in food sensing, metabolism, digestion, glucose metabolism, food absorption, gastric motility, et al. As mentioned herein GPR113 may be expressed on different endogenous cells such as cells located on the tongue as in taste buds or may be comprised in other organs including by way of example organs in the gastrointestinal system (e.g., the stomach, intestines, colon, liver, gall bladder), on neural cells and other endogenous cells. In addition, cells found in the urinary tract likely express salty taste receptors and are involved in sodium transport, excretion and functions associated therewith such as blood pressure and fluid retention. Further, in the digestive system chemosensory cells that express taste receptors may also express chromogranin A, which is a marker of secretory granules. (C. Sternini, “Taste Receptors in the Gastrointestinal Tract, IV, Functional Implications of Bitter Taste Receptors in Gastrointestinal Chemosensing” American Journal of Physiology, Gastrointestinal and Liver Physiology., 292:G457-G461, 2007).

“Taste-cell associated gene” herein refers to a gene expressed by a taste cell that is not expressed by lingual epithelial cells that is involved in a taste or non-taste related taste cell function or phenotype. Taste cells include cells in the oral cavity that express taste receptors such as the tongue and palate, and taste cells in other areas of the body that express taste receptors such as the digestive system and urinary tract. Such genes include those contained herein. These genes include genes involved in taste and non-taste related functions such a taste cell turnover, diseases affecting the digestive system or oral cavity, immunoregulation of the oral cavity and/or digestive system, digestive and metabolic functions involving taste cells such a diabetes, obesity, blood pressure, fluid retention et al. In referring to the particular taste specific gene identified herein these genes include the nucleic acid sequences corresponding to the genes as well as orthologs thereof and chimeras and variants including allelic variants thereof. In particular such variants include sequences encoding polypeptides that are at least 80% identical, more preferably at least 90% or 95% identical to the polypeptides encoded by the gene or to orthologs thereof, especially human and non-human primate orthologs. In addition, the genes include nucleic acid sequences that hybridize under stringent hybridization conditions to a nucleic acid sequence corresponding to the identified GPCR taste bud specific gene sequence.

The term “endogenous GPR113 expressing cell” herein refers to any cell that endogenously, i.e., natively express a chromosomal DNA that encodes a GPR113 receptor polypeptide.

The term “authentic” or “wild-type” or “native” nucleic acid sequences refer to the wild-type nucleic acid sequence encoding the taste specific gene provided herein as well as splice variants and other nucleic acid sequences generally known in the art. Herein this refers to GPR113 wild-type nucleic acid sequences.

The term “authentic” or “wild-type” or “native” polypeptides refer to the polypeptide encoded by the genes and nucleic acid sequence contained herein. Herein this refers to GPR113 wild-type polypeptide sequences.

The term “modified or enhanced receptor nuclear acid sequence” or “optimized nucleic acid sequence” refers to a nucleic acid sequence that contains one or more mutations, particularly those that affect (inhibit or enhance) gene activity in recombinant host cells, and most especially oocytes or human cells such as CHO, COS, BHK, frog oocytes or other mammalian cells. The invention embraces the use of other mutated gene sequences, i.e., splice variants, those containing deletions or additions, chimeras of the subject sequences and the like. Further, the invention may use sequences which may be modified to introduce host cell preferred codons, particularly amphibian or human host cell preferred codons.

The term receptor or fragment thereof, or a nucleic acid encoding a particular taste receptor or ion channel or transporter or a fragment thereof according to the invention refers to nucleic acids and polypeptide polymorphic variants, alleles, mutants, and interspecies homologs that: (1) have an amino acid sequence that has greater than about 60% amino acid sequence identity, 65%, 70%, 75%, 80%, 85%, 90%, preferably 91%, 92%, 93%, 94%, 95%, 96%, 97%, 98% or 99% or greater amino acid sequence identity, preferably over a region of at least about 25, 50, 100, 200, 500, 1000, or more amino acids, to an amino acid sequence encoded by the wild-type nucleic acid or amino acid sequence of the taste protein, e.g., proteins encoded by the gene nucleic acid sequences contained herein as well as fragments thereof, and conservatively modified variants thereof; (2) polypeptides encoded by nucleic acid sequences which specifically hybridize under stringent hybridization conditions to an anti-sense strand corresponding to a nucleic acid sequence encoding a gene encoded by one of said genes, and conservatively modified variants thereof; (3) have a nucleic acid sequence that has greater than about 60% sequence identity, 65%, 70%, 75%, 80%, 85%, 90%, preferably 91%, 92%, 93%, 94%, 95%, 96%, 97%, 98% or 99%, or higher nucleotide sequence identity, preferably over a region of at least about 25, 50, 100, 200, 500, 1000, or more nucleotides, to a nucleic acid, e.g., those disclosed herein.

By “determining the functional effect” or “determining the effect on the cell” is meant assaying the effect of a compound that directly or indirectly affects the activity of the subject GPCR polypeptide, i.e., GPR113. For example such compound may specifically bind or activate GPR113 or may enhance, promote or block the binding or activation of GPR113 by a specific ligand such as a fat, oil, lipid or fatty acid. These compounds may be used to enhance, block or mimic fat taste. Alternatively such compound may increase or decrease a parameter that is indirectly or directly under the influence of the subject GPCR polypeptide, e.g., functional, physical, phenotypic, and chemical effects. Such functional effects include, but are not limited to, changes in ion flux, second messengers, membrane potential, current amplitude, and voltage gating, as well as other biological effects such as changes in gene expression of any marker genes, and the like. The second messengers can include, e.g., cyclic AMP, inositol phosphates, diacyl glycerol, or calcium. The ion flux can include any ion that passes through the channel, e.g., sodium, lithium, potassium, or calcium and analogs thereof such as radioisotopes. Such functional effects can be measured by any means known to those skilled in the art, e.g., patch clamping, using voltage-sensitive dyes, or by measuring changes in parameters such as spectroscopic characteristics (e.g., fluorescence, absorbance, refractive index), hydrodynamic (e.g., shape), chromatographic, or solubility properties.

“Inhibitors”, “Agonists”, “Antagonists”, “Activators,” Blockers”, and “Modulators” of the subject fat taste receptor gene and polypeptide sequences are used to refer to compounds that specifically bind or affect the activity of GPR113 in an in vitro or in vivo assay or which modulate (enhance or block) the binding or activation of GPR113 by another compound such as a fat, oil, lipid or fatty acid. This includes by way of example activating, inhibiting, or modulating molecules identified using in vitro and in vivo assays including the subject GPR113 encoding polynucleotide and polypeptide sequences. Inhibitors or blockers or antagonist compounds are compounds that, e.g., bind to, partially or totally block activity, decrease, prevent, delay activation, inactivate, desensitize, or down regulate the activity or expression of these taste specific proteins, e.g., antagonists. “Activators” are compounds that increase, open, activate, facilitate, enhance activation, sensitize, agonize, or up regulate protein activity. Inhibitors, activators, or modulators also include genetically modified versions of the subject taste cell specific proteins, e.g., versions with altered activity, as well as naturally occurring and synthetic ligands, antagonists, agonists, peptides, cyclic peptides, nucleic acids, antibodies, antisense molecules, siRNA, miRNA, ribozymes, small organic molecules and the like. Such assays for inhibitors and activators include, e.g., expressing the subject taste cell specific protein in vitro, in cells, cell extracts, or cell membranes, applying putative modulator compounds, and then determining the functional effects on activity, as described above. “Modulators” include any compound that directly modulates the activity of a protein, herein GPR113 or in association with another compound that binds or modulates the activity of the protein, e.g., GPR113. As mentioned GPR113 may be expressed alone or in association with another GPCR such as T1R3, GPR40, GPR120 or TRPM5.

Samples or assays comprising the proteins encoded by genes identified herein that are treated with a potential activator, inhibitor, or modulator are compared to control samples without the inhibitor, activator, or modulator to examine the extent of activation. Control samples (untreated with inhibitors) are assigned a relative protein activity value of 100%. Inhibition of a receptor is achieved when the activity value relative to the control is about 80%, preferably 50%, more preferably 25-0%. Activation of a receptor is achieved when the activity value relative to the control (untreated with activators) is 110%, more preferably 150%, more preferably 200-500% (i.e., two to five fold higher relative to the control), more preferably 1000-3000% or higher.

The term “test compound” or “drug candidate” or “modulator” or grammatical equivalents as used herein describes any molecule, either naturally occurring or synthetic compound, preferably a small molecule, or a protein, oligopeptide (e.g., from about 5 to about 25 amino acids in length, preferably from about 10 to 20 or 12 to 18 amino acids in length, preferably 12, 15, or 18 amino acids in length), small organic molecule, polysaccharide, lipid, fatty acid, polynucleotide, siRNA, miRNA, oligonucleotide, ribozyme, etc., to be tested for the capacity to modulate fatty acid, fat or lipid sensation. The test compound can be in the form of a library of test compounds, such as a combinatorial or randomized library that provides a sufficient range of diversity. Test compounds are optionally linked to a fusion partner, e.g., targeting compounds, rescue compounds, dimerization compounds, stabilizing compounds, addressable compounds, and other functional moieties. Conventionally, new chemical entities with useful properties are generated by identifying a test compound (called a “lead compound”) with some desirable property or activity, e.g., inhibiting activity, creating variants of the lead compound, and evaluating the property and activity of those variant compounds. Often, high throughput screening (HTS) methods are employed for such an analysis.

A “small organic molecule” refers to an organic molecule, either naturally occurring or synthetic, that has a molecular weight of more than about 50 daltons and less than about 2500 daltons, preferably less than about 2000 daltons, preferably between about 100 to about 1000 daltons, more preferably between about 200 to about 500 daltons.

“Biological sample” include sections of tissues such as biopsy and autopsy samples, and frozen sections taken for histologic purposes. Such samples include blood, sputum, tissue, cultured cells, e.g., primary cultures, explants, and transformed cells, stool, urine, etc. A biological sample is typically obtained from a eukaryotic organism, most preferably a mammal such as a primate e.g., chimpanzee or human; cow; dog; cat; a rodent, e.g., guinea pig, rat, mouse; rabbit; or a bird; reptile; or fish.

The terms “identical” or percent “identity,” in the context of two or more nucleic acids or polypeptide sequences, refer to two or more sequences or subsequences that are the same or have a specified percentage of amino acid residues or nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region (e.g., a gene or sequence contained herein), when compared and aligned for maximum correspondence over a comparison window or designated region) as measured using a BLAST or BLAST 2.0 sequence comparison algorithms with default parameters described below, or by manual alignment and visual inspection (see, e.g., NCBI web site or the like). Such sequences are then said to be “substantially identical.” This definition also refers to, or may be applied to, the compliment of a test sequence. The definition also includes sequences that have deletions and/or additions, as well as those that have substitutions. As described below, the preferred algorithms can account for gaps and the like. Preferably, identity exists over a region that is at least about 25 amino acids or nucleotides in length, or more preferably over a region that is 50-100 amino acids or nucleotides in length.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Preferably, default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Current Protocols in Molecular Biology (Ausubel et al., eds. 1995 supplement)).

A preferred example of algorithm that is suitable for determining percent sequence identity and sequence similarity are the BLAST and BLAST 2.0 algorithms, which are described in Altschul et al., Nucl. Acids Res. 25:3389-3402 (1977) and Altschul et al., J. Mol. Biol. 215:403-410 (1990), respectively. BLAST and BLAST 2.0 are used, with the parameters described herein, to determine percent sequence identity for the nucleic acids and proteins of the invention. Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always>0) and N (penalty score for mismatching residues; always<0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word length (W) of 11, an expectation (E) of 10, M=5, N=−4 and a comparison of both strands. For amino acid sequences, the BLASTP program uses as defaults a word length of 3, and expectation (E) of 10, and the BLOSUM62 scoring matrix (see Henikoff & Henikoff, Proc. Natl. Acad. Sci., USA 89:10915 (1989)) alignments (B) of 50, expectation (E) of 10, M=5, N=−4, and a comparison of both strands.

“Nucleic acid” refers to deoxyribonucleotides or ribonucleotides and polymers thereof in either single- or double-stranded form, and complements thereof. The term encompasses nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, 2-O-methyl ribonucleotides, peptide-nucleic acids (PNAs).

Unless otherwise indicated, a particular nucleic acid sequence also implicitly encompasses conservatively modified variants thereof (e.g., degenerate codon substitutions) and complementary sequences, as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); Rossolini et al., Mol. Cell. Probes 8:91-98 (1994)). The term nucleic acid is used interchangeably with gene, cDNA, mRNA, oligonucleotide, and polynucleotide.

A particular nucleic acid sequence also implicitly encompasses “splice variants.” Similarly, a particular protein encoded by a nucleic acid implicitly encompasses any protein encoded by a splice variant of that nucleic acid. “Splice variants,” as the name suggests, are products of alternative splicing of a gene. After transcription, an initial nucleic acid transcript may be spliced such that different (alternate) nucleic acid splice products encode different polypeptides. Mechanisms for the production of splice variants vary, but include alternate splicing of exons. Alternate polypeptides derived from the same nucleic acid by read-through transcription are also encompassed by this definition. Any products of a splicing reaction, including recombinant forms of the splice products, are included in this definition. An example of potassium channel splice variants is discussed in Leicher, et al., J. Biol. Chem. 273(52):35095-35101 (1998).

The terms “polypeptide,” “peptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

“Conservatively modified variants” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence with respect to the expression product, but not with respect to actual probe sequences.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention.

The following eight groups each contain amino acids that are conservative substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and 8) Cysteine (C), Methionine (M) (see, e.g., Creighton, Proteins (1984)).

Macromolecular structures such as polypeptide structures can be described in terms of various levels of organization. For a general discussion of this organization, see, e.g., Alberts et al., Molecular Biology of the Cell (3rd ed., 1994) and Cantor and Schimmel, Biophysical Chemistry Part I: The Conformation of Biological Macromolecules (1980). “Primary structure” refers to the amino acid sequence of a particular peptide. “Secondary structure” refers to locally ordered, three dimensional structures within a polypeptide. These structures are commonly known as domains, e.g., transmembrane domains, pore domains, and cytoplasmic tail domains. Domains are portions of a polypeptide that form a compact unit of the polypeptide and are typically 15 to 350 amino acids long. Exemplary domains include extracellular domains, transmembrane domains, and cytoplasmic domains. Typical domains are made up of sections of lesser organization such as stretches of β sheet and α-helices. “Tertiary structure” refers to the complete three dimensional structure of a polypeptide monomer. “Quaternary structure” refers to the three dimensional structure formed by the noncovalent association of independent tertiary units. Anisotropic terms are also known as energy terms.

A “label” or a “detectable moiety” is a composition detectable by spectroscopic, photochemical, biochemical, immunochemical, chemical, or other physical means. For example, useful labels include 3²p, fluorescent dyes, electron-dense reagents, enzymes (e.g., as commonly used in an ELISA), biotin, digoxigenin, or haptens and proteins which can be made detectable, e.g., by incorporating a radiolabel into the peptide or used to detect antibodies specifically reactive with the peptide.

The term “recombinant” when used with reference, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Thus, for example, recombinant cells express genes that are not found within the native (non-recombinant) form of the cell or express native genes that are otherwise abnormally expressed, under expressed or not expressed at all.

The term “heterologous” when used with reference to portions of a nucleic acid indicates that the nucleic acid comprises two or more subsequences that are not found in the same relationship to each other in nature. For instance, the nucleic acid is typically recombinantly produced, having two or more sequences from unrelated genes arranged to make a new functional nucleic acid, e.g., a promoter from one source and a coding region from another source. Similarly, a heterologous protein indicates that the protein comprises two or more subsequences that are not found in the same relationship to each other in nature (e.g., a fusion protein).

The phrase “stringent hybridization conditions” refers to conditions under which a probe will hybridize to its target subsequence, typically in a complex mixture of nucleic acids, but to no other sequences. Stringent conditions are sequence-dependent and will be different in different circumstances. Longer sequences hybridize specifically at higher temperatures. An extensive guide to the hybridization of nucleic acids is found in Tijssen, Techniques in Biochemistry and Molecular Biology-Hybridization with Nucleic Probes, “Overview of principles of hybridization and the strategy of nucleic acid assays” (1993). Generally, stringent conditions are selected to be about 5-10° C. lower than the thermal melting point (Tₘ) for the specific sequence at a defined ionic strength pH. The Tₘ is the temperature (under defined ionic strength, pH, and nucleic concentration) at which 50% of the probes complementary to the target hybridize to the target sequence at equilibrium (as the target sequences are present in excess, at Tₘ, 50% of the probes are occupied at equilibrium). Stringent conditions may also be achieved with the addition of destabilizing agents such as formamide. For selective or specific hybridization, a positive signal is at least two times background, preferably 10 times background hybridization. Exemplary stringent hybridization conditions can be as following: 50% formamide, 5×SSC, and 1% SDS, incubating at 42° C., or, 5×SSC, 1% SDS, incubating at 65° C., with wash in 0.2×SSC, and 0.1% SDS at 65° C.

Nucleic acids that do not hybridize to each other under stringent conditions are still substantially identical if the polypeptides which they encode are substantially identical. This occurs, for example, when a copy of a nucleic acid is created using the maximum codon degeneracy permitted by the genetic code. In such cases, the nucleic acids typically hybridize under moderately stringent hybridization conditions. Exemplary “moderately stringent hybridization conditions” include a hybridization in a buffer of 40% formamide, 1 M NaCl, 1% SDS at 37° C., and a wash in 1×SSC at 45° C. A positive hybridization is at least twice background. Those of ordinary skill will readily recognize that alternative hybridization and wash conditions can be utilized to provide conditions of similar stringency. Additional guidelines for determining hybridization parameters are provided in numerous reference, e.g., and Current Protocols in Molecular Biology, ed. Ausubel, et al.

For PCR, a temperature of about 36° C. is typical for low stringency amplification, although annealing temperatures may vary between about 32° C. and 48° C. depending on primer length. For high stringency PCR amplification, a temperature of about 62° C. is typical, although high stringency annealing temperatures can range from about 50° C. to about 65° C., depending on the primer length and specificity. Typical cycle conditions for both high and low stringency amplifications include a denaturation phase of 90° C.-95° C. for 30 sec-2 min., an annealing phase lasting 30 sec.-2 min., and an extension phase of about 72° C. for 1-2 min. Protocols and guidelines for low and high stringency amplification reactions are provided, e.g., in Innis et al. (1990) PCR Protocols, A Guide to Methods and Applications, Academic Press, Inc. N.Y.).

“Antibody” refers to a polypeptide comprising a framework region from an immunoglobulin gene or fragments thereof that specifically binds and recognizes an antigen. The recognized immunoglobulin genes include the κ, λ, α, γ, δ, ε, and μ constant region genes, as well as the myriad immunoglobulin variable region genes. Light chains are classified as either kappa or lambda. Heavy chains are classified as γ, μ, α, δ, or ε, which in turn define the immunoglobulin classes, IgG, IgM, IgA, IgD and IgE, respectively. Typically, the antigen-binding region of an antibody will be most critical in specificity and affinity of binding.

The term antibody, as used herein, also includes antibody fragments either produced by the modification of whole antibodies, or those synthesized de novo using recombinant DNA methodologies (e.g., single chain Fv), chimeric, humanized or those identified using phage display libraries (see, e.g., McCafferty et al., Nature 348:552-555 (1990)) For preparation of antibodies, e.g., recombinant, monoclonal, or polyclonal antibodies, many technique known in the art can be used (see, e.g., Kohler & Milstein, Nature 256:495-497 (1975); Kozbor et al., Immunology Today 4: 72 (1983); Cole et al., pp. 77-96 in Monoclonal Antibodies and Cancer Therapy, Alan R. Liss, Inc. (1985); Coligan, Current Protocols in Immunology (1991); Harlow & Lane, Antibodies, A Laboratory Manual (1988) and Harlow & Lane, Using Antibodies, A Laboratory Manual (1999); and Goding, Monoclonal Antibodies: Principles and Practice (2d ed. 1986)).

The phrase “specifically (or selectively) binds” to an antibody or “specifically (or selectively) immunoreactive with,” when referring to a protein or peptide, refers to a binding reaction that is determinative of the presence of the protein, often in a heterogeneous population of proteins and other biologics. Thus, under designated immunoassay conditions, the specified antibodies bind to a particular protein at least two times the background and more typically more than 10 to 100 times background. Specific binding to an antibody under such conditions requires an antibody that is selected for its specificity for a particular protein. For example, polyclonal antibodies raised to a protein, polymorphic variants, alleles, orthologs, and conservatively modified variants, or splice variants, or portions thereof, can be selected to obtain only those polyclonal antibodies that are specifically immunoreactive with proteins and not with other proteins. This selection may be achieved by subtracting out antibodies that cross-react with other molecules. A variety of immunoassay formats may be used to select antibodies specifically immunoreactive with a particular protein. For example, solid-phase ELISA immunoassays are routinely used to select antibodies specifically immunoreactive with a protein (see, e.g., Harlow & Lane, Antibodies, A Laboratory Manual (1988) for a description of immunoassay formats and conditions that can be used to determine specific immunoreactivity).

By “therapeutically effective dose” herein is meant a dose that produces effects for which it is administered. The exact dose will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); and Pickar, Dosage Calculations (1999)).

Having provided the foregoing definitions, the invention is now described in detail.

As described in the experimental example infra, experiments conducted by the inventors have revealed that GPR113 encodes a GPR polypeptide that detects fat tastants. This gene was initially identified as being a taste specific gene because it was not expressed in the sampled non-taste cells (lingual epithelium; LE) and was expressed in significantly lesser amounts in the sample of primate taste bud cells containing cells obtained from the bottom half of the taste buds. This was quantified by TaqMan in laser capture microdissection (LCM) derived cDNA from both LE and TB from the same donors. The GRP113 gene was determined to be expressed in human TB but not in LE and based thereon considered to be a taste-specific gene. GPR113 is expressed in taste cells that express TRPM5, a key taste signal transduction protein, and is specifically expressed in a subset of taste cells which also express T1R3.

As described infra, it has been shown that mice lacking a functional GPR113 gene (GPR113 knockout mice) have diminished preference for and intake of certain fats and fatty acids. By contrast, the response of these mice to other types of tastants is unaffected.

Because GPR113 has been shown to encode a functional fat taste receptor this receptor and cells which express same may be utilized as a screening tool for identifying compounds that mimic fat taste or which regulate fat taste perception or fat absorption and metabolism. Such compounds can be incorporated into foods as fat replacers or to modulate fat taste perception or in medicaments or comestibles to modulate fat metabolism and regulate dietary fat consumption and body weight control.

GPR113 was identified as potentially being involved in taste or another taste cell function based, in part, on its expression in taste tissue. Using immunochemical staining techniques, the inventors have found that GPR113 is expressed at relatively high levels in the CV taste buds of mice, primates and humans with little or no detectable expression in lingual epithelium. Using quantitative polymerase chain reaction (qPCR) it was demonstrated that GPR113 is expressed at relatively high levels in the CV taste buds of mice, primates and humans with little or no detectable expression in lingual epithelium. Using in situ hybridization (ISH) it was further demonstrated that GPR113 KO mice have no visible expression of GPR113 mRNA in CV.

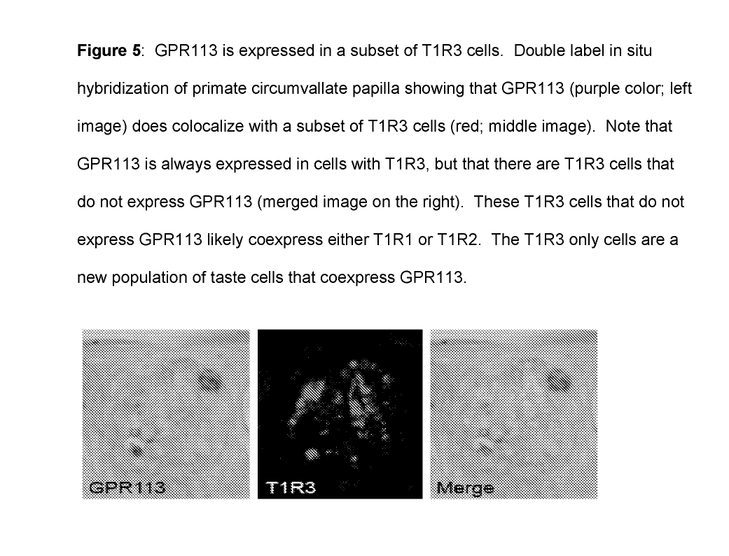

Further histological characterization of GPR113 in wild-type taste tissue revealed that a subset of cells that express T1R3 express GPR113, however there is no overlap with cells expressing T1R2, T1R1 or T2Rs such as T2R05. As shown in FIG. 5, double label in situ hybridization of primate circumvallate papilla shows that GPR113 is always expressed in cells with T1R3; however, T1R3 cells do not always express GPR113. The T1R3 cells that do not express GPR113 include those which co-express either T1R1 or T1R2.

It was theorized based on this co-expression that T1R3 cells which express GPR113 constitute a new population of taste cells. This hypothesis was further based on the observation that GPR113 expression overlaps with TRPM5 expression in a subset of cells but there is no overlap with cell populations expressing PKD2L1 or α-gustducin. This profile of GPR113 expression suggested to the inventors that GPR113 may modulate a different taste modality. In fact, as shown herein it modulates fat or lipid taste cell function and responses to fat and lipid tastants.

Standard immunochemical staining and co-localization studies carried out with TRPM5, corroborate that GPR113 expressing cells express TRPM5. Because GPR113 cells express TRPM5, it was hypothesized that this receptor likely utilizes a common transduction pathway as the pathway used by other GPCRs involved in sweet, bitter and umami taste.

Behavioral tests in knockout mice described infra have shown that GPR113 functions in sensory perception of fat taste. Mice lacking a functional GPR113 receptor were given the choice between two drinking bottles, one containing a fat and one containing vehicle only, as describe in the examples infra. The GPR113 KO mice have impaired responsiveness to a variety of different fat stimuli (soybean oil, sefa soyate oil, intralipid).

Additionally, brief access licking paradigms that rely more on taste processes and limit post-ingestive influence show that wild-type mice exhibit increased licking with increasing concentrations of oil stimuli (soybean oil, corn oil, sefa soyate, linoleic acid, oleic acid), whereas this preference is significantly attenuated in GPR113 KO mice. These findings suggest that GPR113 is necessary for normal responsiveness to fats such as soybean oil and corn oil as well as fatty acids such as linoleic acid and oleic acid. Moreover, compared with normal mice, the GPR113 knockout animals consumed less fat.

By contrast, GPR113 knockout animals also showed no preference for a non-nutritive oil (mineral oil) indicating that the effect on fat consumption was a function of fat taste and not because of other attributes of the tested fats such as viscosity or mouth feel. The fat specificity of GPR113 was further established based on the fact that there was no difference in the responsiveness of wild-type and knockout animals to sweet, bitter, salty and sour tastants.

Also, licking profiles from wild-type mice with glossopharyngeal nerve transection (GLX) mimic that of GPR113 knockout (GPR113 KO) mice. Further, GLX mice relative to their sham transected counterparts have decreased licking responses to soybean oil but not sucrose. Together these results indicate that GPR113 is a taste receptor that specifically responds to fat, lipid and fatty acid compounds and is involved in regulating fat, lipid and/or fatty acid associated taste.

More specifically, in order to further validate the role of the subject gene as a fat taste receptor, transgenic mice were created wherein expression of this gene was knocked out. Behavioral (2-bottle preference tests and brief access licking tests) experiments were performed to determine if the animals are deficient in or lack fat taste perception.

As reported in the examples, the GPR113 gene knockout mice, relative to the wild-type mice, had reduced responsiveness to different fats and oils including different soybean oil and corn oil compositions as well as to the fatty acids linoleic acid and oleic acid. By contrast, the knockout and wild-type mice showed no difference in taste responsiveness to other (non-fat) tastants (sweet, bitter, salt, sour) such as polycose, sucrose, NaCl, KC, citric acid and quinine. In addition there was no difference in responsiveness to a tasteless oil, mineral oil, confirming that the responsiveness of GPR113 to different fats and its modulatory effect on fat intake is taste specific, i.e., it is not a function of viscosity or “mouth-feel”.

Based thereon this taste receptor and cells which express GPR113, both recombinant and endogenous taste cells, may be used in screens, e.g., high-throughput screens in order to identify enhancers and blockers of fat taste as well as compounds that mimic fat taste. Also, the effects of the identified compounds on fat taste may be verified in human or animal taste tests, i.e., to determine if the identified compounds augment or repress fat taste perception or elicit a fatty taste.

Therefore the present invention includes the use of cell-based assays to identify fat taste modulators (e.g., agonists, antagonists, enhancers, blockers) using endogenous or recombinant cells which express GPR113 polypeptides. These cells may also express T1R3 and/or TRPM5. These compounds have potential application in modulating human taste perception to different fats, oils, lipids and fatty acids and may affect other fat related physiological functions including fat absorption and metabolism, or the hedonic response to fats as it relates to dietary control and preference

Compounds identified in screening assays, e.g., electrophysiological assays, FFRET assays and their biologically acceptable derivatives are to be tested in human taste tests using human volunteers to confirm their effect on fat taste perception. In addition compounds identified as potential therapeutics for modulating fat absorption or metabolism will be evaluated in appropriate in vitro and in vivo models depending on the nature of the intended application. For example compounds identified as potential therapeutics for treating diabetes or obesity may be evaluated in well-known diabetic or obesity animal models such the db/db mouse, Zucker fatty rat, ZDF rat, and diet-induced obese rodent models. Similarly, compounds identified as potential therapeutics potentially may be used to treat Irritable Bowel Syndrome (IBS) or Crohn's disease, gall bladder related diseases or syndromes, or liver diseases and other diseases involving aberrant fat metabolism. The efficacy of these compounds as putative therapeutics may be tested in appropriate in vitro or animal models for the particular disease or condition.

As discussed further infra, the cell-based assays used to identify fat taste modulatory or therapeutic compounds will preferably comprise high throughput screening platforms to identify compounds that modulate (e.g., agonize, antagonize, block or enhance) the activity of GPR113 using cells that express the GPR113 gene disclosed herein optionally with other taste specific genes or combinations thereof. Additionally, these sequences may be modified to introduce silent mutations or mutations having a functional effect such as defined mutations that affection (sodium) influx. The assays may comprise fluorometric or electrophysiological assays effected in amphibian oocytes or assays using mammalian cells that express the subject GPCR. Also, compounds that modulate GPR113 putatively involved in taste may be detected by ion flux assays, e.g., radiolabeled-ion flux assays or atomic absorption spectroscopic coupled ion flux assays or label-free optical biosensor assays. As disclosed supra, these compounds have potential application in modulating human fat taste perception or for modulating other biological processes involving fat absorption and metabolism and diseases such as autoimmune disorders involving aberrant fat metabolism or elimination.

The subject cell-based assays may use wild-type or mutant nucleic acid sequences which are expressed in desired cells, such as oocytes, insect or human cells such as CHO, COS, BHK, STO or other human or mammalian cells conventionally used in screens for GPCR modulatory compounds. These cells may further be engineered to express other sequences, e.g., other taste GPCRs, e.g., T1Rs or T2Rs such as T1R3 as well as appropriate G proteins and/or taste specific ion channels such as TRPM5 or TRPM8. The oocyte system is advantageous as it allows for direct injection of multiple mRNA species, provides for high protein expression and can accommodate the deleterious effects inherent in the overexpression of ion channels. The drawbacks however are that electrophysiological screening using amphibian oocytes is not as amenable to high throughput screening of large numbers of compounds and is not a mammalian system. As noted, the present invention embraces assays using mammalian cells, preferably high throughput assays.

In an exemplary embodiment high throughput screening assays are effected using mammalian cells transfected or seeded into wells or culture plates wherein functional expression in the presence of test compounds is allowed to proceed and activity is detected using calcium, membrane-potential fluorescent or ion (sodium) fluorescent dyes. However, as described infra this fluorescent assay is exemplary of assay methods for identifying compounds that modulate GPR113 function and the invention embraces non-fluorescent assay methods.

The invention specifically provides methods of screening for modulators, e.g., agonists, antagonists, activators, inhibitors, blockers, stimulators, enhancers, etc., of human fat taste and taste sensation (intensity) and potential therapeutics that target other taste cell functions or phenotypes using the nucleic acids and proteins, sequences provided herein. Such modulators can affect fat taste and taste cell related functions and phenotypes, e.g., by modulating transcription, translation, mRNA or protein stability; by altering the interaction of the polypeptide with the plasma membrane, or other molecules; or by affecting GPR113 protein activity.

Compounds are screened, e.g., using high throughput screening (HTS), to identify those compounds that can bind to and/or modulate the activity of the subject fat taste receptor or fragment thereof. In the present invention, the subject GPR113 proteins alone or when expressed in association with T1R3 and/or TRPM5 are recombinantly or endogenously expressed by cells used for screening, e.g., human cells, other mammalian cells, or frog oocytes and the modulation of activity is assayed by using any measure of GPCR function, such as binding assays, conformational assays, calcium based assays, measurement of the membrane potential, measures of changes in intracellular sodium or lithium levels, or optical biosensor changes. More specifically, the assays may use human, non-human primate or other mammalian cells which endogenously express one or more of GPR113, TRPM5 and T1R3. These cells may further endogenously express a G protein or a nucleic acid may be introduced therein encoding a G protein such as Ga15, Ga16, transducin or gustducin or a chimera of any of the foregoing such as Ga15 or Ga16/gust44 or Ga15 or Ga16/transducin44 wherein the C-terminal 44 amino acids of Ga15 or Ga16 are substituted for the corresponding 44 amino acids of gustducin or transducin.

Methods of assaying ion, e.g., cation, channel function include, for example, patch clamp techniques, two electrode voltage clamping, measurement of whole cell currents, and fluorescent imaging techniques that use ion⁻ sensitive fluorescent dyes and ion flux assays, e.g., radiolabeled-ion flux assays or ion flux assays. Other assays are exemplified infra.

An enhancer or activator of GPR113 or a compound that specifically binds GPR113 identified according to the current application can be used for a number of different purposes. For example, it can be included as a flavoring agent to modulate enhance) the taste of foods, beverages, soups, medicines, and other products containing a fat, oil, lipid, or fatty acid which is for human consumption. Additionally, the invention provides kits for carrying out the herein-disclosed assays. Compounds identified using these assays that specifically bind or modulate the activity of GPR113 alone or when GPR113 is expressed in association with T1R3 and/or TRPM5, e.g., enhancers or activators, may also be used to modulate fat metabolism and diet control as discussed previously.

Also as noted previously the present invention particularly provides the use of the subject taste specific gene as a marker which can be used to enrich, identify or isolate specific taste cell subsets or to enrich, identify or isolate fat taste bud committed stem cells and/or cells that modulate fat metabolism and diet control.

### Recombinant Expression of Taste Gene Identified Herein

To obtain high level expression of a cloned gene, such as those cDNAs encoding the subject GPR113 gene, one typically subclones the gene into an expression vector that contains a strong promoter to direct transcription, a transcription/translation terminator, and if for a nucleic acid encoding a protein, a ribosome binding site for translational initiation. Suitable eukaryotic and prokaryotic promoters are well known in the art and described, e.g., in Sambrook et al., and Ausubel et al., supra. For example, bacterial expression systems for expressing the taste specific protein are available in, e.g., E. coli, Bacillus sp., and Salmonella (Palva et al., Gene 22:229-235 (1983); Mosbach et al., Nature 302:553-555 (1983). Kits for such expression systems are commercially available. Eukaryotic expression systems for mammalian cells, yeast, and insect cells are well known in the art and are also commercially available. For example, retroviral expression systems may be used in the present invention. As described infra, the subject taste affecting genes are preferably expressed in human or non-human primate or other mammalian cells such as, COS, CHO, BHK and the like which are widely used for high throughput screening.

Selection of the promoter used to direct expression of a heterologous nucleic acid depends on the particular application. The promoter is preferably positioned about the same distance from the heterologous transcription start site as it is from the transcription start site in its natural setting. As is known in the art, however, some variation in this distance can be accommodated without loss of promoter function.

In addition to the promoter, the expression vector typically contains a transcription unit or expression cassette that contains all the additional elements required for the expression of the nucleic acid in host cells. A typical expression cassette thus contains a promoter operably linked to the nucleic acid sequence encoding the identified gene and signals required for efficient polyadenylation of the transcript, ribosome binding sites, and translation termination. Additional elements of the cassette may include enhancers and, if genomic DNA is used as the structural gene, introns with functional splice donor and acceptor sites.

In addition to a promoter sequence, the expression cassette should also contain a transcription termination region downstream of the structural gene to provide for efficient termination. The termination region may be obtained from the same gene as the promoter sequence or may be obtained from different genes.

The particular expression vector used to transport the genetic information into the cell is not particularly critical. Any of the conventional vectors used for expression in eukaryotic or prokaryotic cells may be used. Standard bacterial expression vectors include plasmids such as pBR322 based plasmids, pSKF, pET23D, and fusion expression systems such as MBP, GST, and LacZ. Epitope tags can also be added to recombinant proteins to provide convenient methods of isolation, e.g., c-myc. Sequence tags may be included in an expression cassette for nucleic acid rescue. Markers such as fluorescent proteins, green or red fluorescent protein, 8-gal, CAT, and the like can be included in the vectors as markers for vector transduction.

Expression vectors containing regulatory elements from eukaryotic viruses are typically used in eukaryotic expression vectors, e.g., SV40 vectors, papilloma virus vectors, retroviral vectors, and vectors derived from Epstein-Barr virus. Other exemplary eukaryotic vectors include pMSG, pAV009/A⁺, pMTO10/A⁺, pMAMneo-5, baculovirus pDSVE, and any other vector allowing expression of proteins under the direction of the CMV promoter, SV40 early promoter, SV40 later promoter, metallothionein promoter, murine mammary tumor virus promoter, Rous sarcoma virus promoter, polyhedrin promoter, or other promoters shown effective for expression in eukaryotic cells.

Expression of proteins from eukaryotic vectors can also be regulated using inducible promoters. With inducible promoters, expression levels are tied to the concentration of inducing agents, such as tetracycline or ecdysone, by the incorporation of response elements for these agents into the promoter. Generally, high level expression is obtained from inducible promoters only in the presence of the inducing agent; basal expression levels are minimal.

The vectors used in the invention may include a regulatable promoter, e.g., tet-regulated systems and the RU-486 system (see, e.g., Gossen & Bujard, Proc. Nat'l Acad. Sci USA 89:5557 (1992); Oligino et al., Gene Ther. 5:491-496 (1998); Wang et al., Gene Ther. 4:432-441 (1997); Neering et al., Blood 88:1 157-1155 (1996); and Rendahl et al., Nat. Biotechnol. 16:757-761 (1998)). These impart small molecule control on the expression of the candidate target nucleic acids. This beneficial feature can be used to determine that a desired phenotype is caused by a transfected cDNA rather than a somatic mutation.

Some expression systems have markers that provide gene amplification such as thymidine kinase and dihydrofolate reductase. Alternatively, high yield expression systems not involving gene amplification are also suitable, such as using a baculovirus vector in insect cells, with a gene sequence under the direction of the polyhedrin promoter or other strong baculovirus promoters.

The elements that are typically included in expression vectors also include a replicon that functions in the particular host cell. In the case of E. coli, the vector may contain a gene encoding antibiotic resistance to permit selection of bacteria that harbor recombinant plasmids, and unique restriction sites in nonessential regions of the plasmid to allow insertion of eukaryotic sequences. The particular antibiotic resistance gene chosen is not critical, any of the many resistance genes known in the art are suitable. The prokaryotic sequences are preferably chosen such that they do not interfere with the replication of the DNA in eukaryotic cells, if necessary.

Standard transfection methods may be used to produce bacterial, mammalian, yeast or insect cell lines that express large quantities of the desired taste specific protein, which are then purified using standard techniques (see, e.g., Colley et al., J. Biol. Chem. 264:17619-17622 (1989); Guide to Protein Purification, in Methods in Enzymology, vol. 182 (Deutscher, ed., 1990)). Transformation of eukaryotic and prokaryotic cells are performed according to standard techniques (see, e.g., Morrison, J. Bact. 132:349-351 (1977); Clark-Curtiss & Curtiss, Methods in Enzymology 101:347-362 (Wu et al., eds, 1983). Any of the well-known procedures for introducing foreign nucleotide sequences into host cells may be used. These include the use of calcium phosphate transfection, polybrene, protoplast fusion, electroporation, biolistics, liposomes, microinjection, plasma vectors, viral vectors and any of the other well-known methods for introducing cloned genomic DNA, cDNA, synthetic DNA or other foreign genetic material into a host cell (see, e.g., Sambrook et al., supra). It is only necessary that the particular genetic engineering procedure used be capable of successfully introducing at least one gene into the host cell capable of expressing the gene.

After the expression vector is introduced into the cells, the transfected cells are cultured under conditions favoring expression of the gene. In some instances, such polypeptides may be recovered from the culture using standard techniques identified below.

### Assays for Identifying GPR113 (Fat Taste) Modulators (Agonists, Antagonist, Blockers, Enhancers, Activators)

### Detection of GPR113 Modulators

Compositions and methods for determining whether a test compound specifically binds to a GPR113 receptor of the invention, both in vitro and in vivo, are described below. Many aspects of cell physiology can be monitored to assess the effect of ligand binding to a GPR113 polypeptide of the invention. These assays may be performed on intact cells expressing GPR113 receptor, on permeabilized cells, or on membrane fractions produced by standard methods or in vitro de novo synthesized proteins.

In vivo, taste receptors bind tastants and initiate the transduction of chemical stimuli into electrical signals. An activated or inhibited G protein will in turn alter the properties of target enzymes, channels, and other effector proteins. Some examples are the activation of cGMP phosphodiesterase by transducin in the visual system, adenylate cyclase by the stimulatory G protein, phospholipase C by Gq and other cognate G proteins, and modulation of diverse channels by Gi and other G proteins. Downstream consequences can also be examined such as generation of diacyl glycerol and IP3 by phospholipase C, and in turn, for calcium mobilization by IP3.

The GPR113 proteins or polypeptides of the assay will preferably be selected from a polypeptide having the polypeptide sequence selected from those disclosed herein or fragments or conservatively modified variants thereof. As noted the assays may utilize GPR113 polypeptides which are isolated from a cell or produced via recombinant methods or the assays may use cells that endogenously or recombinantly express GPR113 and optionally further express T1R3 and/or TRPM5. Optionally, the fragments and variants used in these assays can be antigenic fragments and variants which bind to an anti-GPR113 antibody such as fragments containing the extracellular or transmembrane domains thereof. Further optionally, the fragments and variants can bind to or are activated by one or more fats, oils, fatty acids or lipids.

Alternatively, the GPR113 proteins or polypeptides of the assay can be derived from a eukaryotic host cell and can include an amino acid subsequence having amino acid sequence identity to the GPR113 polypeptides disclosed herein, or fragments or conservatively modified variants thereof. Generally, the amino acid sequence identity will be at least 35 to 50%, or optionally 75%, 85%, 90%, 95%, 96%, 97%, 98%, or 99%. Optionally, the GPR113 proteins or polypeptides of the assays can comprise a domain of a GPR113 protein, such as an extracellular domain, transmembrane region, transmembrane domain, cytoplasmic domain, ligand-binding domain, and the like. Further, as described above, the GPR113 protein or a domain thereof can be covalently linked to a heterologous protein to create a chimeric protein used in the assays described herein.

Compounds that themselves bind GPR113 or which modulate, elicit, agonize, antagonize, or block GPR113 receptor activity or which modulate, elicit, agonize, antagonize, or block GPR113 receptor activation or binding by other ligands such as fats, oils, fatty acids and lipids are tested using GPR113 proteins or polypeptides as described above, either recombinant or naturally occurring. The GPR113 proteins or polypeptides can be isolated, expressed in a cell, expressed in a membrane derived from a cell, expressed in tissue or in an animal, either recombinant or naturally occurring. For example, tongue slices, dissociated cells from a tongue, transformed cells, or membranes can be used. Whether a compound elicits such an effect on GPR113 receptor activity or specifically binds or affects the binding of another compound to the GPR113 receptor can be tested using one of the in vitro or in vivo assays described herein. In addition, the effects of these identified compounds in human or other animal taste tests may be affected.

### 1. In Vitro Binding Assays

Taste transduction can also be examined in vitro with soluble or solid state reactions, using the GPR113 polypeptides of the invention. In a particular embodiment, GPR113 ligand-binding domains can be used in vitro in soluble or solid state reactions to assay for ligand binding.

For instance, the GPR113 N-terminal domain is predicted to be involved in ligand binding. More particularly, GPR113 belongs to a GPCR sub-family that is characterized by large, approximately 600 amino acid, extracellular N-terminal segments. These N-terminal segments are thought to form the ligand-binding domains, and are therefore useful in biochemical assays to identify GPR113 agonists and antagonists. It is possible that the ligand-binding domain may be formed by additional portions of the extracellular domain, such as the extracellular loops of the transmembrane domain, or portions of the transmembrane domain.

Ligand binding to GPR113 polypeptides of the invention can be tested in solution, in a bilayer membrane, optionally attached to a solid phase, in a lipid monolayer, or in vesicles. Binding of a compound to GPR113 can be tested by various methods e.g., by detecting changes in spectroscopic characteristics (e.g., fluorescence, absorbance, refractive index) hydrodynamic (e.g., shape), chromatographic, or solubility properties.

In another embodiment of the invention, a GTP γ³⁵S assay may be used. As described above, upon activation of a GPCR, the G_{α} subunit of the G protein complex is stimulated to exchange bound GDP for GTP. Ligand-mediated stimulation of G protein exchange activity can be measured in a biochemical assay measuring the binding of added radioactively labeled GTP γ³⁵S to the G protein in the presence of a putative ligand. Typically, membranes containing the chemosensory receptor of interest are mixed with a complex of G proteins. Potential inhibitors and/or activators and GTP γ³⁵S are added to the assay, and binding of GTP γ³⁵S to the G protein is measured. Binding can be measured by liquid scintillation counting or by any other means known in the art, including scintillation proximity assays (SPA). In other assays formats, fluorescently labeled GTPγS can be utilized.

### 2. Fluorescence Polarization Assays

In another embodiment, Fluorescence Polarization (“FP”) based assays may be used to detect and monitor ligand binding. Fluorescence polarization is a versatile laboratory technique for measuring equilibrium binding, nucleic acid hybridization, and enzymatic activity. Fluorescence polarization assays are homogeneous in that they do not require a separation step such as centrifugation, filtration, chromatography, precipitation, or electrophoresis. These assays are done in real time, directly in solution and do not require an immobilized phase. Polarization values can be measured repeatedly and after the addition of reagents since measuring the polarization is rapid and does not destroy the sample. Generally, this technique can be used to measure polarization values of fluorophores from low picomolar to micromolar levels. This section describes how fluorescence polarization can be used in a simple and quantitative way to measure the binding of ligands to the GPR113 polypeptides of the invention.

When a fluorescently labeled molecule is excited with plane-polarized light, it emits light that has a degree of polarization that is inversely proportional to its molecular rotation. Large fluorescently labeled molecules remain relatively stationary during the excited state (4 nanoseconds in the case of fluorescein) and the polarization of the light remains relatively constant between excitation and emission. Small fluorescently labeled molecules rotate rapidly during the excited state and the polarization changes significantly between excitation and emission. Therefore, small molecules have low polarization values and large molecules have high polarization values. For example, a single-stranded fluorescein-labeled oligonucleotide has a relatively low polarization value but when it is hybridized to a complementary strand, it has a higher polarization value. When using FP to detect and monitor tastant-binding which may activate or inhibit the chemosensory receptors of the invention, fluorescence-labeled tastants or auto-fluorescent tastants may be used.

Fluorescence polarization (P) is defined as:

Polarization (P)=(Iᵥ−Iₕ)/(Iᵥ+Iₕ)

where Iᵥ is the intensity of the emission light parallel to the excitation light plane and Iₕ is the intensity of the emission light perpendicular to the excitation light plane. P, being a ratio of light intensities, is a dimensionless number. For example, the Beacon and Beacon 2000 System may be used in connection with these assays. Such systems typically express polarization in millipolarization units (1 Polarization Unit=1000 mP Units).

The relationship between molecular rotation and size is described by the Perrin equation and the reader is referred to Jolley, M. E. (1991) in Journal of Analytical Toxicology, pp. 236-240, which gives a thorough explanation of this equation. Summarily, the Perrin equation states that polarization is directly proportional to the rotational relaxation time, the time that it takes a molecule to rotate through an angle of approximately 68.5 degrees. Rotational relaxation time is related to viscosity (eta.), absolute temperature (T), molecular volume (V), and the gas constant (R) by the following equation where r₀ is the maximum fluorescence anisotropy, t is the fluorescence lifetime, and tᵣ is the rotational correlation time:

$$\frac{{r}_{0}}{r} = 1 + \frac{t}{{t}_{r}}$$

The rotational relaxation time is small (about 1 nanosecond) for small molecules (e.g. fluorescein) and large (about 100 nanoseconds) for large molecules (e.g. immunoglobulins). If viscosity and temperature are held constant, rotational relaxation time, and therefore polarization, is directly related to the molecular volume. Changes in molecular volume may be due to interactions with other molecules, dissociation, polymerization, degradation, hybridization, or conformational changes of the fluorescently labeled molecule. For example, fluorescence polarization has been used to measure enzymatic cleavage of large fluorescein labeled polymers by proteases, DNases, and RNases. It also has been used to measure equilibrium binding for protein/protein interactions, antibody/antigen binding, and protein/DNA binding.

Solid State and Soluble High Throughput Assays

In yet another embodiment, the invention provides soluble assays using a hetero-oligomeric GPR113 polypeptide complex; or a cell or tissue co-expressing GPR113 polypeptides. Preferably, the cell will comprise a cell line that stably co-expresses a functional GPR113 taste receptor. In another embodiment, the invention provides solid phase based in vitro assays in a high throughput format, where the GPR113 polypeptides, or cell or tissue expressing the GPR113 polypeptides is attached to a solid phase substrate or a taste stimulating compound and contacted with a GPR113 receptor, and binding detected using an appropriate tag or antibody raised against the GPR113 receptor.

In the high throughput assays of the invention, it is possible to screen up to several thousand different compounds in a single day. In particular, each well of a microtiter plate can be used to run a separate assay against a selected potential GPR113 binding agent, activator, blocker, agonist, antagonist, or other modulator of GPR113, or, if concentration or incubation time effects are to be observed, every 5-10 wells can test a single modulator. Thus, a single standard microtiter plate can assay about 100 (e.g., 96) modulators. If 1536 well plates are used, then a single plate can easily assay from about 1000 to about 1500 different compounds. It is also possible to assay multiple compounds in each plate well. It is possible to assay several different plates per day; assay screens for up to about 6,000-20,000 different compounds are possible using the integrated systems of the invention. More recently, microfluidic approaches to reagent manipulation have been developed.

The molecule of interest can be bound to the solid state component, directly or indirectly, via covalent or non-covalent linkage, e.g., via a tag. The tag can be any of a variety of components. In general, a molecule which binds the tag (a tag binder) is fixed to a solid support, and the tagged molecule of interest (e.g., the taste transduction molecule of interest) is attached to the solid support by interaction of the tag and the tag binder.

A number of tags and tag binders can be used, based upon known molecular interactions well described in the literature. For example, where a tag has a natural binder, for example, biotin, protein A, or protein G, it can be used in conjunction with appropriate tag binders (avidin, streptavidin, neutravidin, the Fc region of an immunoglobulin, etc.). Antibodies to molecules with natural binders such as biotin are also widely available and appropriate tag binders (see, SIGMA Immunochemicals 1998 catalogue SIGMA, St. Louis Mo.).

Similarly, any haptenic or antigenic compound can be used in combination with an appropriate antibody to form a tag/tag binder pair. Thousands of specific antibodies are commercially available and many additional antibodies are described in the literature. For example, in one common configuration, the tag is a first antibody and the tag binder is a second antibody which recognizes the first antibody. In addition to antibody-antigen interactions, receptor-ligand interactions are also appropriate as tag and tag-binder pairs. For example, agonists and antagonists of cell membrane receptors (e.g., cell receptor-ligand interactions such as transferrin, c-kit, viral receptor ligands, cytokine receptors, chemokine receptors, interleukin receptors, immunoglobulin receptors and antibodies, the cadherin family, the integrin family, the selectin family, and the like; see, e.g., Pigott & Power, The Adhesion Molecule Facts Book I (1993)). Similarly, toxins and venoms, viral epitopes, hormones (e.g., opiates, steroids, etc.), intracellular receptors (e.g., which mediate the effects of various small ligands, including steroids, thyroid hormone, retinoids and vitamin D; peptides), drugs, lectins, sugars, nucleic acids (both linear and cyclic polymer configurations), oligosaccharides, proteins, phospholipids and antibodies can all interact with various cell receptors.

Synthetic polymers, such as polyurethanes, polyesters, polycarbonates, polyureas, polyamides, polyethyleneimines, polyarylene sulfides, polysiloxanes, polyimides, and polyacetates can also form an appropriate tag or tag binder. Many other tag/tag binder pairs are also useful in assay systems described herein, as would be apparent to one of skill upon review of this disclosure.

Common linkers such as peptides, polyethers, and the like can also serve as tags, and include polypeptide sequences, such as poly Gly sequences of between about 5 and 200 amino acids. Such flexible linkers are known to persons of skill in the art. For example, poly(ethylene glycol) linkers are available from Shearwater Polymers, Inc. Huntsville, Ala. These linkers optionally have amide linkages, sulfhydryl linkages, or heterofunctional linkages.

Tag binders are fixed to solid substrates using any of a variety of methods currently available. Solid substrates are commonly derivatized or functionalized by exposing all or a portion of the substrate to a chemical reagent which fixes a chemical group to the surface which is reactive with a portion of the tag binder. For example, groups which are suitable for attachment to a longer chain portion would include amines, hydroxyl, thiol, and carboxyl groups. Aminoalkylsilanes and hydroxyalkylsilanes can be used to functionalize a variety of surfaces, such as glass surfaces. The constitutive of such solid phase biopolymer arrays is well described in the literature. See, e.g., Merrifield, J. Am. Chem. Soc., 85:2149-2154 (1963) (describing solid phase synthesis of, e.g., peptides); Geysen et al., J. Immun. Meth., 102:259-274 (1987) (describing synthesis of solid phase components on pins); Frank & Doring, Tetrahedron, 44:60316040 (1988) (describing synthesis of various peptide sequences on cellulose disks); Fodor et al., Science, 251:767-777 (1991); Sheldon et al., Clinical Chemistry, 39(4):718-719 (1993); and Kozal et al., Nature Medicine, 2(7):753759 (1996) (all describing arrays of biopolymers fixed to solid substrates). Non-chemical approaches for fixing tag binders to substrates include other common methods, such as heat, cross-linking by UV radiation, and the like.

### Cell-Based Assays

In a preferred embodiment of treatment, GPR113 polypeptides are transiently or stably expressed in a eukaryotic cell either in unmodified forms or as chimeric, variant or truncated receptors with or preferably without a heterologous, chaperone sequence that facilitates its maturation and targeting through the secretory pathway. Such GPR113 polypeptides can be expressed in any eukaryotic cell, such as CHO, COS, STO, and BHK cells. Preferably, the cells comprise a functional G protein, e.g., a Gi protein, a Gs protein, a Gq protein, a Go protein, Ga15, Ga16, transducin, gustducin, or a chimeric G protein containing portions of any of the foregoing G proteins previously identified, or another G protein that is capable of coupling the chimeric receptor to an intracellular signaling pathway or to a signaling protein such as phospholipase C. Also, preferably a cell will be produced that stably expresses GPR113. The cells may comprise a heterologous protein(s) that act with GPR113 as a multimer or as a regulator thereof such as T1R3 or TRPM5. Activation of GPR113 receptors in such cells can be detected using any standard method, such as by detecting changes in intracellular calcium by detecting Fluo-4 dependent fluorescence in the cell or any of the other GPCR functional assays disclosed in this application. The results of such assays provide the basis of the experimental findings presented in this application.

Activated GPCR receptors often are substrates for kinases that phosphorylate the C-terminal tail of the receptor (and possibly other sites as well). Thus, activators will promote the transfer of ³²P from radiolabeled ATP to the receptor, which can be assayed with a scintillation counter. The phosphorylation of the C-terminal tail will promote the binding of arrestin-like proteins and will interfere with the binding of G proteins. For a general review of GPCR signal transduction and methods of assaying signal transduction, see, e.g., Methods in Enzymology, vols. 237 and 238 (1994) and volume 96 (1983); Bourne et al., Nature, 10:349:117-27 (1991); Bourne et al., Nature, 348:125-32 (1990); Pitcher et al., Annu. Rev. Biochem., 67:653-92 (1998).

GPR113 modulation may be assayed by comparing the response of GPR113 polypeptides treated with a putative GPR113 modulator to the response of an untreated control sample or a sample containing a known “positive” control. Such putative GPR113 modulators can include molecules that either inhibit or activate GPR113 polypeptide activity. In one embodiment, control samples (untreated with activators or inhibitors) are assigned a relative GPR113 activity value of 100. Inhibition of a GPR113 polypeptide is achieved when the GPR113 activity value relative to the control is about 90%, optionally 50%, optionally 25-0%. Activation of a GPR113 polypeptide is achieved e.g., when the GPR113 activity value relative to the control is increased e.g., 110%, optionally 150%, 200-500%, or 1000-2000%.

Changes in ion flux may be assessed by determining changes in ionic polarization (i.e., electrical potential) of the cell or membrane expressing a GPR113 polypeptide. One means to determine changes in cellular polarization is by measuring changes in current (thereby measuring changes in polarization) with voltage-clamp and patch-clamp techniques (see, e.g., the “cell-attached” mode, the “inside-out” mode, and the “whole cell” mode, e.g., Ackerman et al., New Engl. J Med., 336:1575-1595 (1997)). Whole cell currents are conveniently determined using the standard. Other known assays include: radiolabeled ion flux assays and fluorescence assays using voltage-sensitive dyes (see, e.g., Vestergarrd-Bogind et al., J. Membrane Biol., 88:67-75 (1988); Gonzales & Tsien, Chem. Biol., 4:269-277 (1997); Daniel et al., J. Pharmacol. Meth., 25:185-193 (1991); Holevinsky et al., J. Membrane Biology, 137:59-70 (1994)).

The effects of the test compounds upon the function of the polypeptides can be measured by examining any of the parameters described above. Any suitable physiological change that affects GPCR activity can be used to assess the influence of a test compound on the polypeptides of this invention. When the functional consequences are determined using intact cells or animals, one can also measure a variety of effects such as transmitter release, hormone release, transcriptional changes to both known and uncharacterized genetic markers (e.g., northern blots), changes in cell metabolism such as cell growth or pH changes, and changes in intracellular second messengers such as Ca²⁺, IP3, cGMP, or cAMP.

Preferred assays for GPCRs include cells that are loaded with ion or voltage sensitive dyes to report receptor activity. Assays for determining activity of such receptors can also use known agonists and antagonists for other G protein-coupled receptors as controls to assess activity of tested compounds. In assays for identifying modulatory compounds (e.g., agonists, antagonists), changes in the level of ions in the cytoplasm or membrane voltage will be monitored using an ion sensitive or membrane voltage fluorescent indicator, respectively. Among the ion-sensitive indicators and voltage probes that may be employed are those disclosed in the Molecular Probes 1997 Catalog. For G protein-coupled receptors, promiscuous G proteins such as Ga15 and Ga16 can be used in the assay of choice (Wilkie et al., Proc. Nat'l Acad. Sci., 88:10049-10053 (1991)).

Receptor activation initiates subsequent intracellular events, e.g., increases in second messengers. Activation of some G protein-coupled receptors stimulates the formation of inositol triphosphate (IP3) through phospholipase C-mediated hydrolysis of phosphatidylinositol (Berridge & Irvine, Nature, 312:315-21 (1984)). IP3 in turn stimulates the release of intracellular calcium ion stores. Thus, a change in cytoplasmic calcium ion levels, or a change in second messenger levels such as IP3 can be used to assess G protein-coupled receptor function. Cells expressing such G protein-coupled receptors may exhibit increased cytoplasmic calcium levels as a result of contribution from both calcium release from intracellular stores and extracellular calcium entry via plasma membrane ion channels.

In another embodiment, GPR113 polypeptide activity is measured by stably or transiently expressing GPR113 gene, preferably stably, in a heterologous cell with a promiscuous G protein that links the receptor to a phospholipase C signal transduction pathway (see Offermanns & Simon, J. Biol. Chem., 270:15175-15180 (1995)). In one specific embodiment, the cell line one which does not normally express GPR113 and the promiscuous G protein is Ga15 (Offermanns & Simon, supra). In another embodiment the cell is one that endogenously expresses GPR113. Modulation of taste transduction is assayed by measuring changes in intracellular Ca²⁺ levels, or IP3 levels or metabolites thereof which change in response to modulation of the GPR113 signal transduction pathway via administration of a molecule that associates with GPR113 polypeptides. Changes in Ca²⁺ levels are optionally measured using fluorescent Ca²⁺ indicator dyes and fluorometric imaging.

In another embodiment, phosphatidyl inositol (PI) hydrolysis can be analyzed according to U.S. Pat. No. 5,436,128, herein incorporated by reference. Briefly, the assay involves labeling of cells with ³H-myoinositol for 48 or more hrs. The labeled cells are treated with a test compound for one hour. The treated cells are lysed and extracted in chloroform-methanol-water after which the inositol phosphates were separated by ion exchange chromatography and quantified by scintillation counting. Fold stimulation is determined by calculating the ratio of cpm in the presence of agonist, to cpm in the presence of buffer control. Likewise, fold inhibition is determined by calculating the ratio of cpm in the presence of antagonist, to cpm in the presence of buffer control (which may or may not contain an agonist).

Other receptor assays can involve determining the level of intracellular cyclic nucleotides, e.g., cAMP or cGMP. In cases where activation of the receptor results in a decrease in cyclic nucleotide levels, it may be preferable to expose the cells to agents that increase intracellular cyclic nucleotide levels, e.g., forskolin, prior to adding a receptor-activating compound to the cells in the assay. In one embodiment, the changes in intracellular cAMP or cGMP can be measured using immunoassays. The method described in Offermanns & Simon, J. Biol. Chem., 270:15175-15180 (1995), may be used to determine the level of cAMP. Also, the method described in Felley-Bosco et al., Am. J. Resp. Cell and Mol. Biol., 11:159-164 (1994), may be used to determine the level of cGMP. Further, an assay kit for measuring cAMP and/or cGMP is described in U.S. Pat. No. 4,115,538, herein incorporated by reference.

In another embodiment, transcription levels can be measured to assess the effects of a test compound on signal transduction. A host cell containing GPR113 polypeptides of interest is contacted with a test compound for a sufficient time to effect any interactions, and then the level of gene expression is measured. The amount of time to effect such interactions may be empirically determined, such as by running a time course and measuring the level of transcription as a function of time. The amount of transcription may be measured by using any method known to those of skill in the art to be suitable. For example, mRNA expression of the protein of interest may be detected using northern blots or their polypeptide products may be identified using immunoassays. Alternatively, transcription based assays using reporter gene may be used as described in U.S. Pat. No. 5,436,128, herein incorporated by reference. The reporter genes can be, e.g., chloramphenicol acetyltransferase, luciferase, β-galactosidase β-lactamase and alkaline phosphatase. Furthermore, the protein of interest can be used as an indirect reporter via attachment to a second reporter such as green fluorescent protein (see, e.g., Mistili & Spector, Nature Biotechnology, 15:961-964 (1997)).

The amount of transcription is then compared to the amount of transcription in either the same cell in the absence of the test compound, or it may be compared with the amount of transcription in a substantially identical cell that lacks the GPR113 polypeptide(s) of interest. A substantially identical cell may be derived from the same cells from which the recombinant cell was prepared but which had not been modified by introduction of heterologous DNA. Any difference in the amount of transcription indicates that the test compound has in some manner altered the activity of the GPR113 polypeptides of interest.

Modulation of a putative taste cell specific protein can be assessed using a variety of in vitro and in vivo assays, including cell-based models as described above. Such assays can be used to test for inhibitors and activators of the protein or fragments thereof, and, consequently, inhibitors and activators thereof. Such modulators are potentially useful in medications or as flavorings to modulate fat, lipid, fatty acid or other taste modalities or taste in general or for usage as potential therapeutics for modulating a taste cell related function or phenotype involving one or several of the identified taste cell specific genes reported herein.

Assays using cells expressing the subject taste specific proteins, either recombinant or naturally occurring, can be performed using a variety of assays, in vitro, in vivo, and ex vivo, as described herein. To identify molecules capable of modulating activity thereof, assays are performed to detect the effect of various candidate modulators on activity preferably expressed in a cell.

The channel activity of ion channel proteins in particular can be assayed using a variety of assays to measure changes in ion fluxes including patch clamp techniques, measurement of whole cell currents, radiolabeled ion flux assays or a flux assay coupled to atomic absorption spectroscopy, and fluorescence assays using voltage-sensitive dyes or lithium or sodium sensitive dyes (see, e.g., Vestergarrd-Bogind et al., J. Membrane Biol. 88:67-75 (1988); Daniel et al., J. Pharmacol. Meth. 25:185-193 (1991); Hoevinsky et al., J. Membrane Biol. 137:59-70 (1994)). For example, a nucleic acid encoding a protein or homolog thereof can be injected into Xenopus oocytes or transfected into mammalian cells, preferably human cells such as COS cells. Channel activity can then be assessed by measuring changes in membrane polarization, i.e., changes in membrane potential.

A preferred means to obtain electrophysiological measurements is by measuring currents using patch clamp techniques, e.g., the “cell-attached” mode, the “inside-out” mode, and the “whole cell” mode (see, e.g., Ackerman et al., New Engl. J. Med. 336:1575-1595, 1997). Whole cell currents can be determined using standard methodology such as that described by Hamil et al., Pflugers. Archiv. 391:185 (1981).

The activity of the subject taste cell specific polypeptides can in addition to these preferred methods also be assessed using a variety of other in vitro and in vivo assays to determine functional, chemical, and physical effects, e.g., measuring the binding thereof to other molecules, including peptides, small organic molecules, and lipids; measuring protein and/or RNA levels, or measuring other aspects of the subject polypeptides, e.g., transcription levels, or physiological changes that affects the taste cell specific protein's activity. When the functional consequences are determined using intact cells or animals, one can also measure a variety of effects such as changes in cell growth or pH changes or changes in intracellular second messengers such as IP3, cGMP, or cAMP, or components or regulators of the phospholipase C signaling pathway. Such assays can be used to test for both activators and inhibitors of GPR113 proteins. Modulators thus identified are useful for, e.g., many diagnostic and therapeutic applications.

### In Vitro Assays

Assays to identify compounds with modulating activity on the subject genes are preferably performed in vitro. The assays herein preferably use full length protein according to the invention or a variant thereof. This protein can optionally be fused to a heterologous protein to form a chimera. In the assays exemplified herein, cells which express the full-length polypeptide are preferably used in high throughput assays to identify compounds that modulate gene function. Alternatively, purified recombinant or naturally occurring protein can be used in the in vitro methods of the invention. In addition to purified protein or fragments thereof, the recombinant or naturally occurring taste cell protein can be part of a cellular lysate or a cell membrane. As described below, the binding assay can be either solid state or soluble. Preferably, the protein, fragment thereof or membrane is bound to a solid support, either covalently or non-covalently. Often, the in vitro assays of the invention are ligand binding or ligand affinity assays, either non-competitive or competitive (with known extracellular ligands such as fats and lipid compounds that specifically bind or activate the subject GPR113 polypeptide. These in vitro assays include measuring changes in spectroscopic (e.g., fluorescence, absorbance, refractive index), hydrodynamic (e.g., shape), chromatographic, or solubility properties for the protein.

Preferably, a high throughput binding assay is performed in which the protein is contacted with a potential modulator and incubated for a suitable amount of time. A wide variety of modulators can be used, as described below, including small organic molecules, peptides, antibodies, and ligand analogs. A wide variety of assays can be used to identify modulator binding, including labeled protein-protein binding assays, electrophoretic mobility shifts, immunoassays, enzymatic assays such as phosphorylation assays, and the like. In some cases, the binding of the candidate modulator is determined through the use of competitive binding assays, where interference with binding of a known ligand is measured in the presence of a potential modulator. In such assays the known ligand is bound first, and then the desired compound i.e., putative enhancer is added. After the particular protein is washed, interference with binding, either of the potential modulator or of the known ligand, is determined. Often, either the potential modulator or the known ligand is labeled.

In addition, high throughput functional genomics assays can also be used to identify modulators of fat taste or fat metabolism and for the identification of compounds that disrupt protein interactions between the subject taste specific polypeptide and other proteins to which it binds. Such assays can, e.g., monitor changes in cell surface marker expression, changes in intracellular calcium, or changes in membrane currents using either cell lines or primary cells. Typically, the cells are contacted with a cDNA or a random peptide library (encoded by nucleic acids). The cDNA library can comprise sense, antisense, full length, and truncated cDNAs. The peptide library is encoded by nucleic acids. The effect of the cDNA or peptide library on the phenotype of the cells is then monitored, using an assay as described above. The effect of the cDNA or peptide can be validated and distinguished from somatic mutations, using, e.g., regulatable expression of the nucleic acid such as expression from a tetracycline promoter. cDNAs and nucleic acids encoding peptides can be rescued using techniques known to those of skill in the art, e.g., using a sequence tag.

Proteins interacting with the protein encoded by a cDNA according to the invention can be isolated using a yeast two-hybrid system, mammalian two hybrid system, or phage display screen, etc. Targets so identified can be further used as bait in these assays to identify additional components that may interact with the particular ion channel, receptor or transporter protein which members are also targets for drug development (see, e.g., Fields et al., Nature 340:245 (1989); Vasavada et al., Proc. Nat'l Acad. Sci. USA 88:10686 (1991); Fearon et al., Proc. Nat'l Acad. Sci. USA 89:7958 (1992); Dang et al., Mol. Cell. Biol. 11:955 (1991); Chien et al., Proc. Nat'l Acad. Sci. USA 9578 (1991); and U.S. Pat. Nos. 5,283,173, 5,667,973, 5,468,6 15, 5,525,490, and 5,637,463).

### Cell-Based In Vitro Assays

In preferred embodiments, wild-type and mutant GPR113 proteins are expressed in a cell, and functional, e.g., physical and chemical or phenotypic, changes are assayed to identify modulators that modulate function or which restore the function of mutant genes, e.g., those having impaired gating function. Cells expressing proteins can also be used in binding assays. Any suitable functional effect can be measured, as described herein. For example, changes in membrane potential, changes in intracellular electrolyte levels, and ligand binding are all suitable assays to identify potential modulators using a cell based system. Suitable cells for such cell based assays include both primary cells and recombinant cell lines engineered to express a protein. The subject taste cell specific proteins therefore can be naturally occurring or recombinant. Also, as described above, fragments of these proteins or chimeras with ion channel activity can be used in cell based assays. For example, a transmembrane domain of an ion channel or GPCR or transporter gene according to the invention can be fused to a cytoplasmic domain of a heterologous protein, preferably a heterologous ion channel protein. Such a chimeric protein would have ion channel activity and could be used in cell based assays of the invention. In another embodiment, a domain of the taste cell specific protein, such as the extracellular or cytoplasmic domain, is used in the cell-based assays of the invention.

In another embodiment, cellular polypeptide levels of the particular target taste polypeptide can be determined by measuring the level of protein or mRNA. The level of protein or proteins related to ion channel activation are measured using immunoassays such as western blotting, ELISA and the like with an antibody that selectively binds to the polypeptide or a fragment thereof. For measurement of mRNA, amplification, e.g., using PCR, LCR, or hybridization assays, e.g., northern hybridization, RNAse protection, dot blotting, are preferred. The level of protein or mRNA is detected using directly or indirectly labeled detection agents, e.g., fluorescently or radioactively labeled nucleic acids, radioactively or enzymatically labeled antibodies, and the like, as described herein.

Alternatively, protein expression can be measured using a reporter gene system. Such a system can be devised using a promoter of the target gene operably linked to a reporter gene such as chloramphenicol acetyltransferase, firefly luciferase, bacterial luciferase, β-galactosidase and alkaline phosphatase. Furthermore, the protein of interest can be used as an indirect reporter via attachment to a second reporter such as red or green fluorescent protein (see, e.g., Mistili & Spector, Nature Biotechnology 15:961-964 (1997)). The reporter construct is typically transfected into a cell. After treatment with a potential modulator, the amount of reporter gene transcription, translation, or activity is measured according to standard techniques known to those of skill in the art.

In another embodiment, a functional effect related to signal transduction can be measured. An activated or inhibited ion channel or GPCR or transporter will potentially alter the properties of target enzymes, second messengers, channels, and other effector proteins. The examples include the activation of phospholipase C and other signaling systems. Downstream consequences can also be examined such as generation of diacyl glycerol and IP3 by phospholipase C.

### Animal Models

Animal models also find potential use in screening for modulators of gene activity. Transgenic animal technology results in gene overexpression, whereas siRNA and gene knockout technology results in absent or reduced gene expression following homologous recombination with an appropriate gene targeting vector. The same technology can also be applied to make knockout cells. When desired, tissue-specific expression or knockout of the target gene may be necessary. Transgenic animals generated by such methods find use as animal models of responses related to the gene target. For example such animals expressing a gene or genes according to the invention may be used to derive supertaster phenotypes such as for use in screening of chemical and biological toxins, rancid/spoiled/contaminated foods, and beverages or for screening for therapeutic compounds that modulate taste stem cell differentiation.

Knockout cells and transgenic mice can be made by insertion of a marker gene or other heterologous gene into an endogenous gene site in the mouse genome via homologous recombination. Such mice can also be made by substituting an endogenous gene with a mutated version of the target gene, or by mutating an endogenous gene, e.g., by exposure to known mutagens.

A DNA construct is introduced into the nuclei of embryonic stem cells. Cells containing the newly engineered genetic lesion are injected into a host mouse embryo, which is re-implanted into a recipient female. Some of these embryos develop into chimeric mice that possess germ cells partially derived from the mutant cell line. Therefore, by breeding the chimeric mice it is possible to obtain a new line of mice containing the introduced genetic lesion (see, e.g., Capecchi et al., Science 244:1288 (1989)). Chimeric targeted mice can be derived according to Hogan et al., Manipulating the Mouse Embryo: A Laboratory Manual (1988) and Teratocarcinomas and Embryonic Stem Cells: A Practical Approach (Robertson, ed., 1987).

### Candidate Modulators

The compounds tested as modulators of the putative taste-related proteins or other non-taste related functions and phenotypes involving taste cells can be any small organic molecule, or a biological entity, such as a protein, e.g., an antibody or peptide, a sugar, a nucleic acid, e.g., an antisense oligonucleotide or a ribozyme, or a lipid. Alternatively, modulators can be genetically altered versions of a protein. Typically, test compounds will be small organic molecules, peptides, lipids, and lipid analogs. In one embodiment, the compound is a fat, lipid, fatty acid, or oil, either naturally occurring or synthetic.

Essentially any chemical compound can be used as a potential modulator or ligand in the assays of the invention, although most often compounds that can be dissolved in aqueous or organic (especially DMSO-based) solutions are used. The assays are designed to screen large chemical libraries by automating the assay steps and providing compounds from any convenient source to assays, which are typically run in parallel (e.g., in microtiter formats on microtiter plates in robotic assays). It will be appreciated that there are many suppliers of chemical compounds, including Sigma (St. Louis, Mo.), Aldrich (St. Louis, Mo.), Sigma-Aldrich (St. Louis, Mo.), Fluka Chemika-Biochemica Analytika (Buchs Switzerland) and the like.

In one preferred embodiment, high throughput screening methods involve providing a combinatorial small organic molecule or peptide library containing a large number of potential therapeutic compounds (potential modulator or ligand compounds). Such “combinatorial chemical libraries” or “ligand libraries” are then screened in one or more assays, as described herein, to identify those library members (particular chemical species or subclasses) that display a desired characteristic activity. The compounds thus identified can serve as conventional “lead compounds” or can themselves be used as potential or actual therapeutics.

A combinatorial chemical library is a collection of diverse chemical compounds generated by either chemical synthesis or biological synthesis, by combining a number of chemical “building blocks” such as reagents. For example, a linear combinatorial chemical library such as a polypeptide library is formed by combining a set of chemical building blocks (amino acids) in every possible way for a given compound length (i.e., the number of amino acids in a polypeptide compound). Millions of chemical compounds can be synthesized through such combinatorial mixing of chemical building blocks.

Preparation and screening of combinatorial chemical libraries is well known to those of skill in the art. Such combinatorial chemical libraries include, but are not limited to, peptide libraries (see, e.g., U.S. Pat. No. 5,010,175, Furka, Int. J. Pept. Prot. Res. 37:487-493 (1991) and Houghton et al., Nature 355:84-88 (1991)). Other chemistries for generating chemical diversity libraries can also be used. Such chemistries include, but are not limited to: peptoids (e.g., PCT Publication No. WO 91/19735), encoded peptides (e.g., PCT Publication No. WO 93/20242), random bio-oligomers (e.g., PCT Publication No. WO 92/00091), benzodiazepines (e.g., U.S. Pat. No. 5,288,515), diversomers such as hydantoins, benzodiazepines and dipeptides (Hobbs et al., Proc. Nat. Acad. Sci. USA 90:6909-6913 (1993)), vinylogous polypeptides (Hagihara et al., J. Amer. Chem. Soc. 1 15:6568 (1992)), nonpeptidal peptidomimetics with glucose scaffolding (Hirschmann et al., J. Amer. Chem. Soc. 1 15:9217-9218 (1992)), analogous organic syntheses of small compound libraries (Chen et al., J. Amer. Chem. Soc. 116:2661 (1994)), oligocarbamates (Cho et al., Science 261:1303 (1993)), and/or peptidyl phosphonates (Campbell et al., J. Org. Chem. 59:658 (1994)), nucleic acid libraries (see Ausubel, Berger and Sambrook, all supra), peptide nucleic acid libraries (see, e.g., U.S. Pat. No. 5,539,083), antibody libraries (see, e.g., Vaughn et al., Nature Biotechnology, 15(3):309-3 15 (1996) and PCT/US96/10287), carbohydrate libraries (see, e.g., Liang et al., Science, 274:1520-1522 (1996) and U.S. Pat. No. 5,593,853), small organic molecule libraries (see, e.g., benzodiazepines, Baum C&EN, January 18, page 33 (1993); isoprenoids, U.S. Pat. No. 5,569,588; thiazolidinones and metathiazanones, U.S. Pat. No. 5,559,974; pyrrolidines, U.S. Pat. Nos. 5,525,735 and 5,519,134; morpholino compounds, U.S. Pat. No. 5,506,337; benzodiazepines, U.S. Pat. No. 5,288,515, and the like).

Devices for the preparation of combinatorial libraries are commercially available (see, e.g., 357 MPS, 390 MPS, Advanced Chem Tech, Louisville Ky., Symphony, Rainin, Woburn, Mass., 433A Applied Biosystems, Foster City, Calif., 9050 Plus, Millipore, Bedford, Mass.). In addition, numerous combinatorial libraries are themselves commercially available (see, e.g., ComGenex, Princeton, N.J., Asinex, Moscow, Ru, Tripos, Inc., St. Louis, Mo., ChemStar, Ltd, Moscow, RU, 3D Pharmaceuticals, Exton, Pa., Martek Biosciences, Columbia, Md.). C. Solid State and Soluble High Throughput Assays

Additionally soluble assays can be affected using a target taste specific protein, or a cell or tissue expressing a target taste protein disclosed herein, either naturally occurring or recombinant. Still alternatively, solid phase based in vitro assays in a high throughput format can be effected, where the protein or fragment thereof, such as the cytoplasmic domain, is attached to a solid phase substrate. Any one of the assays described herein can be adapted for high throughput screening, e.g., ligand binding, calcium flux, change in membrane potential, etc.

In the high throughput assays of the invention, either soluble or solid state, it is possible to screen several thousand different modulators or ligands in a single day. This methodology can be used for assaying proteins in vitro, or for cell-based or membrane-based assays comprising a protein. In particular, each well of a microtiter plate can be used to run a separate assay against a selected potential modulator, or, if concentration or incubation time effects are to be observed, every 5-10 wells can test a single modulator. Thus, a single standard microtiter plate can assay about 100 (e.g., 96) modulators. If 1536 well plates are used, then a single plate can easily assay from about 100-about 1500 different compounds. It is possible to assay many plates per day; assay screens for up to about 6,000, 20,000, 50,000, or more than 100,000 different compounds are possible using the integrated systems of the invention.

For a solid state reaction, the protein of interest or a fragment thereof, e.g., an extracellular domain, or a cell or membrane comprising the protein of interest or a fragment thereof as part of a fusion protein can be bound to the solid state component, directly or indirectly, via covalent or non-covalent linkage e.g., via a tag. The tag can be any of a variety of components. In general, a molecule which binds the tag (a tag binder) is fixed to a solid support, and the tagged molecule of interest is attached to the solid support by interaction of the tag and the tag binder.

A number of tags and tag binders can be used, based upon known molecular interactions well described in the literature. For example, where a tag has a natural binder, for example, biotin, protein A, or protein G, it can be used in conjunction with appropriate tag binders (avidin, streptavidin, neutravidin, the Fc region of an immunoglobulin, etc.) Antibodies to molecules with natural binders such as biotin are also widely available and appropriate tag binders; see, SIGMA Immunochemicals 1998 catalogue SIGMA, St. Louis Mo.).

Similarly, any haptenic or antigenic compound can be used in combination with an appropriate antibody to form a tag/tag binder pair. Thousands of specific antibodies are commercially available and many additional antibodies are described in the literature. For example, in one common configuration, the tag is a first antibody and the tag binder is a second antibody which recognizes the first antibody. In addition to antibody-antigen interactions, receptor-ligand interactions are also appropriate as tag and tag-binder pairs. For example, agonists and antagonists of cell membrane receptors (e.g., cell receptor-ligand interactions such as transferrin, c-kit, viral receptor ligands, cytokine receptors, chemokine receptors, interleukin receptors, immunoglobulin receptors and antibodies, the cadherin family, the integrin family, the selectin family, and the like; see, e.g., Pigott & Power, The Adhesion Molecule Facts Book I (1993). Similarly, toxins and venoms, viral epitopes, hormones (e.g., opiates, steroids, etc.), intracellular receptors (e.g. which mediate the effects of various small ligands, including steroids, thyroid hormone, retinoids and vitamin D; peptides), drugs, lectins, sugars, nucleic acids (both linear and cyclic polymer configurations), oligosaccharides, proteins, phospholipids and antibodies can all interact with various cell receptors.

Synthetic polymers, such as polyurethanes, polyesters, polycarbonates, polyureas, polyamides, polyethyleneimines, polyarylene sulfides, polysiloxanes, polyimides, and polyacetates can also form an appropriate tag or tag binder. Many other tag/tag binder pairs are also useful in assay systems described herein, as would be apparent to one of skill upon review of this disclosure.

Common linkers such as peptides, polyethers, and the like can also serve as tags, and include polypeptide sequences, such as poly Gly sequences of between about 5 and 200 amino acids. Such flexible linkers are known to persons of skill in the art. For example, poly(ethylene glycol) linkers are available from Shearwater Polymers, Inc. Huntsville, Ala. These linkers optionally have amide linkages, sulfhydryl linkages, or heterofunctional linkages.

Tag binders are fixed to solid substrates using any of a variety of methods currently available. Solid substrates are commonly derivatized or functionalized by exposing all or a portion of the substrate to a chemical reagent which fixes a chemical group to the surface which is reactive with a portion of the tag binder. For example, groups which are suitable for attachment to a longer chain portion would include amines, hydroxyl, thiol, and carboxyl groups. Aminoalkylsilanes and hydroxyalkylsilanes can be used to functionalize a variety of surfaces, such as glass surfaces. The construction of such solid phase biopolymer arrays is well described in the literature. See, e.g., Merrifield, J. Am. Chem. Soc. 85:2 159-2155 (1963) (describing solid phase synthesis of, e.g., peptides); Geysen et al., J. Immunol. Meth. 102:259-274 (1987) (describing synthesis of solid phase components on pins); Frank & Doring, Tetrahedron 44:6031-6040 (1988) (describing synthesis of various peptide sequences on cellulose disks); Fodor et al., Science, 251:767-777 (1991); Sheldon et al., Clinical Chemistry 39(4):718-719 (1993); and Kozal et al., Nature Medicine 2(7):753-759 (1996) (all describing arrays of biopolymers fixed to solid substrates). Non-chemical approaches for fixing tag binders to substrates include other common methods, such as heat, cross-linking by UV radiation, and the like.

Having described the invention in detail supra, the examples provided infra further illustrate some preferred embodiments of the invention. These examples are provided only for purposes of illustration and should not be construed as limiting the subject invention.

### EXAMPLES

### Example 1

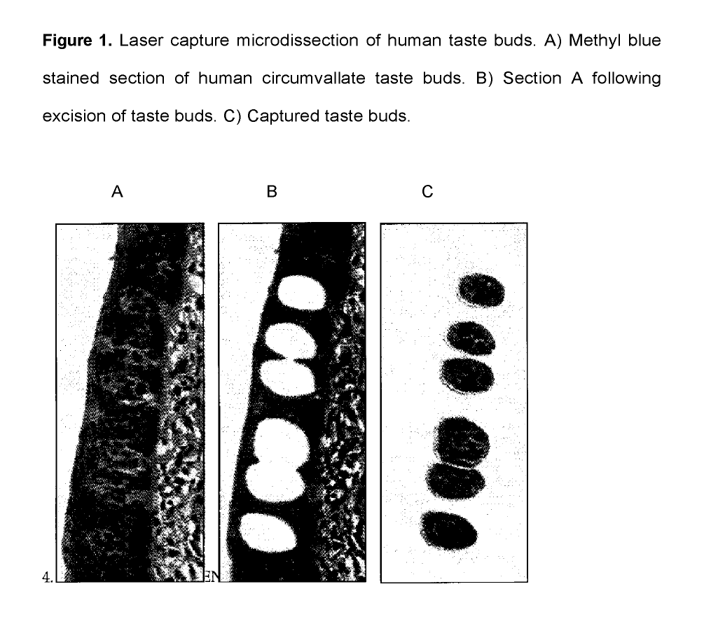

This experiment relates in part to the experiments the results of which are contained in FIG. 1 which is exemplary of the results obtained with laser capture microdissection (LCM) on human taste buds. Panel A in the figure shows methyl blue stained section of human circumvallate taste buds. Panel B shows section A after the excision of taste buds. Panel C shows the excised captured human taste buds. Human taste buds were used to identify the genes which are specifically expressed therein including the subject GPCR gene, GPR113. Particularly, the inventors identified this human taste specific gene by the use of microarray analyses and quantitative polymerase chain reaction (PCR). Using these methods the inventors demonstrated taste specific gene expression in humans (in addition to primate) and validated the specificity of expression by a quantitative method (qPCR or “TaqMan”). The genes selected for examination by the inventors including the subject GPR113 gene and others all encode multi-span transmembrane proteins, and based thereon they should all encode a polypeptide having a function that affects human taste or another human taste bud related biological function such as those mentioned herein. Because the inventors previously used microarray gene expression detection methods to assess and identify the expression of taste specific genes in primate (macaque) taste tissues, and since macaques and humans are closely evolutionarily related, genes identified as being taste specific in the primate experiments were selected to be validated in human taste buds using real time polymerase chain reaction (TaqMan qPCR).

In order to isolate human taste buds the inventors performed laser capture microdissection (LCM) as exemplified in FIG. 1. In general, selected cells or groups of cells from tissue sections were isolated based on morphological distinctions. The inventors are able to readily identify these desired taste bud structures in sections of human tongue. In this specific example tissue collection was limited to taste buds (TB) in circumvallate papillae and, as a control, cells from the adjacent lingual epithelium (LE). An example of sections used in sample collection is shown in FIG. 1. Multiple LCM preparations from each of 3 human donors were pooled (˜4500 cells per sample), RNA extracted and amplified by WT-Ovation Pico RNA Amplification System (NuGEN Technologies, Inc) and analyzed using TaqMan technology to determine specific levels of gene expression in the TB and LE pools.

The expression of human taste-specific genes was quantified by TaqMan in LCM derived cDNA from both LE and TB from the same donors. Only genes determined to be expressed only in human TB but not in LE or at much lower levels in LE were considered to be taste-specific genes. Gene expression is measured in TaqMan as a CT (cycle threshold) value. Briefly, the CT value for a given sample is determined by the PCR cycle at which the amount of gene-specific PCR product (as measured by fluorescence) reaches a set value. In other words, it represents the number of cycles needed to detect a particular gene; for highly expressed genes, the threshold will be reached early in the PCR run and the CT value will be relatively low (<35) while genes with very low or no expression will not reach the threshold before cycle 35. Expression of genes with CT values>40 are defined as not detectable. For the majority of genes identified as being taste specific, including GPR113, the expression of this gene was not detected in LE samples (CT>40) but was readily detectable in TB samples (CT<35).

### Example 2

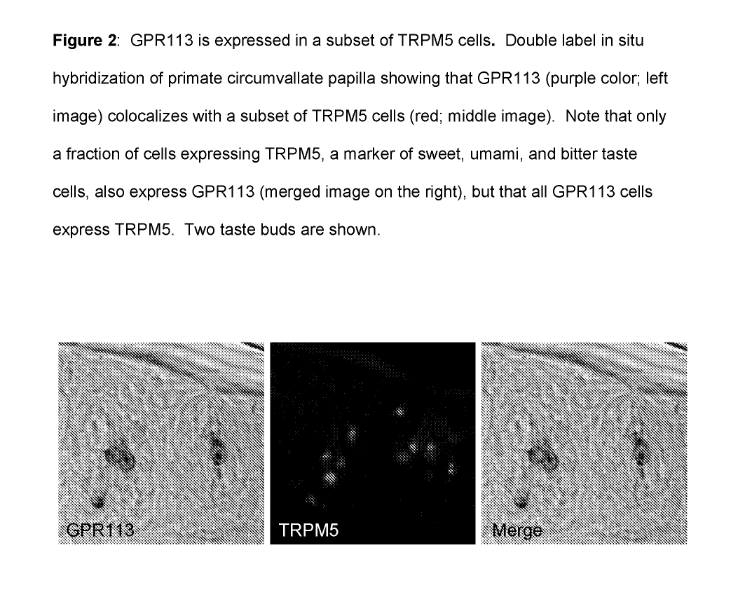

This example relates to the double label in situ hybridization experiment contained in FIG. 2. This hybridization experiment used primate circumvallate papilla and revealed that the taste cell specific gene GPR113 (purple color; left image) colocalizes with a subset of TRPM5 cells (red; middle image). It can be seen from the figure that that only a fraction of cells expressing TRPM5, a marker of sweet, umami, and bitter taste cells, also express GPR113 (merged image on the right), but that all GPR113 cells express TRPM5. Two taste buds are shown.

### Example 3

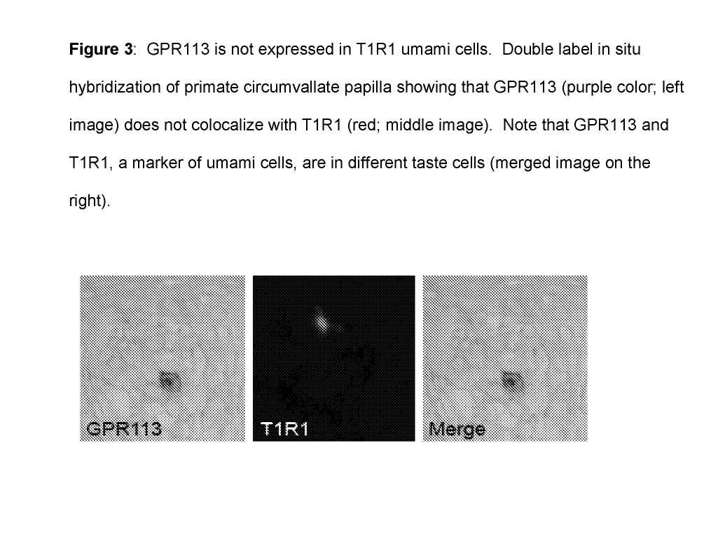

This example corresponds to the in situ hybridization experiments in FIG. 3. The results show that GPR113 is not expressed in T1R1 umami cells. Double label in situ hybridization of primate circumvallate papilla shows that GPR113 (purple color; left image) does not colocalize with T1R1 (red; middle image). Note that GPR113 and T1R1, a market of umami cells, are in different taste cells (merged image on the right) EXAMPLE 4:

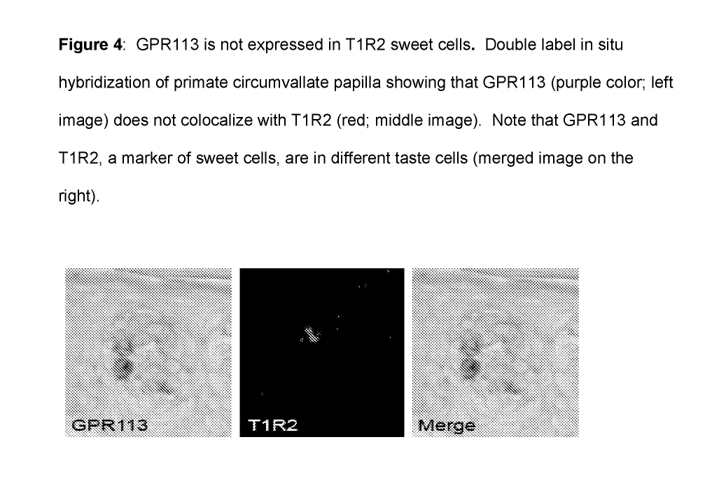

This example which corresponds to the experiment in FIG. 4 shows that GPR113 is not expressed in T1R2 sweet cells. Double label in situ hybridization of primate circumvallate papilla showing that GPR113 (purple color; left image) does not colocalize with T1R2 (red; middle image). Note that GPR113 and T1R2, a marker of sweet cells, are in different taste cells (merged image on the right).

### Example 5

This example which corresponds to the experiment in FIG. 5 shows that GPR113 is expressed in a subset of T1R3 cells. Double label in situ hybridization of primate circumvallate papilla showing that GPR113 (purple color; left image) does colocalize with a subset of T1R3 cells (red; middle image). Note that GPR113 is always expressed in cells with T1R3, but that there are T1R3 cells that do not express GPR113 (merged image on the right). These T1R3 cells that do not express GPR113 likely coexpress either T1R1 or T1R2. The T1R3 only cells are a new population of taste cells that coexpress GPR113.

### Example 6

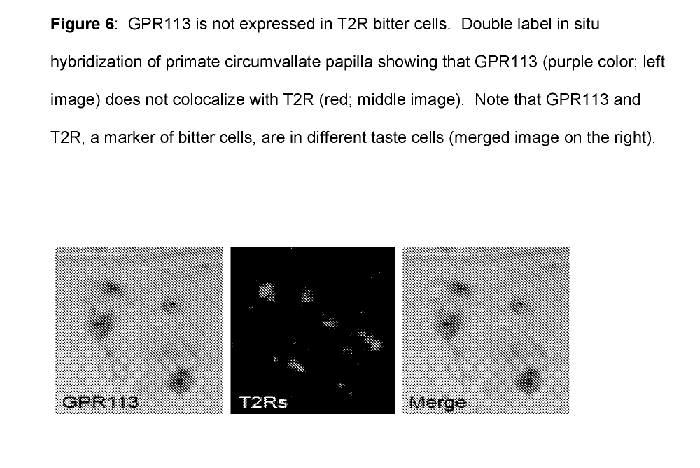

This example which corresponds to the experiment in FIG. 6 shows that GPR113 is not expressed in T2R bitter cells. Double label in situ hybridization of primate circumvallate papilla showing that GPR113 (purple color; left image) does not colocalize with T2R (red; middle image). Note that GPR113 and T2R, a marker of bitter cells, are in different taste cells (merged image on the right).

### Example 7

Using quantitative polymerase chain reaction (qPCR) we have demonstrated that GPR113 is expressed at relatively high levels in the CV taste buds of mice, primates and humans with little or no detectable expression in lingual epithelium. (See Table 1) below:

TABLE 1
qPCR expression of GPR113 in taste bud and lingual epithelium
collected by laser capture microdissection.

CT Values

| Species | Taste bud | Lingual epithelium |
|---|---|---|
| Murine | 22.83 | 40 |
| Primate | 28.50 | 40 |
| Human | 29.44 | 40 |

CT values of 40 indicate no expression.

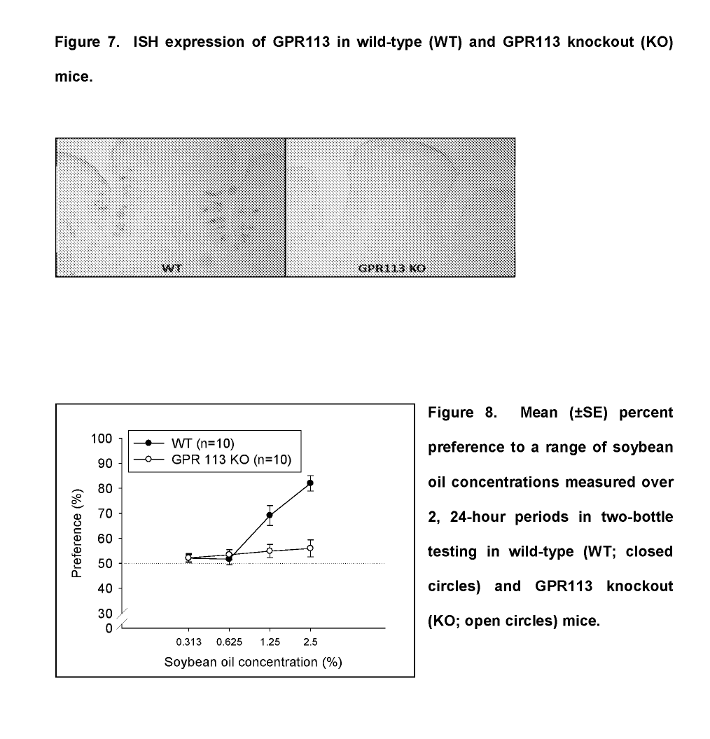

In addition, using in situ hybridization (ISH) as described above we have demonstrated that GPR113 KO mice have no visible expression of GPR113 mRNA in CV as expected (FIG. 7). As noted above, histological characterization of GPR113 in wild-type taste tissue has revealed that a subset of cells expressing GPR113 co-express T1R3 taste receptors but there is no overlap with cells expressing T1R2 or T2R05. Additionally, while GPR113 expression overlaps with TRPM5 expression in a subset of cells, as shown above there is no overlap with cell populations expressing PKD2L1 or α-gustducin. The profile of GPR113 expression therefore suggests that GPR113 represents a new taste cell type and that this receptor may regulate fat, fatty acid or lipid taste or fat, fatty acid or lipid metabolism and regulate dietary control (especially fat, fatty acid or lipid consumption) alone or in association with T1R3 and/or TRPM5.

### Example 8

### Behavioral Analysis of GPR113 KO Mice

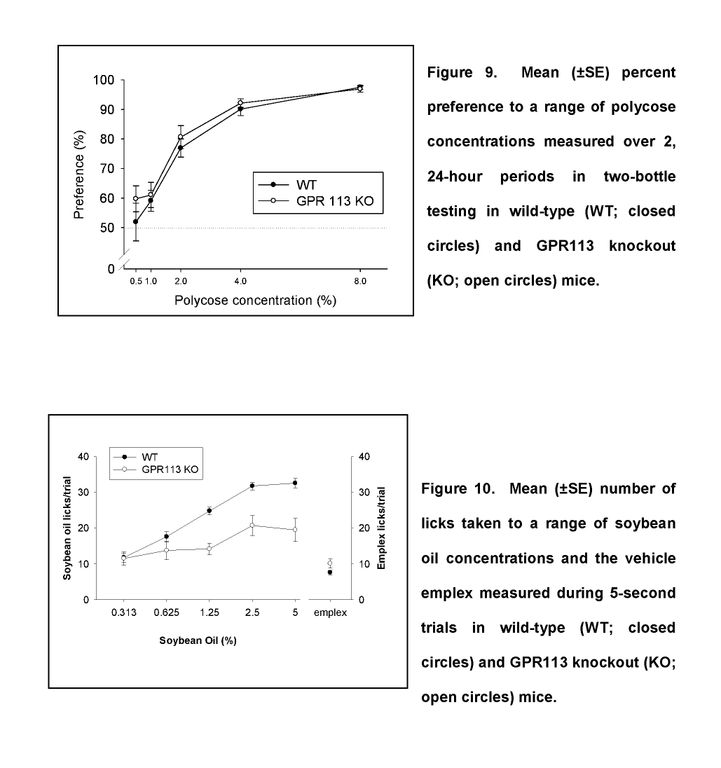

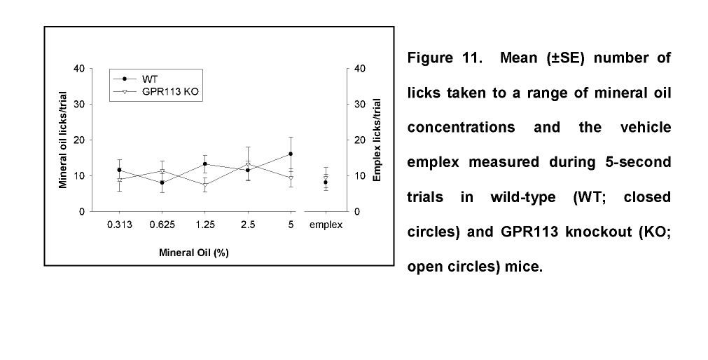

Several groups of mice underwent behavioral testing. In two-bottle intake tests, GPR113 KO mice showed decreased preferences for soybean oil (FIG. 8), the non-nutritive sefa soyate oil, and intralipid (emulsified soybean oil). Polycose preference (FIG. 9) was not different between wild-type (WT) and GPR113 KO mice suggesting that these effects are specific to the oils tested and not a general effect on caloric stimuli. We also tested groups of mice in brief-access licking paradigms. WT mice increased licking in response to increasing concentrations of soybean oil (FIG. 10), linoleic acid, oleic acid, corn oil and sefa soyate oil. This response was absent or significantly diminished in GPR113 KO animals. Licking to tastants from other modalities was not affected. Specifically, WT and GPR113 KO mice responded similarly to polycose, sucrose, NaCl, KCl, citric acid, and quinine. Mineral oil was tested as a control for viscosity. Neither WT nor KO mice increased licking with increasing concentration of this tasteless oil (FIG. 11).

### Example 9

### Glossopharyngeal Nerve Transection

Histological findings localized GPR113 expression to the CV papillae, a region of the oral cavity innervated by the glossopharyngeal nerve. Based thereon the inventors predicted that glossopharyngeal nerve transaction (GLX) in WT mice should at least partially recapitulate the deficits observed in GPR113 KO mice.

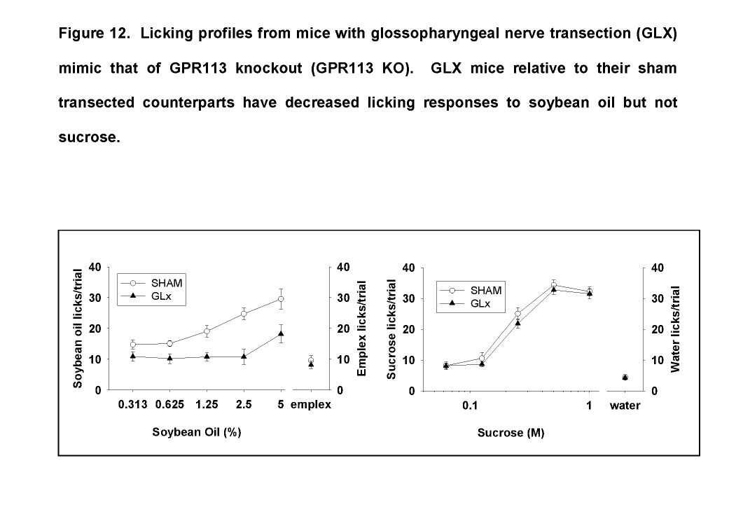

C57Bl/6 mice (Harlan) were trained to lick in the brief access licking. Following training mice were balanced for body weight and average number of licks per trial to water during training and assigned to a surgery group. Mice were allowed to recover for at least two weeks following surgery. They were given two days of licking to water (shutter training), food was taken away overnight and they were tested for their licking responses to soybean oil in emplex over 2 days of testing. The next week they were tested in the same manner to sucrose. Following the last day of testing, mice were euthanized and their tongues were taken for histological analysis. CV papilla were cross sectioned and stained with hematoxylin/eosin. An observer blind to the surgical condition counted taste buds. Mice that had greater than 3 taste buds were excluded from the statistical analysis. Concentration-dependent licking to soybean oil was clearly attenuated in GLX mice relative to SHAM operated controls. By contrast, both surgical groups displayed identical increases in licking to sucrose as concentrations were increased (FIG. 12).

### Example 10

Transient Co-Expression of GPR113 with Different G Proteins

Over-expression of most, if not all, GPCRs results in measurable constitutive activity, that is, receptor signaling in the absence of a ligand for that receptor. Based thereon, experiments were conducted using 2 assay formats in order to potentially demonstrate GPR113 constitutive activity. In these experiments, constitutive GPR113 signaling was measured using a Gq-mediated pathway and 2 different assays.

In the first assay format, experiments were conducted wherein the subject GPR113 gene was transiently co-expressed with various G proteins and basal levels of IP1 in transfected cells were measured with an HTRF-based kit from Cisbio.

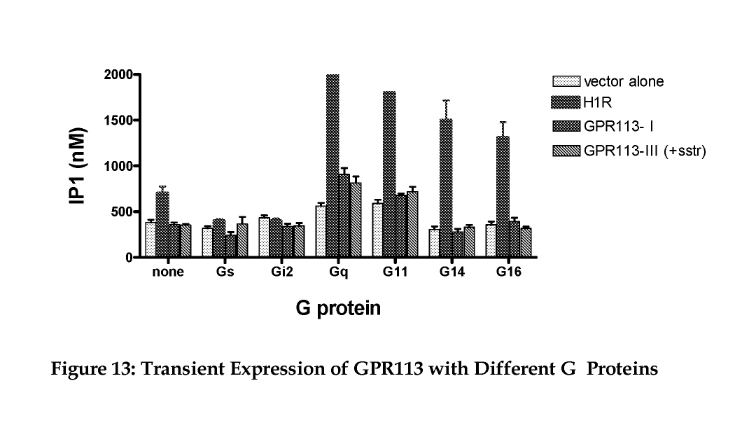

The results of these experiments are in FIG. 13. As shown therein, these experiments revealed that the co-expression of GPR113 with Gq results in elevated levels of IP1 relative to control (Gq with empty vector) indicating that GPR113 can signal through a Gq-mediated pathway. The histamine receptor (H1R), a known Gq-coupled receptor, further couples to Gq as well as other members of the Gq family in this assay.

### Example 11

Transient Co-Expression of GPR113 with Different Amounts of Gq Proteins

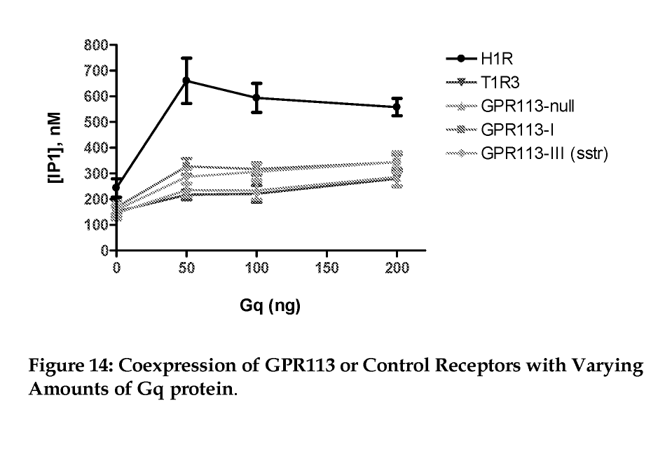

As shown in FIG. 14, experiments were also conducted wherein the subject GPR113 gene or control receptors were transiently co-expressed with varying amounts of Gq and IP1 levels measured with the same Cisbio kit. The results of these experiments are contained in FIG. 14. It can be seen from these results that the GPR113 isoforms I and III consistently generated higher IP1 levels than the negative controls, (T1R3 or a GPR113 construct containing a frame-shift mutation) (GPR113-null).

### Example 12

Transient Co-Expression of GPR113 with Different Amounts of GSQ Chimeric Proteins

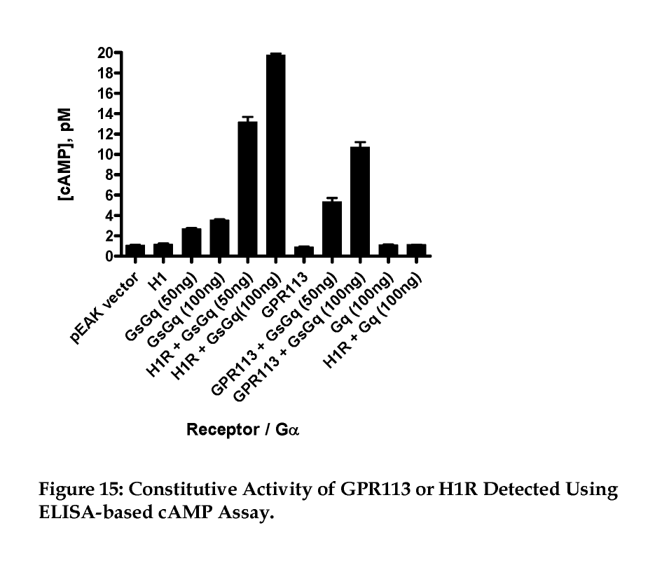

As shown in FIG. 15, experiments were also conducted wherein constitutive activity was measured in the 2^{nd} assay format. In these experiments an ELISA-based cAMP assay (Perkin Elmer) was effected in which GPR113 or H1R was co-expressed with the same G protein chimera, Gsq5. This G protein chimera consists of a Gs subunit which contains a substitution of the last 5 amino acids with those of Gq. The Gsq5 chimera provides the Gs domain required for stimulation of cAMP levels and the last 5 amino acids provide for coupling by Gq-coupled receptors.

H1R and GPR113 constitutive activity is detected when the receptor is co-expressed with 2 different amounts of the Gsq5 chimera compared to Gsq5 alone. The results of these experiments are contained in FIG. 15. It can be seen therefrom that no activity was detected when GPR113 is expressed alone.

### Example 13

Co-Expression of GPR113 with Different Amounts of Gq Proteins

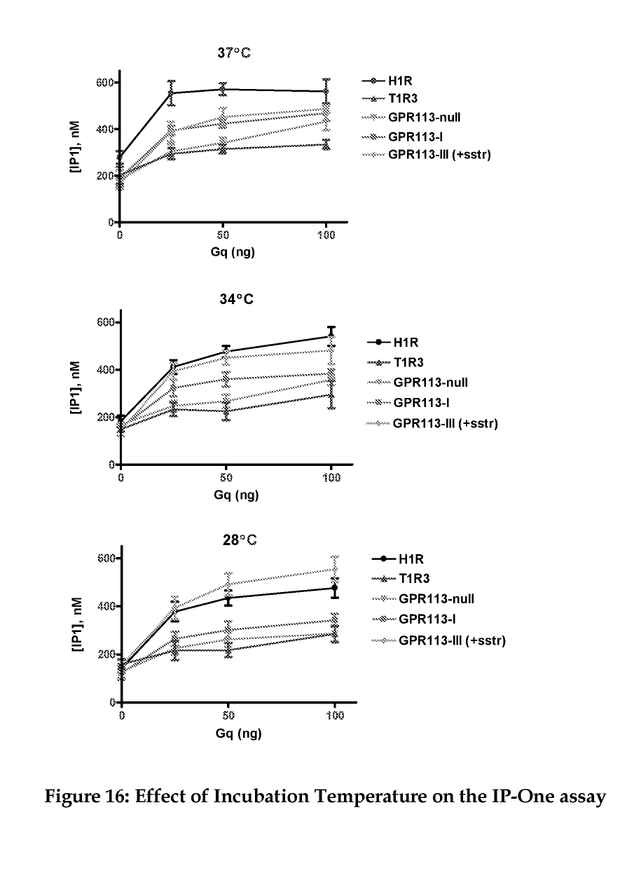

As shown in FIG. 16, additional experiments were conducted wherein GPR113 or control receptors were co-expressed with varying amounts of Gq and IP1 levels measured with the Cisbio kit. Cells were incubated at 37 C for the first 24 hours after transfection followed by transfer of some cells to 34 C or 28 C for an additional 24 hrs before performing the assay. The results of these experiments revealed that the response of cells expressing the GPR113-isoform III containing an sstr tag comprised of the N-terminal amino acids of the somatostatin 3 receptor (which tag facilitates the targeting of GPR113 to the cell surface) increased relative to the negative controls with decreasing incubation temperature. The result is a larger assay window.

### Example 14

Co-Expression of GPR113 or Control Receptors with Varying Amounts of the Gsq5 Chimeric G-Protein

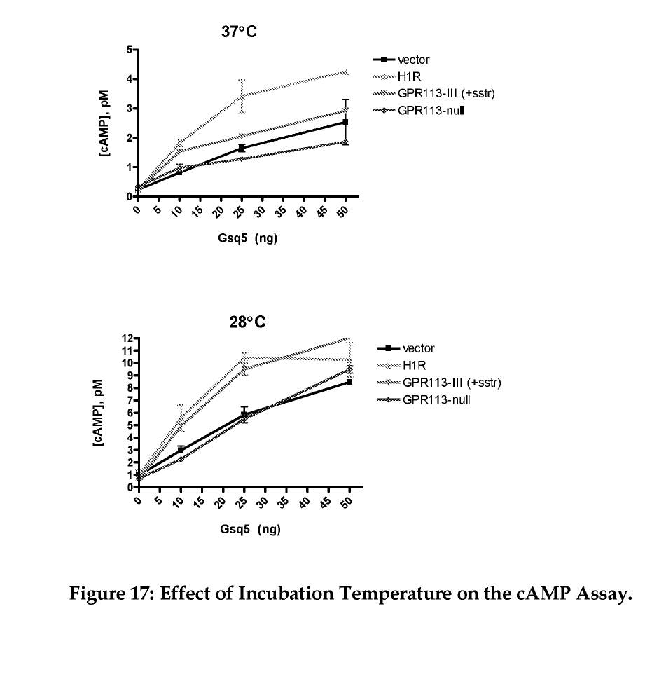

As shown in FIG. 17, additional experiments were conducted wherein GPR113 or control receptors were co-expressed with varying amounts of the Gsq5 chimeric G-protein and cAMP levels measured with the ELISA-based cAMP kit. Similar to the IP-One assay, cells were incubated at 37° C. for the 1st 24 hours after transfection followed by transfer of some cells to 28° C. for an additional 24 hrs before performing the assay. Consistent with the IP-One assay, the response of cells expressing GPR113-isoform III with the sstr tag increased relative to the negative controls with decreasing incubation temperature. The result is a larger assay window.

### Example 15

Co-Expression of GPR113 with Gs or Gsq5 Chimera

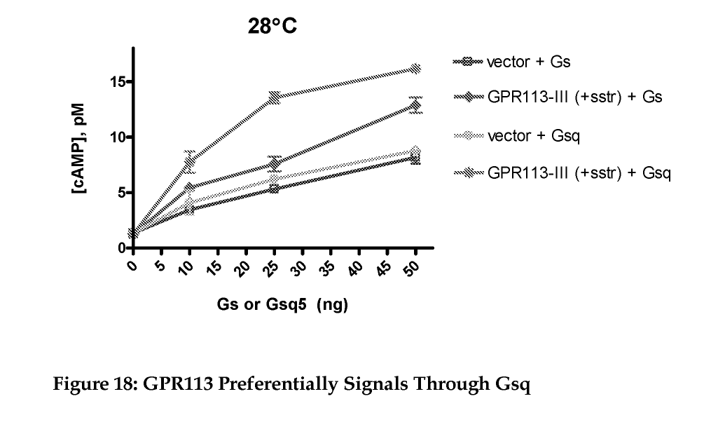

As shown in FIG. 18, additional experiments were conducted wherein GPR113 was co-expressed with varying amounts of Gs or the Gsq5 chimeric G-protein and cAMP levels measured with the ELISA-based cAMP kit. Cells were incubated at 28° C. prior to the assay.

The results as shown in FIG. 18 revealed that higher levels of cAMP were measured with Gsq5 vs Gs indicating that GPR113 preferentially signals through a Gq-mediated pathway.

### Example 16

### GPR113 Specificity

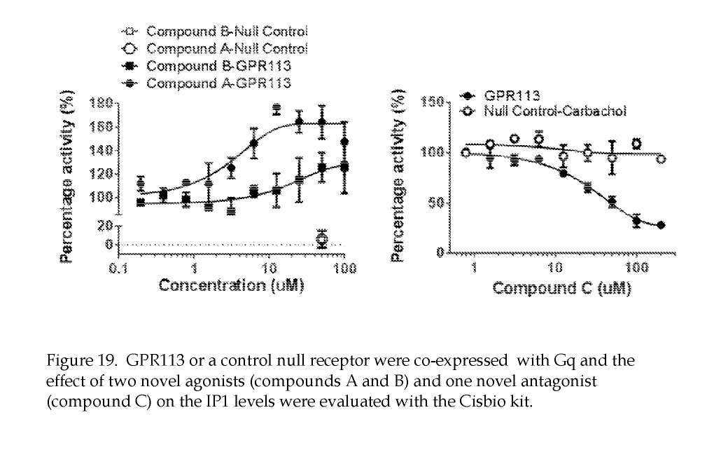

As shown in FIG. 19, two novel GPR113 agonists (compounds A and B) and one novel GPR113 antagonist (compound C) were identified by high throughput screening with cells co-expressing GPR113 and Gq and using the IPOne kit from Cisbio. The two agonists can dose-dependently increase levels of IP1 above those obtained by the constitutive activity of the receptor only in cells expressing GPR113 and not in the control cells. Conversely, the antagonist can dose-dependently decrease levels of IP1 below those obtained by the constitutive activity of the receptor. The antagonist shows specificity as it cannot decrease the carbachol-induced IP1 accumulation.

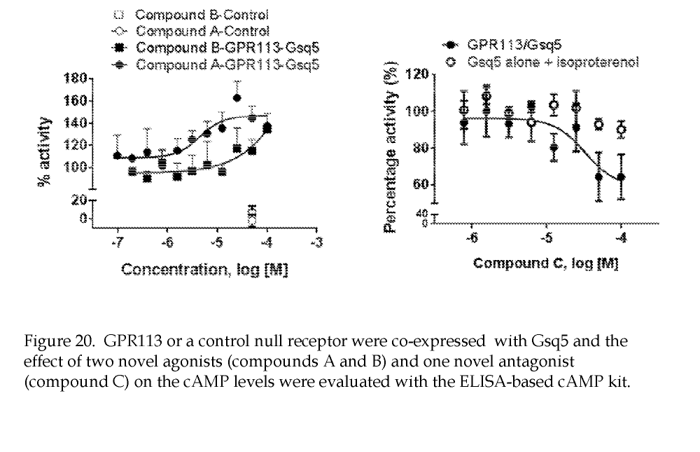

As shown in FIG. 20, the agonists and antagonists exhibited the same activity in a counter-screen where cells were expressing GPR113 and Gsq5, confirming the results described in FIG. 19.

### Applications of the Invention

Compounds which modulate, i.e., inhibit or enhance the activity of the subject fat taste specific gene and the GPR113 receptor polypeptide have important implications in mimicking fat taste or in modulating fat taste elicited by different fats such as oils, medium and long chain fatty acids, different lipids and the like.

In addition these compounds are potentially useful in therapeutic applications involving fat absorption and fat metabolism involving GPR113 expressing taste and other cells, potentially gastrointestinal cells expressing GPR113. These compounds may be useful in maintaining reduced fat diets and/or in controlling body weight. These compounds may be useful in treating diseases involving fat digestion and absorption as well as for the regulation of fat metabolism and the like. Such diseases may include diabetes, obesity, arteriosclerosis, hypercholesterolemia, hypercholesterolemia, disorders involving fat metabolism such as gallbladder disorders and fatty liver disease, and autoimmune diseases such as IBD.

### REFERENCES

All the references cited in this application are incorporated by reference in their entirety herein.

SEQUENCE LISTING

GPR113 Polypeptide Sequence (SEQ ID NO: 1)

| | |
|---|---|
| 1 | mvcsaaplll lattlpllgs pvaqasqpvs etgvrpregl qrrqwgplig rdkawnerid |
| 61 | rpfpacpipl sssfgrwpkg qtmwaqtstl tlteeelgqs qaggesgsgq lldgengage |
| 121 | salvsvyvhl dfpdktwppe lsrtltlpaa sasssprpll tglrlttecn vnhkgnfyca |
| 181 | clsgyqwnts iclhyppcqs lhnhqpcgcl vfshpepgyc qllppgspvt clpavpgiln |
| 241 | lnsqlqmpgd tlsltlhlsq eatnlswflr hpgspspill qpgtqvsvts shgqaalsys |
| 301 | nmshhwagey mscfeaqgfk wnlyevvrvp lkatdvarlp yqlsiscats pgfqlsccip |
| 361 | stnlaytaaw spgegskass fnesgsqcfv lavqrcpmad ttyacdlqsl glaplrvpis |
| 421 | itiiqdgdit cpedasvltw nvtkaghvaq apcpeskrgi vrrlcgadgv wgpvhssctd |
| 481 | arllalftrt kllqagqgsp aeevpqilaq lpgqaaeass psdlltllst mkyvakvvae |
| 541 | ariqldrral knlliatdkv ldmdtrslwt laqarkpwag stlllavetl acslcpqdhp |
| 601 | fafslpnvll qsqlfgptfp adysisfptr pplqaqiprh slaplvrngt eisitslvlr |
| 661 | kldhllpsny gqglgdslya tpglvlvisi magdrafsqg evimdfgntd gsphcvfwdh |
| 721 | slfqgrggws kegcqaqvas asptaqclcq hltafsvlms phtvpeepal alltqvglga |
| 781 | silallvclg vywlvwrvvv rnkisyfrha allnmvfcll aadtcflgap flspgprspl |
| 841 | claaaflchf lylatffwml aqalvlahql lfvfhqlakh rvlplmvllg ylcplglagv |
| 901 | tlglylpqgq ylregecwld gkggalytfv gpvlaiigvn glvlamamlk llrpslsegp |
| 961 | paekrqallg vikalliltp ifgltwglgl atlleevstv phyiftilnt lqgvfillfg |
| 1021 | clmdrkiqea lrkrfcraqa psstislvsc clqilscask smsegipwps sedmgtars |

GPR113 Genomic Sequence (SEQ ID NO: 2)

| | |
|---|---|
| 1 | tgggagctgg gaatgaggtg gaaacccagg acccagaaaa gagagggcag gtgcagcgag |
| 61 | ggagtggtgg cggagagaga ggactggctc tgatcacagt cggacaggtc tgtgaccagt |
| 121 | tctctagcgg agaggcctgg aaatgaactc atttgtcttt gaagcctcat ccataaaata |
| 181 | ggtgttgctg gacggatgac atgaagccgt gtatctgaag gcacagtgcc taggggagga |
| 241 | cttgctccct tcctgagccc tgtctatatg cacctggaca ggctgtggga gggggtctgc |
| 301 | tctgcattcc tgggactggc cagctaggtg agagaatcca gaggggaccg gcttgtggcc |
| 361 | tcgctgcctg tcctctccag ctgtcccctc tgctcctgta gaatcagcgc tggtctccgt |
| 421 | ctatgtacat ctggactttc cagataagac ctggccccct gaactctcca ggacactgac |
| 481 | tctccctgct gcctcagctt cctcttcccc aaggcctctt ctcactggcc tcagactcac |
| 541 | aacaggtacc acttgcgtgg gaagggggct gagagtgaat gaacataggc tcccgggcct |
| 601 | cctgcagcca gcttgcctga gactctgtga gcccctctgt atttcctgga ggaagggctg |
| 661 | cctggttctg tctccgtggc ccagctcctt cctcacctcc ctaccagaca gacccttcct |
| 721 | tgcctgccac atccccctat cttctaactt tggctgatgg cccaagggac agacaacgtg |
| 781 | ggcccagacc tccaccttca cctgttccct ggcccccgag acatctgctg cttcgagtcc |
| 841 | tgactgagga ggcagtcctg atgcatgggc ctgactgagg cacctgtagc ttggggattg |
| 901 | gtccagatac ccagccctaa agcctctcag gcatcaggca ggtgtctgcc ctgcccacct |
| 961 | agcttcttca gacagcctgc ccaccccctc ttctcttctc tctgtcagag tgtaatgtca |
| 1021 | accacaaggg gaatttctat tgtgcttgcc tctctggcta ccagtggaac accagcatct |
| 1081 | gcctccatta ccctccttgt caaagcctcc acaaccacca gccttgtggc tgccttgtct |
| 1141 | tcagccatcc cgaacccggg tactgccagt tgctgccacc tggtgaggaa ggttgggaac |
| 1201 | ttggaaacca atggccttaa gtgaaataaa tgttctcagt ggttttctcc tctctgaacc |
| 1261 | tgtagtttgg ccagctggtc caagcacagc tgctcctctg ggtgggagaa aaagccagcc |
| 1321 | atcatagcag atcacaggcc ctgagcttgg aacctgagta gggagactaa tgagagaggc |
| 1381 | cccagagaca taaggaccag gagagaaagt gctggagtga ctgcttttta ccttaggagg |
| 1441 | caggaagcag ctccagtagc ccaggatacc tgggggaggg agaggcatag accaaaaagg |
| 1501 | ttccctcttt ggtttccaat aacagataga gtcttccagg ctggattgca gcagccacat |
| 1561 | tcaggtgccc acccagggac aaaaagaaaa agttaaaaag ctagggaggg agtgtggagg |
| 1621 | aatgggctcc agagtcaggg gagaagccat tgctcggctg catctgaggg ccataagtcc |
| 1681 | ctcctccagg gtcccctgtc acctgcctcc ctgcagtccc cgggatcctc aacctgaact |
| 1741 | cccagctgca gatgcctggt gacacgctga gcctgactct ccatctgagc caggaggcca |
| 1801 | ccaacctgag ctggttcctg aggcacccag ggagccccag tcccatcctc ctgcagccag |
| 1861 | ggacacaggt gtctgtgact tccagccacg gccaggctgc cctcagcgtc tccaacatgt |
| 1921 | cccatcactg ggcaggtagc cagcctgtcc tctccttgcc tcctttctcc ttcctcttac |
| 1981 | ttcccttcat cctcgtcttc cttctctgct ttccttcacc tcttcttccc acgcctccct |
| 2041 | cccttctcct tccttctttt ctttccacct ctttctcacc cttttcatct ttccatttac |
| 2101 | ccattctggg gaaacaaagg ctaagaggtc ccttggtgtg aaaaattgca atgtggaaaa |
| 2161 | ttctaaaaat ggccagctgt tttcactgtg gtctgggact tctgagaccc ttttcagggt |
| 2221 | ttacaaagtc acaactattg tcctaatatg ctaagatgtc atttgaccct ttcactccca |
| 2281 | ctccctcagg tgtagacagt ggccctttcc agaggctaca gggccatcac gagattgaat |
| 2341 | gcaaatgcag atgggagaac ccagacacgg gcaagatttg caaacatgta aaacaaagtc |
| 2401 | acttgtctaa ttatgttttg gaaaatgtag ttatttttca taaaaatgtt tctgttaaca |
| 2461 | aaaatactac aattctccac acaaaatatg gagaatgtgg agaataccgt ctcaatgtct |
| 2521 | gctgagaaca gatccatgtt tttcaagatg ctaaaatggc aggggtggtg caggaagggc |
| 2581 | atctgctcta gggagagcat gaaattcacg ggcatgggcc gataaaagag agatctcttc |
| 2641 | tacctcctag aaatccttct tggggacagg gaatgtccac caaaggggcc atcctgggac |
| 2701 | cttgcttgct ggggttaagc actgggtggc aggcagagga caggagcaag gctgtggctt |
| 2761 | ggaaagcagc agagattctg tggtgcagcg gggcccagag gagccacata gcgccgcaca |
| 2821 | cacgtttctg caggtgagta catgagctgc ttcgaggccc agggcttcaa gtggaacctg |
| 2881 | tatgaggtgg tgagggtgcc cttgaaggcg acagatgtgg ctcgacttcc ataccagctg |
| 2941 | tccatctcct gtgccacctc ccctggcttc cagctgagct gctgcatccc cagcacaaac |
| 3001 | ctggcctaca ccgcggcctg gagccctgga gagggcagca aaggtatgag aaggggccag |
| 3061 | cagtcagggg tcagagggac cagggggcag ctgtctcttc caggcagctg ggtcttcagc |
| 3121 | tcatgagaaa cagaggccac agttcaacca gagagtgggg tccaaggcca acactgtttt |
| 3181 | ctaccccatc agagccatgc cacgtctatt gccataacat aaccacatgt gtataggaaa |
| 3241 | cttttgcaaa atgctgtcat ctacacaatc tcatttaact ctctatggaa ttagtttgat |
| 3301 | ggtagtctcc attttacaaa tgaggaaatg gtggaaactg agtcctagag cttgttagag |
| 3361 | accccacagt cccctccagc aaaatccaag ctctcttcct ctgtccaagt ggagcccaca |
| 3421 | catcatttgg ctcttcccca ctgcttcctc tgtttctgaa ttgctagaaa gactgaaaca |
| 3481 | gcatgtcaga gcctgctggg ttccaggcct gtccctggcc caatgacagt tcccttcttc |
| 3541 | gttttgcctt cagcttcctc cttcaacgag tcaggctctc agtgctttgt gctggctgtt |
| 3601 | cagcgctgcc cgatggctga caccacgtac gcttgtgacc tgcagagcct gggcctggct |
| 3661 | ccactcaggg tccccatctc catcaccatc atccagggta cgcagggcct ggggcccagt |
| 3721 | gggctggtcc cagctgcttg ccttgggagc acgggctctc ttgcatggca cgtctctgcc |
| 3781 | ctgggcaaca ggaccaggct tcggggcccg catagggttc tgcccaagga gaggctcagg |
| 3841 | tgaggctgtg attgctgagt agcgcctgct cgtcattctt cagatggaga catcacctgc |
| 3901 | cctgaggacg cctcggtgct cacctggaat gtcaccaagg ctggccacgt ggcacaggcc |
| 3961 | ccatgtcctg agagcaagag gggcatagtg aggaggctct gtggggctga cggagtctgg |
| 4021 | gggccggtcc acagcagctg cacagatgcg aggctcctgg ccttgttcac tagaaccaag |
| 4081 | gtgaagcttc caccctgctg cccacgtgcc ccctccacgg cccaccctag cctctctagg |
| 4141 | acccagcttg cagacccttt tccccaaggc ccagcccaca ggctgttcag cttctctgaa |
| 4201 | gtggagccct agcagagcca ggaagtagga gtgagagggc ttctgggggt caacaatctc |
| 4261 | catgggtctg ggatgctctt ctcaaaccat cattccacca tgtgtcccac ttcatgctgt |
| 4321 | ctcgtctgtc tcagctgctg caggcaggcc agggcagtcc tgctgaggag gtgccacaga |
| 4381 | tcctggcaca gctgccaggg caggcggcag aggcaagttc accctccgac ttactgaccc |
| 4441 | tgctgagcac catgaaatac gtggccaagg tggtggcaga ggccagaata cagcttgacc |
| 4501 | gcagagccct gaaggtgaga tctctgagcc acagtggggg ccagctgggc agtcgggggc |
| 4561 | tgaagactcc ccacctgtgg gcatttctgt ccctctgatg tcaccatggg ctgttgggca |
| 4621 | gcagaccttt ccagagtcca ggggcctgct cctgatccat ttctcctctc agacaccact |
| 4681 | ctctgaggct gcagaatgga ggcctggcgc tgggagcaca tgggggttgg aggcaggcaa |
| 4741 | gggtgtggag acatgaggcc cgaggcgtgt gtgcgcatgc aggcgtgtgg ctatgataca |
| 4801 | gacaggaagt ttctatggag acgctgaagt atgcttggct ttgctgggct cacctaaatc |
| 4861 | ggctctctgt atgggcatcc attggtgacc catgagctgc agccaaaagt gtaacaaagg |
| 4921 | gcaatgatat tacacaccgt ttatgcctgg gaatacatgg catgtgtgaa tgcacagaca |
| 4981 | tgcgtgtggc cgtcgcctcc aggacacggt gccctctacc actgctggtc accattccta |
| 5041 | gctttgcaga cctggagggg ccaaagaatg ggagaagtcc cctcttagaa cctgggtggc |
| 5101 | ccctagggat ggagggggaa gaagggtttt cagcagaggg gctgggtgca ggtcagggga |
| 5161 | catatccttg aagatgcccc aggtggttgg ccaaacagct ccctgttctt cccatctaga |
| 5221 | aagtctccct tcacaggcct gtcttcctct cccttttctc tccaaccttg ggtcgcacac |
| 5281 | tggactggga agggaaggtg tggggtctgt tgttctcatt gcccccggct cagtcctgtg |
| 5341 | ggcgccagca gacggggttc atctttcttt tgggtgctgc agaatctcct gattgccaca |
| 5401 | gacaaggtcc tagatatgga caccaggtct ctgtggaccc tggcccaagc ccggaagccc |
| 5461 | tgggcaggct cgactctcct gctggctgtg gagaccctgg catgcagcct gtgcccacag |
| 5521 | gaccacccct tcgccttcag cttacccaat gtgctgctgc agagccagct gtttggaccc |
| 5581 | acgtttcctg ctgactacag catctccttc cctactcggc ccccactgca ggctcagatt |
| 5641 | cccaggcact cactggcccc attggtccgt aatggaactg aaataagtat tactagcctg |
| 5701 | gtgctgcgaa aactggacca ccttctgccc tcaaactatg gacaagggct gggggattcc |
| 5761 | ctctatgcca ctcctggcct ggtccttgtc atttccatca tggcaggtga ccgggccttc |
| 5821 | agccagggag aggtcatcat ggactttggg aacacagatg gttcccctca ctgtgtcttc |
| 5881 | tgggatcaca gtctcttcca gggcaggggg ggttggtcca aagaagggtg ccaggcacag |
| 5941 | gtggccagtg ccagccccac tgctcagtgc ctctgccagc acctcactgc cttctccgtc |
| 6001 | ctcatgtccc cacacactgt tccggaagaa cccgctctgg cgctgctgac tcaagtgggc |
| 6061 | ttgggagctt ccatactggc gctgcttgtg tgcctgggtg tgtactggct ggtgtggaga |
| 6121 | gtcgtggtgc ggaacaagat ctcctatttc cgccacgccg ccctgctcaa catggtgttc |
| 6181 | tgcttgctgg ccgcagacac ttgcttcctg ggcgccccat tcctctctcc agggccccga |
| 6241 | agcccgctct gccttgctgc cgccttcctc tgtcatttcc tctacctggc cacctttttc |
| 6301 | tggatgctgg cgcaggccct ggtgttggcc caccagctgc tctttgtctt tcaccagctg |
| 6361 | gcaaagcacc gagttctccc cctcatggtg ctcctgggct acctgtgccc actggggttg |
| 6421 | gcaggtgtca ccctggggct ctacctacct caagggcaat acctgaggga gggggaatgc |
| 6481 | tggttggatg ggaagggagg ggcgttatac accttcgtgg ggccagtgct ggccatcata |
| 6541 | ggcgtgaatg ggctggtact agccatggcc atgctgaagt tgctgagacc ttcgctgtca |
| 6601 | gagggacccc cagcagagaa gcgccaagct ctgctggggg tgatcaaagc cctgctcatt |
| 6661 | cttacaccca tctttggcct cacctggggg ctgggcctgg ccactctgtt agaggaagtc |
| 6721 | tccacggtcc ctcattacat cttcaccatt ctcaacaccc tccaggtagg tgataggggg |
| 6781 | gtggctgtgt tttttgcttt tttagatggt ctaagtcact gccgatctct tctctaggag |
| 6841 | gtaccaaggt ggagcagaag aaacataggt tcaggaattt tggaaggctt aggtgtggat |
| 6901 | cccagttcct ccactgagta gctggataac tttggacaaa ttacataacc tctctgagct |
| 6961 | ttggttttct tatctgtaaa ataatagctg attttgttgg agaaatcagg aaattgtcag |
| 7021 | tacccaatcc tttgctatcc cttttataac cataacaata agaaaagcac ctgaaatgga |
| 7081 | tcctatgcac caaatagtgg taacagaaaa attgagatga gaagccttag gatgtgaatt |
| 7141 | acacaggaca gaaggagcat gttgattcgg gtggatccct tcctccttga ccagcttatc |
| 7201 | cccatgtccc tcttctcagg gcgtcttcat cctattgttt ggttgcctca tggacaggaa |
| 7261 | ggtaagtctg cccacctaac cccctgcctc acttgcagcc cgcaggccgg ggccgtggct |
| 7321 | ggcataagca gagcatttac ctctcccgca gatacaagaa gctttgcgca aacgcttctg |
| 7381 | ccgcgcccaa gcccccagct ccaccatctc cctggtgagt tgctgccttc agatcctcag |
| 7441 | ctgtgcatcc aagagcatgt cagaaggcat tccatggccc tcctcagagg acatgggcac |
| 7501 | agccagaagc tgagagaaga ttggggttgt tttttagaat gaacagtttt ccggttccag |
| 7561 | ctccccacca gtggaatgag cagcctggtc agagcagtca ggatcagggt cctgggttcc |
| 7621 | tgattatcac ctggactcct gctgactctc ttttctctgg tttctccatc taaaaatctg |
| 7681 | cctccagtta gcatttgaag gaaaagtgtg ggatcagtac tcatgggagt tactgtagct |
| 7741 | gagagcaaaa tttctaggat tcctgcagca caggcaggag tgcatgtgag aaagtaaaac |
| 7801 | agatacaacc tcttcaaggg agagttgaca atactaataa ctgccctgca attgggcctt |
| 7861 | cccacccctt cct |
| // | |

GPR113 mRNA Sequence (SEQ ID NO: 3)

| | |
|---|---|
| 1 | atcagcagga tggcatcggc aagtcgctcc cctcccgggc ctcatctgcc aaacgatcat |
| 61 | ctcctcctcc gaagttgtat gcatgacagg cgagtggaaa cttcactaaa atgaaggcga |
| 121 | ttgacacaac agaaggaact ccatcctttc gggggcttac gaaaataata agtttaaaaa |
| 181 | aaataggaag ggaattccct cgctccatga tcactgagcg ctctcctaag gaaaaggaaa |
| 241 | tctcccgggg ggtgccgact acgggcggcg ggcttaggat gctcccacgc tccccgaccc |
| 301 | ccaatcccca ggacccgcag gacctccgga ggaacgcccg ccagcccgcc cggagccacg |
| 361 | cggcacaagg tgacacggac cgcgccgcgc gggcccctca gccgcctggg cgaggccggg |
| 421 | agcagggaga ggggcatccg ccggcccgcg gtaccttgta cttatcaaag ccagccagct |
| 481 | gctccgggct cacgtattcg tagccagcca tgacgacccg aaaactgagc gcccactcgg |
| 541 | cagcgactcc cggctacaag gctgtgacac acaagcacca caccggctgg gcaaggatgg |
| 601 | caaagactgg gctgcccgag aagcttcctc cttcaacgag tcaggctctc agtgctttgt |
| 661 | gctggctgtt cagcgctgcc cgatggctga caccacgtac gcttgtgacc tgcagagcct |
| 721 | gggcctggct ccactcaggg tccccatctc catcaccatc atccaggatg gagacatcac |
| 781 | ctgccctgag gacgcctcgg tgctcacctg gaatgtcacc aaggctggcc acgtggcaca |
| 841 | ggccccatgt cctgagagca agaggggcat agtgaggagg ctctgtgggg ctgacggagt |
| 901 | ctgggggccc gtccacagca gctgcacaga tgcgaggctc ctggccttgt tcactagaac |
| 961 | caagctgctg caggcaggcc agggcagtcc tgctgaggag gtgccacaga tcctggcaca |
| 1021 | gctgccaggg caggcggcag aggcaagttc accctccgac ttactgaccc tgctgagcac |
| 1081 | catgaaatac gtggccaagg tggtggcaga ggccagaata cagcttgacc gcagagccct |
| 1141 | gaagaatctc ctgattgcca cagacaaggt cctagatatg gacaccaggt ctctgtggac |
| 1201 | cctggcccaa gcccggaagc cctgggcagg ctcgactctc ctgctggctg tggagaccct |
| 1261 | ggcatgcagc ctgtgcccac aggactaccc cttcgccttc agcttaccca atgtgctgct |
| 1321 | gcagagccag ctgtttggac ccacgtttcc tgctgactac agcatctcct tccctactcg |
| 1381 | gcccccactg caggctcaga ttcccaggca ctcactggcc ccattggtcc gtaatggaac |
| 1441 | tgaaataagt attactagcc tggtgctgcg aaaactggac caccttctgc cctcaaacta |
| 1501 | tggacaaggg ctgggggatt ccctctatgc cactcctggc ctggtccttg tcatttccat |
| 1561 | catggcaggt gaccgggcct tcagccaggg agaggtcatc atggactttg ggaacacaga |
| 1621 | tggttcccct cactgtgtct tctgggatca cagtctcttc cagggcaggg ggggttggtc |
| 1681 | caaagaaggg tgccaggcac aggtggccag tgccagcccc actgctcagt gcctctgcca |
| 1741 | gcacctcact gccttctccg tcctcatgtc cccacacact gttccggaag aacccgctct |
| 1801 | ggcgctgctg actcaagtgg gcttgggagc ttccatactg gcgctgcttg tgtgcctggg |
| 1861 | tgtgtactgg ctggtgtgga gagtcgtggt gcggaacaag atctcctatt tccgccacgc |
| 1921 | cgccctgctc aacatggtgt tctgcttgct ggccgcagac acttgcttcc tgggcgcccc |
| 1981 | attcctctct ccagggcccc gaagcccgct ctgccttgct gccgccttcc tctgtcattt |
| 2041 | cctctacctg gccacctttt tctggatgct ggcgcaggcc ctggtgttgg cccaccagct |
| 2101 | gctctttgtc tttcaccagc tggcaaagca ccgagttctc cccctcatgg tgctcctggg |
| 2161 | ctacctgtgc ccactggggt tggcaggtgt caccctgggg ctctacctac ctcaagggca |
| 2221 | atacctgagg gagggggaat gctggttgga tgggaaggga ggggcgttat acaccttcgt |
| 2281 | ggggccagtg ctggccatca taggcgtgaa tgggctggta ctagccatgg ccatgctgaa |
| 2341 | gttgctgaga ccttcgctgt cagagggacc cccagcagag aagcgccaag ctctgctggg |
| 2401 | ggtgatcaaa gccctgctca ttcttacacc catctttggc ctcacctggg ggctgggcct |
| 2461 | ggccactctg ttagaggaag tctccacggt ccctcattac atcttcacca ttctcaacac |
| 2521 | cctccagggc gtcttcatcc tattgtttgg ttgcctcatg gacaggaaga tacaagaagc |
| 2581 | tttgcgcaaa cgcttctgcc gcgcccaagc ccccagctcc accatctccc tggccacaaa |
| 2641 | tgaaggctgc atcttggaac acagcaaagg aggaagcgac actgccagga agacagatgc |
| 2701 | ttcagagtga accacacacg gacccatgtt cctgcaaggg agttgaggct gtgtgcttga |
| 2761 | acccaccaga tgagccctgg cccaatgctc tgaactcttc ccgcctcccg gagctcagcc |
| 2821 | cttgagaaag gcaggcttat atttccctta gtgacactca tttatcttac agctcacccc |
| 2881 | ttctcatttc taaagtatcc agcaagaata gcaggaaaaa ttagctaaag gcacctaatg |
| 2941 | aataagcctg cctttgctcc agaaataatc gacagatatc aaagtgcgga ataattacaa |
| 3001 | gtaaactttc tcaaccagtt tttaactaca acaatacatg ttgtgaatga atatatttga |
| 3061 | taaaaatggt tttaattgac ctattcagcg atttctgatt atttcttttt caatagttat |
| 3121 | gaagaaagga tgacttactt gacaggaacc tctgatcttt caaacattgg agatgaaggg |
| 3181 | cagaatttgg tttgtctttt caagtttagg aaaaggtgaa gttaattggt ccctctttct |
| 3241 | ttaaccttta aaaaatcaat ataaaatgta agtttcttaa ccatatccat gtatagaggc |
| 3301 | attgattgat atgagcacgt tgtaagaata ggttataaaa atttaaagtt taatataaat |
| 3361 | ttatatcaat taataaagtt taatttatat ttaaaaatga atactagaag aaaatctttt |
| 3421 | tgaagacacc aagatatcta tctggctgaa ttaacttatg gaattcacaa gaggaagatg |
| 3481 | acaggattct gagaaatttt taaactagat acgtgaaaaa agtctgatga atcggtcttt |
| 3541 | gttaattatg caattcatgg atatttttta taaaatggga cgggggcatt ttctgttaaa |
| 3601 | ataaaaatgg ttatgctatc |

GPR113 Nucleotide Sequence (3240 nt) SEQ ID NO: 4)
ATGGTCTGTTCGGCTGCCCCACTGCTGCTCCTGGCCACAACTCTTCCCCTGCTGGGGTCACCAGTTGCCC
AAGCATCCCAACCTGTAAGTGAGACTGGGGTGAGACCCAGGGAAGGTCTGCAGAGGCGACAATGGGGACC
CCTGATTGGGAGAGACAAAGCATGGAATGAAAGGATAGACAGACCCTTCCCTGCCTGCCCCATCCCCCTA
TCTTCTAGCTTTGGCCGATGGCCCAAGGGCCAGACAATGTGGGCCCAGACCTCCACCCTCACCCTGACAG
AGGAGGAGTTGGGACAGAGTCAGGCTGGAGGGGAATCTGGATCTGGGCAGCTCCTGGACCAAGAGAATGG
AGCAGGGGAATCAGCGCTGGTCTCCGTCTATGTACATCTGGACTTTCCAGATAAGACCTGGCCCCCTGAA
CTCTCCAGGACACTGACTCTCCCTGCTGCCTCAGCTTCCTCTTCCCCAAGGCCTCTTCTCACTGGCCTCA
GACTCACAACAGAGTGTAATGTCAACCACAAGGGGAATTTCTATTGTGCTTGCCTCTCTGGCTACCAGTG
GAACACCAGCATCTGCCTCCATTACCCTCCTTGTCAAAGCCTCCACAACCACCAGCCTTGTGGCTGCCTT
GTCTTCAGCCATCCCGAACCCGGGTACTGCCAGTTGCTGCCACCTGGGTCCCCTGTCACCTGCCTCCCTG
CAGTCCCCGGGATCCTCAACCTGAACTCCCAGCTGCAGATGCCTGGTGACACGCTGAGCCTGACTCTCCA
TCTGAGCCAGGAGGCCACCAACCTGAGCTGGTTCCTGAGGCACCCAGGGAGCCCCAGTCCCATCCTCCTG
CAGCCAGGGACACAGGTGTCTGTGACTTCCAGCCACGGCCAGGCTGCCCTCAGCGTCTCCAACATGTCCC
ATCACTGGGCAGGTGAGTACATGAGCTGCTTCGAGGCCCAGGGCTTCAAGTGGAACCTGTATGAGGTGGT
GAGGGTGCCCTTGAAGGCGACAGATGTGGCTCGACTTCCATACCAGCTGTCCATCTCCTGTGCCACCTCC
CCTGGCTTCCAGCTGAGCTGCTGCATCCCCAGCACAAACCTGGCCTACACCGCGGCCTGGAGCCCTGGAG
AGGGCAGCAAAGCTTCCTCCTTCAACGAGTCAGGCTCTCAGTGCTTTGTGCTGGCTGTTCAGCGCTGCCC
GATGGCTGACACCACGTACGCTTGTGACCTGCAGAGCCTGGGCCTGGCTCCACTCAGGGTCCCCATCTCC
ATCACCATCATCCAGGATGGAGACATCACCTGCCCTGAGGACGCCTCGGTGCTCACCTGGAATGTCACCA
AGGCTGGCCACGTGGCACAGGCCCCATGTCCTGAGAGCAAGAGGGGCATAGTGAGGAGGCTCTGTGGGGC
TGACGGAGTCTGGGGGCCGGTCCACAGCAGCTGCACAGATGCGAGGCTCCTGGCCTTGTTCACTAGAACC
AAGCTGCTGCAGGCAGGCCAGGGCAGTCCTGCTGAGGAGGTGCCACAGATCCTGGCACAGCTGCCAGGGC
AGGCGGCAGAGGCAAGTTCACCCTCCGACTTACTGACCCTGCTGAGCACCATGAAATACGTGGCCAAGGT
GGTGGCAGAGGCCAGAATACAGCTTGACCGCAGAGCCCTGAAGAATCTCCTGATTGCCACAGACAAGGTC
CTAGATATGGACACCAGGTCTCTGTGGACCCTGGCCCAAGCCCGGAAGCCCTGGGCAGGCTCGACTCTCC
TGCTGGCTGTGGAGACCCTGGCATGCAGCCTGTGCCCACAGGACCACCCCTTCGCCTTCAGCTTACCCAA
TGTGCTGCTGCAGAGCCAGCTGTTTGGACCCACGTTTCCTGCTGACTACAGCATCTCCTTCCCTACTCGG
CCCCCACTGCAGGCTCAGATTCCCAGGCACTCACTGGCCCCATTGGTCCGTAATGGAACTGAAATAAGTA
TTACTAGCCTGGTGCTGCGAAAACTGGACCACCTTCTGCCCTCAAACTATGGACAAGGGCTGGGGGATTC
CCTCTATGCCACTCCTGGCCTGGTCCTTGTCATTTCCATCATGGCAGGTGACCGGGCCTTCAGCCAGGGA
GAGGTCATCATGGACTTTGGGAACACAGATGGTTCCCCTCACTGTGTCTTCTGGGATCACAGTCTCTTCC
AGGGCAGGGGGGGTTGGTCCAAAGAAGGGTGCCAGGCACAGGTGGCCAGTGCCAGCCCCACTGCTCAGTG
CCTCTGCCAGCACCTCACTGCCTTCTCCGTCCTCATGTCCCCACACACTGTTCCGGAAGAACCCGCTCTG
GCGCTGCTGACTCAAGTGGGCTTGGGAGCTTCCATACTGGCGCTGCTTGTGTGCCTGGGTGTGTACTGGC
TGGTGTGGAGAGTCGTGGTGCGGAACAAGATCTCCTATTTCCGCCACGCCGCCCTGCTCAACATGGTGTT
CTGCTTGCTGGCCGCAGACACTTGCTTCCTGGGCGCCCCATTCCTCTCTCCAGGGCCCCGAAGCCCGCTC
TGCCTTGCTGCCGCCTTCCTCTGTCATTTCCTCTACCTGGCCACCTTTTTCTGGATGCTGGCGCAGGCCC
TGGTGTTGGCCCACCAGCTGCTCTTTGTCTTTCACCAGCTGGCAAAGCACCGAGTTCTCCCCCTCATGGT
GCTCCTGGGCTACCTGTGCCCACTGGGGTTGGCAGGTGTCACCCTGGGGCTCTACCTACCTCAAGGGCAA
TACCTGAGGGAGGGGGAATGCTGGTTGGATGGGAAGGGAGGGGCGTTATACACCTTCGTGGGGCCAGTGC
TGGCCATCATAGGCGTGAATGGGCTGGTACTAGCCATGGCCATGCTGAAGTTGCTGAGACCTTCGCTGTC
AGAGGGACCCCCAGCAGAGAAGCGCCAAGCTCTGCTGGGGGTGATCAAAGCCCTGCTCATTCTTACACCC
ATCTTTGGCCTCACCTGGGGGCTGGGCCTGGCCACTCTGTTAGAGGAAGTCTCCACGGTCCCTCATTACA
ACAAGAAGCTTTGCGCAAACGCTTCTGCCGCGCCCAAGCCCCCAGCTCCACCATCTCCCTGGTGAGTTGC
TGCCTTCAGATCCTCAGCTGTGCATCCAAGAGCATGTCAGAAGGCATTCCATGGCCCTCCTCAGAGGACA
TGGGCACAGCCAGAAGCTGA
GPR113 Translation (1079 aa)(SEQ ID NO: 5):
MVCSAAPLLLLATTLPLLGSPVAQASQPVSETGVRPREGLQRRQWGPLIGRDKAWNERIDRPFPACPIPL
SSSFGRWPKGQTMWAQTSTLTLTEEELGQSQAGGESGSGQLLDQENGAGESALVSVYVHLDFPDKTWPPE
LSPTLTLPAASASSSPPPLLTGLRLTTECNVNHKGNFYCACLSGYQWNTSICLHYPPCQSLHNHQPCGCL
VFSHPEPGYCQLLPPGSPVTCLPAVPGILNLNSQLQMPGDTLSLTLHLSQEATNLSWFLRHPGSPSPILL
QPGTQVSVTSSHGQAALSVSNMSHHWAGEYMSCFEAQGFKWNLYEVVRVPLKATDVARLPYQLSISCATS
PGFQLSCCIPSTNLAYTAAWSPGEGSKASSFNESGSQCFVLAVQRCPMADTTYACDLQSLGLAPLRVPTS
ITIIQDGDITCPEDASVLTWNVTKAGHVAQAPCPESKRGIVRRLCGADGVWGPVHSSCTDARLLALFTRT
KLLQAGQGSPAEEVPQILAQLPGQAAEASSPSDLLTLLSTMKYVAKVVAEARIQLDPRALKNLLIATDKV
LDMDTRSLWTLAQARKPWAGSTLLLAVETLACSLCPQDHPFAFSLPNVLLQSQLFGPTFPADYSISFPTR
PPLQAQIPRHSLAPLVRNGTEISITSLVLRKLDHLLPSNYGQGLGDSLYATPGLVLVISIMAGDRAFSQG
EVIMDFGHTDGSPHCVFWDHSLFQGRGGWSKEGCQAQVASASPTAQCLCQHLTAFSVLMSPHTVPEEPAL
ALLTQVGLGASILALLVCLGVYWLVWRVVVRNKISYFRHAALLNMVFCLLAADTCFLGAPFLSPGPRSPL
CLAAAFLCHFLYLATFFWMLAQALVLAHQLLFVFHQLAKHRVLPLMVLLGYLCPLGLAGVTLGLYLPQGQ
YLREGECWLDGKGGALYTFVGPVLAIIGVNGLVLAMAMLKLLRPSLSEGPPAEKRQALLGVIKALLILTP
IFGLTWGLGLATLLEEVSTVPHYIFTILNTLQGVFILLFGCLMDRKIQEALRKRFCRAQAPSSTISLVSC
CLQILSCASKSMSEGIPWPSSEDMGTARS

1. A method for eliciting, mimicking, blocking, enhancing or modulating fat, lipid, or fatty acid associated taste (“fat taste”) comprising administering to a subject an effective amount of a compound that binds to a GPR113 polypeptide and/or modulates the activity of GPR113.

2. The method of claim 1 wherein:
(i) the GPR113 modulator blocks or inhibits GPR113 activity;
(ii) the GPR113 modulator enhances or agonizes GPR113 activity; or
(iii) the GPR113 modulator is a naturally occurring or synthetic compound.

3-5. (canceled)

6. A method for identifying a compound suitable for eliciting, mimicking, blocking, enhancing or modulating fat, lipid, or fatty acid associated taste (“fat taste”) comprising the following:
(i) contacting an isolated GPR113 receptor or a cell that expresses a nucleic acid encoding a human GPR113 receptor polypeptide or a chimera or fragment thereof or an ortholog or a nucleic acid encoding a polypeptide possessing at least 90% sequence identity to the polypeptide encoded thereby with at least one putative modulator compound;
(ii) detecting whether said compound binds or modulates the binding of another ligand to said GPR113 polypeptide or modulates signal transduction elicited by said GPR113 polypeptide; and
(iii) identifying the compound as a potential fat taste modulator based on whether it specifically binds or modulates the specific binding of another ligand to said GPR113 polypeptide or specifically modulates the signal transduction of said GPR113 polypeptide.

7. The assay of claim 6 wherein:
(i) the cell additionally expresses a G protein that functionally couples to said GPR113 polypeptide;
(ii) the cell additionally expresses a G protein that functionally couples to said GPR113 polypeptide selected from Gi proteins, Gq proteins, Gs proteins, Ga15, Ga16, transducin, gustducin or a chimera of any of the foregoing;
(iii) the cell additionally expresses a G protein that functionally couples to said GPR113 polypeptide which comprises a chimera of a Gs and Gq;
(iv) the cell additionally expresses a G protein that functionally couples to said GPR113 polypeptide which comprises a chimeric G protein which consists of a Gs protein wherein at least the last 5-40 amino acids are substituted with those of Gq;
(v) the cell additionally expresses a G protein that functionally couples to said GPR113 polypeptide which is a chimeric G protein which consists of a Gq protein wherein at least the last 5-40 amino acids are substituted with those of Gs;
(vi) the assay includes the use of a detectable label;
(vii) the assay uses a mammalian cell which endogenously or recombinantly expresses GPR113;
(viii) the assay uses a GPR113-expressing cell further expresses T1R3, GPR40, GPR120, CD36, phospholipase-Cβ2, and/or TRPM5;
(ix) the assay uses a human or non-human primate cell that endogenously expresses GPR113;
(x) the assay uses an enzyme, radionuclide, chemiluminescent compound or fluorescent compound label;
(xi) the assay detects the displacement of a labeled ligand from said such receptor;
(xii) the assay is a fluorescence polarization or FRET assay;
(xiii) the assay detects conformational changes in the receptor based on altered susceptibility to proteolysis;
(xiv) the assay is a competitive binding assay;
(xv) the assay is a non-competitive binding assay;
(xvi) the assay detects the effect of said compound on the specific binding of another compound to said receptor;
(xvii) the assay uses an intact or permeabilized GPR113-expressing cell;
(xviii) the assay uses a membrane extract which comprises said receptor;
(xix) the receptor is expressed on the surface of said cell;
(xx) the assay uses a GPR113-expressing eukaryotic cell;
(xxi) the assay uses a GPR113-expressing prokaryotic cell;
(xxii) the assay uses a GPR113-expressing yeast, insect, amphibian or mammalian cell;
(xxiii) the assay uses a GPR113-expressing CHO cell, COS cell, BHK cell, VERO cell, HT1080 cell, MRC-5 cell, WI 38 cell, MDCK cell, MDBK cell, 293 cell, 293T cell, RD cell, a COS-7 cell, Jurkat cell, HUT cell, SUPT cell, C8166 cell, MOLT4/clone 8 cell, MT-2 cell, MT-4 cell, H9 cell, PM1 cell, CEM cell, a myeloma cell, SB20 cell, LtK cell, HeLa cell, WI-38 cell, L2 cell, CMT-93 cell, CEMX 174 cell or Xenopus oocyte;
(xxiv) the assay uses a GPR113-expressing cell that endogenously expresses said GPR113 polypeptide and optionally also expresses T1R3 and/or TRPM5;
(xxv) the assay uses a GPR113-expressing cell which also recombinantly or endogenously expresses a G protein selected from Gi proteins, Gs proteins, Gq proteins, Ga15, Ga16, transducin or gustducin or a chimera thereof;
(xxvi) the assay uses a GPR113-expressing cell which expresses a G protein which comprises a chimera of a Gs and Gq;
(xxvii) the assay uses a GPR113-expressing cell which expresses a G protein which comprises a chimera of a Gs and Gq which consists of a Gs protein wherein at least the last 5-40 amino acids are substituted with those of Gq;
(xxviii) the assay detects the activity of said compound by GPR113 expressed by an endogenous cell or progeny thereof;
(xxix) the assay identifies compounds that elicit or modulate GPR113 associated taste;
(xxx) the assay is a functional assay that detects changes in signal transduction of constitutively active GPR113;
(xxxi) the assay detects changes in IP3 or IP3 metabolites including IP1;
(xxxii) the assay identifies compounds that elicit, mimic or modulate fat taste;
(xxiii) the assay identifies fat taste enhancers; or
(xxxiv) the assay detects compounds that modulate fat metabolism and/or which regulate fat consumption and dietary control.

8-40. (canceled)

41. A compound identified using the assay of claim 6.

42-43. (canceled)

44. A method of eliciting, mimicking, or modulating fat taste using a compound identified using an assay according to claim 6.

45. A food, beverage, cosmetic, therapeutic or nutraceutical containing a compound identified according to claim 6.

46-47. (canceled)

48. A functional assay according to claim 6 for identifying a compound having potential in vivo application for eliciting, mimicking, blocking, enhancing or modulating fat, lipid, or fatty acid associated taste (“fat taste”) comprising the following:
(i) contacting an isolated GPR113 receptor or a cell that expresses a nucleic acid encoding a human GPR113 receptor polypeptide or a fragment or chimera thereof that functionally responds to at least one of fat, lipid, or fatty acid compounds or an ortholog thereof or a nucleic acid encoding a polypeptide possessing at least 90% sequence identity to the polypeptide encoded thereby with at least one putative modulator compound;
(ii) detecting whether said compound elicits activation or modulates the activation of said GPR113 polypeptide by another ligand; and
(iii) identifying the compound as a potential taste or taste bud associated function modulator based on whether it elicits activation or modulates the activation of the GPR113 polypeptide by another ligand.

49. A functional assay according to claim 6 for identifying a compound having potential in vivo application for eliciting, mimicking, blocking, enhancing or modulating fat, lipid, or fatty acid associated taste (“fat taste”) comprising the following:
(i) contacting one or more cells that express a constitutively active GPR113 with a putative GPR113 modulatory compound,
(ii) detecting for any changes in signal transduction of said constitutively active GPR113 elicited by said compound; and
(iii) identifying the compound as a potential taste or taste bud associated function modulator based on whether it elicits activation or modulates GPR113 signal transduction.

50. The functional assay of claim 48, wherein:
(i) the cell further recombinantly or endogenously expresses a G protein and/or another protein selected from GPR40, GPR120, phospholipase-Cβ2, CD36, T1R3 and TRPM5;
(ii) the cell further recombinantly or endogenously expresses a G protein selected from Gi proteins, Gq proteins, Gs proteins, transducin, gustducin, Ga15, Ga16 or a chimera of any of the foregoing;
(iii) the cell further recombinantly or endogenously expresses a G protein which is a chimera of a Gs and Gq;
(iv) the cell further recombinantly or endogenously expresses a G protein chimera that consists of a Gs protein wherein at least the last 5-40 amino acids are substituted with those of Gq;
(v) it detects the effect of said compound on arrestin translocation;
(vi) it detects the effect of said compound on second messengers;
(vii) it detects the effect of said compound on second messengers including cAMP, cGMP or IP3 or a metabolite of IP3;
(viii) it detects changes in voltage or intracellular calcium;
(ix) it includes the use of a voltage-sensitive or calcium-sensitive dye;
it detects the effect of said compound on G protein activation by said receptor;
(x) the GPR113 sequence is linked to a reporter gene, optionally luciferase, alkaline phosphatase, or 3-galactosidase;
(xi) it screens a synthetic or natural compound library;
(xii) it uses a combinatorial compound library for screening;
the screened compounds are contained in a randomized library of small molecules;
(xiii) it is carried out by a high-throughput screening assay;
(xiv) it screens for compounds that enhance or inhibit the activation of the GPR113 receptor by a fat, lipid, fatty acid or a fat containing composition, e.g., wherein the fat, lipid or fatty acid or composition includes soybean, corn, coconut, peanut, olive, safflower, vegetable, fish and/or other animal derived oils, linoleic acid, oleic acid, and other non-trans and trans fatty acids;
(xv) it detects the effect of said compound on signal transduction,
(xvi) it detects changes in cellular polarization;
(xvii) it uses a voltage-clamp or patch-clamp technique;
(xviii) it is a GTPγ35S assay;
(xix) it is a fluorescent polarization or FRET assay;
(xx) it detects changes in adenylate cyclase activity;
(xxi) it detects changes in IP3 or IP3 metabolites such as IP1;
(xxii) it detects the effect of said compound on ligand-specific coupling of said receptor with a G protein;
(xxiii) it detects the effects of said compound on a neurotransmitter or hormone release;
(xxiv) the assay uses a cell wherein said GPR113 receptor is stably expressed;
(xxv) the assay uses a cell wherein said GPR113 receptor is transiently expressed;
(xxvi) the assay uses a cell wherein said GPR113 receptor is expressed under the control of an inducible promoter;
(xxvii) the assay uses an endogenous cell that expresses GPR113 optionally an endogenous cell present in foliate, circumvallate or fungiform papillae or is a gastrointestinal or neuronal cell or present in or derived from gastrointestinal epithelium;
(xxviii) the assay further includes testing the effect of said compound or a derivative thereof in a human or animal taste test;
(xxix) the assay uses a fluorescence plate reader (FLIPR);
(xxx) the assay uses a voltage imaging plate reader (VIPR) which is used to increase ion channel-dependent sodium or fluid absorption;
(xxxi) the assay uses a membrane potential dye selected from the group consisting of Molecular Devices Membrane Potential Kit (cat#8034), Di-4-ANEPPS (pyridinium, 4-(2-(6-(dibutylamino)-2-naphthalen-yl)ethenyl)-1-(3-sulfopropyl)-hydroxide, inner salt);
DiSBACC4(2)(bis-(1,2-dibarbituric acid)-trimethine oxanol); DiSBAC4(3) (bis-(1,3-dibarbituric acid)-trimethine oxanol); CC-2-DPME (Pacific Blue 1,2-dietradecanoyl-sn-glycerol-3-phosphoethanolamine, triethylammonium salt) and SBFI-AM (1,3-Benzenedicarboxylic acid, 4,4′-[1,4,10-trioxa-7,13-diazacyclopentadecane-7,13-diylbis(5-methoxy-6,1,2-benzofurandiyl)]bis-tetrakis[(acetyloxy)methyl]ester (Molecular Probes);
(xxxii) the identified compounds are evaluated in vivo for their effect on fat taste, fat metabolism, fat absorption, satiety, fat intake and serum triglyceride levels;
(xxxiii) the assay screens for compounds that specifically bind and/or modulate the activity of said taste specific polypeptide and based on said screening assay identifying compounds having potential therapeutic efficacy in treating or preventing a pathological condition involving fat metabolism, absorption or excretion; or
(xxxiv) the assay screens for compounds that specifically bind and/or modulate the activity of said taste specific polypeptide and based on said screening assay identifying compounds having potential to regulate fat, fatty acid or lipid dietary preference and/or modulate body weight, e.g., wherein the disease is selected from celiac disease, irritable bowel syndrome, inflammatory bowel disease, Crohn's disease, Sjögren's syndrome, gastritis, diverticulitis, or ulcerative colitis and other liver, gall bladder or gastrointestinal conditions or another metabolic disorder or the disorder is diabetes, obesity, a metabolic syndrome or fatty liver disease.

51-91. (canceled)

92. A transgenic rodent wherein the expression of GPR113 has been knocked out, optionally which has been further genetically engineered to express a human or non-human primate GPR113 gene.

93-94. (canceled)

95. A method of using a transgenic rodent according to any of claim 92 to screen the effects of the expression of GPR113 on fat taste or fat metabolism or serum triglycerides; or to screen for fat taste modulators or enhancers or which modulate fat metabolism.

96-99. (canceled)

100. The functional assay of claim 49, wherein:
(i) the cell further recombinantly or endogenously expresses a G protein and/or another protein selected from GPR40, GPR120, phospholipase-Cβ2, CD36, T1R3 and TRPM5;
(ii) the cell further recombinantly or endogenously expresses a G protein selected from Gi proteins, Gq proteins, Gs proteins, transducin, gustducin, Ga15, Ga16 or a chimera of any of the foregoing;
(iii) the cell further recombinantly or endogenously expresses a G protein which is a chimera of a Gs and Gq;
(iv) the cell further recombinantly or endogenously expresses a G protein chimera that consists of a Gs protein wherein at least the last 5-40 amino acids are substituted with those of Gq;
(v) it detects the effect of said compound on arrestin translocation;
(vi) it detects the effect of said compound on second messengers;
(vii) it detects the effect of said compound on second messengers including cAMP, cGMP or IP3 or a metabolite of IP3;
(viii) it detects changes in voltage or intracellular calcium;
(ix) it includes the use of a voltage-sensitive or calcium-sensitive dye;
it detects the effect of said compound on G protein activation by said receptor;
(x) the GPR113 sequence is linked to a reporter gene, optionally luciferase, alkaline phosphatase, or 3-galactosidase;
(xi) it screens a synthetic or natural compound library;
(xii) it uses a combinatorial compound library for screening;
the screened compounds are contained in a randomized library of small molecules;
(xiii) it is carried out by a high-throughput screening assay;
(xiv) it screens for compounds that enhance or inhibit the activation of the GPR113 receptor by a fat, lipid, fatty acid or a fat containing composition, e.g., wherein the fat, lipid or fatty acid or composition includes soybean, corn, coconut, peanut, olive, safflower, vegetable, fish and/or other animal derived oils, linoleic acid, oleic acid, and other non-trans and trans fatty acids;
(xv) it detects the effect of said compound on signal transduction;
(xvi) it detects changes in cellular polarization;
(xvii) it uses a voltage-clamp or patch-clamp technique;
(xviii) it is a GTPγ35S assay;
(xix) it is a fluorescent polarization or FRET assay;
(xx) it detects changes in adenylate cyclase activity;
(xxi) it detects changes in IP3 or IP3 metabolites such as IP1;
(xxii) it detects the effect of said compound on ligand-specific coupling of said receptor with a G protein;
(xxiii) it detects the effects of said compound on a neurotransmitter or hormone release;
(xxiv) the assay uses a cell wherein said GPR113 receptor is stably expressed;
(xxv) the assay uses a cell wherein said GPR113 receptor is transiently expressed;
(xxvi) the assay uses a cell wherein said GPR113 receptor is expressed under the control of an inducible promoter;
(xxvii) the assay uses an endogenous cell that expresses GPR113 optionally an endogenous cell present in foliate, circumvallate or fungiform papillae or is a gastrointestinal or neuronal cell or present in or derived from gastrointestinal epithelium;
(xxviii) the assay further includes testing the effect of said compound or a derivative thereof in a human or animal taste test;
(xxix) the assay uses a fluorescence plate reader (FLIPR);
(xxx) the assay uses a voltage imaging plate reader (VIPR) which is used to increase ion channel-dependent sodium or fluid absorption;
(xxxi) the assay uses a membrane potential dye selected from the group consisting of Molecular Devices Membrane Potential Kit (cat#8034), Di-4-ANEPPS (pyridinium, 4-(2-(6-(dibutylamino)-2-naphthalen-yl)ethenyl)-1-(3-sulfopropyl)-hydroxide, inner salt); DiSBACC4(2)(bis-(1,2-dibarbituric acid)-trimethine oxanol);
DiSBAC4(3) (bis-(1,3-dibarbituric acid)-trimethine oxanol); CC-2-DPME (Pacific Blue 1,2-dietradecanoyl-sn-glycerol-3-phosphoethanolamine, triethylammonium salt) and SBFI-AM (1,3-Benzenedicarboxylic acid, 4,4′-[1,4,10-trioxa-7,13-diazacyclopentadecane-7,13-diylbis(5-methoxy-6,1,2-benzofurandiyl)]bis-tetrakis[(acetyloxy)methyl]ester (Molecular Probes);
(xxxii) the identified compounds are evaluated in vivo for their effect on fat taste, fat metabolism, fat absorption, satiety, fat intake and serum triglyceride levels;
(xxxiii) the assay screens for compounds that specifically bind and/or modulate the activity of said taste specific polypeptide and based on said screening assay identifying compounds having potential therapeutic efficacy in treating or preventing a pathological condition involving fat metabolism, absorption or excretion; or
(xxxiv) the assay screens for compounds that specifically bind and/or modulate the activity of said taste specific polypeptide and based on said screening assay identifying compounds having potential to regulate fat, fatty acid or lipid dietary preference and/or modulate body weight, e.g., wherein the disease is selected from celiac disease, irritable bowel syndrome, inflammatory bowel disease, Crohn's disease, Sjögren's syndrome, gastritis, diverticulitis, or ulcerative colitis and other liver, gall bladder or gastrointestinal conditions or another metabolic disorder or the disorder is diabetes, obesity, a metabolic syndrome or fatty liver disease.
